(19) United States
(12) Patent Application Publication
(10) Pub. No.: US 2022/0356269 A1
(43) Pub. Date: Nov. 10, 2022

(54) METHOD FOR ISOLATING EXTRACELLULAR VESICLES

(71) Applicant: Imperial College Innovations Limited, London (GB)

(72) Inventor: Richard Kelwick, Jr., London (GB)

(21) Appl. No.: 17/762,931

(22) PCT Filed: Sep. 23, 2020
(86) PCT No.: PCT/GB2020/052302

§ 371 Date: Mar. 23, 2022

(30) Foreign Application Priority Data
Sep. 24, 2019 (GB) 1913716.5

(51) Int. Cl. C07K 17/08; C12N 1/20

(52) U.S. Cl. CPC C07K 17/08; C12N 1/205; C07K 2319/50; C07K 2319/70

(57) ABSTRACT

The present invention provides a gentle and low cost means to isolate extracellular vesicles, including exosomes, from a surrounding sample. In particular the invention relates to the use of a fusion protein and biopolymer beads in such methods. Methods, compositions and medical uses of such compositions are also provided.

### FIELD

The present invention relates to the field of extracellular vesicles such as exosomes, and specifically to the provision thereof.

### BACKGROUND

Extracellular vesicles (EVs) is a broad term that encapsulates a highly heterogenous mixture of cell-derived membranous vesicles, found in most bodily fluids (e.g. blood, saliva and urine), that have important functions in human health and disease (Raposo and Stahl, 2019; Théry et al., 2018). Many other cell types also produce EVs including prokaryotes and EVs can also be produced in vitro in mammalian tissue culture. EVs influence cellular behaviour and human physiology (e.g. immunoregulation and cellular homeostasis) through the intercellular transfer of proteins, lipids, metabolites and nucleic acids (e.g. miRNAs). EV compositions can indicate a complex molecular signature of the cells and cell states from which they originated. Indeed, tumour-derived-exosomes carry cancer/tumour cell hallmarks (Wang et al., 2017) (Bebelman et al., 2018). Thus, an array of liquid biopsies based around detecting EV-associated biomarkers are being developed (Kim et al., 2018; Roy et al., 2018).

Extracellular vesicles (EVs), including exosomes, are also a powerful class of nanovesicle therapeutics and drug delivery vehicles. The ability for EVs to deliver complex molecular cargoes and elicit changes in cellular behaviour is increasingly being exploited through the development of EV therapeutics and EV drug delivery systems (Armstrong et al., 2017; Lener et al., 2015; Ng et al., 2019; Watson et al., 2018). The exosome market is growing rapidly ($2.28 bn by 2030; CAGR 18.8%; GVR 2018). Indeed, an increasing number of early-phase clinical trials are demonstrating the efficacy of exosome therapeutics for the treatment of several different diseases—including cancers (Colao et al., 2018; Patel et al., 2018). Exosomes may also become the delivery vehicle of choice for gene-editing therapies (e.g. CRISPR-Cas; Kim et al., 2017) and oligonucleotide-therapeutics (e.g. ncRNAs) (Zhang et al., 2018). Several studies have shown that stem cell derived EVs can infer similar therapeutic modalities to that of the stem cells themselves (Willis et al., 2017). Furthermore, an array of clinical trials has also demonstrated promising results for EV therapeutics and EV drug delivery strategies against several disease indications for the treatment of cancers, neurodegenerative diseases and cardiovascular disorders (Zhang et al., 2019).

However, EV biology is highly complex and there are limitations with standard EV isolation methods that are hampering the scalable manufacture of therapeutic EVs. EV subtypes differ in their size, morphology, composition, molecular cargoes and biogenesis which has led to poorly defined EV nomenclature. Terms to describe EV subtypes include, but are not limited to, exosomes, microvesicles, small or large oncosomes, ectosomes, apoptotic bodies or exomeres. Generally, EVs such as exosomes are generated through secretion pathways that involve the fusion of multi-vesicular endosomal bodies (MVB) with the plasma membrane (Raposo and Stahl, 2019). Whereas, microvesicles are simply shed from the plasma membrane. Arguably, exosomes have so far received the most attention for biomarker and therapeutic applications. However, their specific isolation, away from other EV subtypes, and other cellular components (e.g. secreted proteins, cell-free nucleic acids) is incredibly challenging (Ng et al., 2019).

The most commonly used exosome isolation method is ultracentrifugation which uses centrifugal forces (>100,000×g) to separate exosomes from cells. Yet, ultracentrifugation causes exosome aggregation and each batch varies in terms of the cell debris, other EV subtypes and protein aggregates that are co-isolated along with the exosomes. Gentler exosome isolation methods such as tangential flow filtration and size exclusion chromatography may be scaled-up. However, they also often result in the isolation of mixed exosome populations, along with cellular contaminants. Standard antibody-beads directed against exosome-surface markers (e.g. TSG101, CD9, CD63, CD82) may enable specific exosomes to be captured. However, once captured the methods used to de-couple exosomes from the antibody beads (SDS or low pH) actually damages the exosomes—potentially hindering their therapeutic activity (Konoshenko et al., 2018) (Cheng et al., 2019). A recent study also determined that none of these exosome isolation methods were economically scalable for generating clinical-grade exosomes (Ng et al., 2019).

Therefore, improved exosome isolation methods are urgently needed to address this bottleneck to ensure continued market growth and for future exosome manufacturing to meet the needs of large patient groups (Ng et al., 2019).

The present invention solves this problem by providing a scalable, low cost method of capturing and releasing EVs in a gentle manner that doesn't negatively affect the captured EVs. A further advantage of the present invention is that it allows an engineered peptide or protein (e.g. sfGFP, His-tag or cell targeting peptide) to be displayed on the surface of the released EVs/exosomes. Such an engineered peptide or protein could be a cell targeting peptide(s) or cell membrane disrupting peptide(s) that can enhance the delivery of the EVs/exosomes to specific tissues or organs (Wang et al., 2017; Wiklander et al., 2015) (U et al., 2018).

### SUMMARY OF THE INVENTION

The inventors have developed a scalable and modular platform for the isolation of extracellular vesicles (including exosomes) that is based upon functionalised, microbially generated, biopolymer particles, such as polyhydroxyalkanoate (PHA) biopolymer particles. These biopolymer particles incorporate on their surface, novel, engineered, fusion proteins that include PHA binding domains/proteins, and in some embodiments include EV binding peptides/affibodies and a proteolytic cleavage site that can be used to gently release captured exosomes.

The biopolymer particle/fusion protein arrangement can also be used in the field of medical diagnostics due to their ability to capture and purify EVs, such as exosomes or oncosomes, that comprise a particular antigen.

The biopolymer particle/fusion protein arrangement can also be used, via cleavage of the proteolytic cleavage site to generate engineered EVs which comprise a surface-bound polypeptide, which may be associated with a particular function.

### DETAILED DESCRIPTION OF THE INVENTION

The invention is as defined by the claims.

In one aspect, the invention provides a method of producing biopolymer particles coated with a fusion protein, wherein the method comprises the steps of:
(i) providing a host cell that produces
   a) biopolymer particles; and
   b) a fusion protein capable of coating the biopolymer particles in the cells, wherein the fusion protein comprises a biopolymer particle binding domain and an extracellular vesicle binding domain and a sequence capable of being cleaved by a protease, optionally a site specific protease, optionally a TEV protease;
(ii) cultivating the host cell under conditions suitable for the production of biopolymer particles coated with the fusion protein.

In further embodiments, the method further comprises isolating the coated biopolymer particles from the host cell.

The coated biopolymer particles produced by the invention may be used in any of the methods described herein and are useful in methods that require the isolation of extracellular vesicles for example for diagnostic reasons, or for the production of therapeutic extracellular vesicles. Assembling the coated biopolymer particle within a cell is considered to be advantageous over other methods which require the initial separate production of the biopolymer particle and the fusion protein, isolation and purification of the biopolymer particle and the fusion protein, and subsequent contacting between the biopolymer particle and the fusion protein, followed by any necessary further clean up/purification steps. The present “one-pot” method allows the simultaneous production of both the fusion protein and biopolymer particle in the same cell, along with concomitant contacting between the two inside the cell. Accordingly, only a single isolation and purification step is required.

In preferred embodiments, the expression of the genes required to make the biopolymer particle, and the gene that encodes the fusion protein, are under the expression of different promoters, for example different inducible and/or repressible promoters. In this way, expression of the fusion protein and biopolymer particle can be separately finely tuned so that appropriate expression levels of each is achieved. For example, in one embodiment, the cell may comprise a vector that comprises a phaCAB operon and a vector that comprises a gene encoding the fusion protein. In particular embodiments, the genes encoding the biopolymer particle and the fusion protein are part of the same nucleic acid molecule, for example are part of the same nucleic acid vector. Accordingly, in one embodiment, expression of the biopolymer particle is under the control of a different promoter to the expression of the fusion protein, optionally wherein the biopolymer particle is under the control of a first inducible promoter and the fusion protein is under the control of a second inducible promoter.

The skilled person will understand what is meant by an inducible promoter, and is able to select appropriate inducible promoters.

It will be appreciated however that many of the methods of using the coated biopolymer particles can be put into effect using coated biopolymer particles that have been made by any method.

Preferences for features of this aspect of the invention, e.g. biopolymer particle material, host cell, isolation methods, biopolymer particle size, % coating of the biopolymer particle, number of biopolymer particles in the host cell, fusion protein including biopolymer particle binding domains and extracellular vesicle binding domain and functionalisation domain, linkers, sequences capable of being cleaved by a protease, number of different fusion proteins coating the biopolymer particle, for example, are as defined below and throughout.

In another aspect, the invention provides a method for isolating extracellular vesicles from a sample, the method comprising:
(i) providing a composition of coated biopolymer particles, the biopolymer particles being coated with a fusion protein;
(ii) contacting the composition comprising the coated biopolymer particles with the sample comprising extracellular vesicles under conditions which allow the formation of a coated biopolymer particle-extracellular vesicle complex; and
(iii) isolating the coated biopolymer particle-extracellular vesicle complex.

The method of producing coated biopolymer particles and the method for isolating extracellular vesicles (EV) utilises a fusion protein which is attached to the biopolymer particle, which in some instances can be considered to be a bioplastic bead, and which fusion protein can also bind to and capture EVs, or to for example a protein that is located in the membrane of the EV. Since the biopolymer particles are relatively large and in some instances are “bead-like”, they are simple to isolate from the surrounding matrix, for example surrounding cell lysate or solution, by, for example, filtration. “bead-like” biopolymer particles are also easy to analyse, for example by flow cytometry and are compatible with automation, for example by liquid handling robots.

By EV we include the meaning of any lipid bound vesicle that is outside of a cell. For example, we include the meaning of synthetically produced vesicles of which the skilled person will be aware. The present isolation methods are also considered to be useful in the isolation of synthetically produced vesicles, or liposomes. For example, in some instances the synthetic vesicles or liposomes may be functionalised in vitro, and the methods of the present invention can be used to isolate the functionalised vesicles/liposomes from the cell-free extract, energy mix and other components. Accordingly, in some instances, the term extracellular vesicle encompasses vesicles that have not strictly been produced by a cell. Accordingly, in one embodiment the term extracellular vesicle includes the meaning of a lipid bound vesicle.

In other embodiments, the term EV is intended to mean a lipid bound vesicle that was produced by a cell and released into the surrounding medium, for example in some embodiments the term EV is intended to mean a lipid bound vesicle generated through secretion pathways that involve the fusion of multi-vesicular endosomal bodies (MVB) with the plasma membrane (Raposo and Stahl, 2019). In some embodiments, the term EV includes microvesicles that are shed from the plasma membrane. In other embodiments the term EV does not include microvesicles.

In a preferred embodiment, the EVs are vesicles produced by a cell and released into the surrounding medium. In some embodiments the EV is a microvesicle, an apoptotic body, an ectosome, an exosome, an exomere, a small oncosomes, or a large oncosome, that has been produced by a cell. In some embodiments the EV is an exosome mimetic, or an exosome mimetic nanovesicle. The skilled person will understand what is meant by an exosome mimetic and includes, for example, a cell that has been extruded down to the size of an EV or to an exosome (see for example Jun-Yi W et al 2018 Scientific Reports 8: article number 2471).

In particular embodiments the EV is an exosome.

The EV may be associated with a particular antigen, such as a protein or proteins that can be located in the lipid membrane. Preferably the EV is associated with at least one antigen, such as a protein or nucleic acid, or for example is a microvesicle, an apoptotic body, an ectosome, an exosome, an exomere, a small oncosomes, or a large oncosome that is associated with at least one antigen such as a protein, preferably wherein the antigen is located in the membrane of the EV.

The sample comprising the EVs may be any type of sample. In some embodiments the sample is a cell lysate or a media taken from a cell culture.

In some embodiments the EVs comprise a factor to which the fusion protein can bind to. The factor to which the fusion protein binds could be a lipid, surface protein or other molecule that might or might not induce an immune response. For example, the factor could be located in the EV membrane or on the surface of the EV membrane or otherwise bound strongly to the surface of the EV. Suitable factors include, but are not limited to: EV membrane lipids; Proteins involved in endocytosis/multivesicular body and/or EV biogenesis, including TSG101, ALIX, and Lamp2b; Proteins involved in miRNA processing e.g. Argonaut 2 and Y-box protein-1; Tetraspanins including: CD63, CD9, CD81; Proteins involved in miRNA processing e.g. Argonaut 2 and Y-box protein-1; Cell surface receptors including: HER2/neu, EGFR, Syndecans 1, 2, 3 and 4; Integrins: all alpha and beta combinations such as α1β1, α2β1, α3β1, α4β1, α5β1, α6β1, α7β1, α8β1, α9β1, α10β1, α11β1, αVβ1, αLβ2, αMβ2, αXβ2, αDβ2, αIIbβ3, αVβ3, α6β4, αVβ5, αVβ6, α4β7, αEβ7, αVβ8; Metalloproteinases including all MMPs, ADAMs and ADAMTSs; Major Histocompatibility Complex (MHC) proteins; Engineered fusion proteins expressed to integrate within exosome membranes e.g. CD63, CD81 or Lamp2b N or C terminal fusions.

In some embodiments the coated biopolymer particles and the method is for isolating specific EVs from a sample that may comprise other EVs that are not to be isolated. The specificity of the isolation of the target EVs is derived from the ability of the fusion protein to recognise and bind to an antigen located in the membrane of, or bound to, the target EV.

The invention requires a biopolymer particle that is coated with a fusion protein. The biopolymer particle itself may be any biopolymer particle. For example, the biopolymer particle may be a protein or polypeptide particle, a nucleic acid particle, a carbohydrate particle. Preferably the biopolymer particle is a biological molecule that can be produced by a cell, such as a bacterial cell, optionally a cyanobacterial cell; an archaeal cell, optionally a haloarchaeal cell; a fungal cell, optionally a yeast cell; or a plant cell.

Preferably the coated biopolymer particle is produced in a cell according to the method of the invention.

However, the skilled person will appreciate that biopolymers, such as those described herein, do not have to be made biologically within a cell. In some embodiments of the method of isolating extracellular vesicles therefore the biopolymer particle is not made within a cell and is instead made synthetically. In these instances, the biopolymer particle may be contacted with the fusion protein in vitro to result in the coated biopolymer particle. Likewise, the biopolymer particle produced by a cell may be isolated from the cell and contacted with the fusion protein in vitro. It will also be appreciated that, particularly in instances where the biopolymer particle is not made within the same cell as the fusion protein, the biopolymer particle does not have to be a “particle” per se, and could for example, take the form of a film. The film can then be contacted with the fusion protein in vitro to form, for example, a fusion protein coated array. However, there are advantages provided by the present invention in producing the biopolymer particle and the fusion protein in the same cell, as described herein. For instance, in one embodiment the fusion protein does not require separate purification, since it will interact with the biopolymer particle in vivo, i.e. inside the cell in which both the biopolymer particle and the fusion protein are produced, allowing the fusion protein to coat the biopolymer particle and the fusion protein and biopolymer particle to be isolated together. In one embodiment then the fusion protein and the biopolymer particle are produced in the same cell.

In preferred embodiments of producing coated biopolymer particles and the method of isolating extracellular vesicles, the biopolymer particle comprises a polymer that is able to bind to any one or more of
a) PhaR-derived binding domain (PBD), optionally comprises or consists of SEQ ID NO: 2 or SEQ ID NO 1 or a sequence that has at least 70%, 75%, 80%, 85%, 90%, 91%, 92%, 93%, 94%, 95%, 96%, 97%, 98%, 99%, or 100% sequence identity to SEQ ID NO: 1 or SEQ ID NO: 2
b) a phasin, optionally a PhaR, a PhaP, a PhaQ, a PhaF, a PhaI, or an inactive PhaZ1;
c) IbpA (HspA);
d) PhaC.

The skilled person is aware of the binding properties of the above proteins, and is aware of the polymers to which they are able to bind.

In some embodiments, the biopolymer particle is selected from the group consisting of a polymer that comprises polyhydroxyalkanoate (PHA), a poly(L-lactide) (PLLA), a polythioester (PTE), a polyethylene (PE), or a polystyrene (PS).

In some embodiments the biopolymer particle is considered to be a bioplastic polymer. However, although PS and PE are not considered to be bioplastics they are also considered to be suitable for use with the present invention. In some embodiments therefore the biopolymer is a bioplastic polymer or is PS or PE, or a combination of one or more of these.

For example, the biopolymer particle may be a class of particle that comprises PHA, for example that comprises one or more of poly(3-hydroxybutyrate) (P(3HB)), poly(4-hydroxybutyrate) (P(4HB)), polyhydroxyvalerate (PHV), poly(3-hydroxyhexanoate) (P(3HHx)), poly(3-hydroxyheptanoate) (P(3HH)), poly(3-hydroxyoctanoate) (P(3HO)), poly(3-hydroxynonanoate) (P(3HN)), poly(3-hydroxydecanoate) (P(3HD)), poly(3-hydroxybutyrate-co-3-hydroxyvalerate) (PHBV), 3-hydroxybutyrate and 4-hydroxybutyrate (P3HB4HB), poly(3HB-co-3-hydroxyvalerate) (P(3HB-co-3HV)), poly(3HB-co-3-hydroxyhexanoate) (P(3HB-co-3HHx)), poly(3HB-co-3-hydroxy-4-methylvalerate) (P(3HB-co-3H4MV)), or poly(3HB-co-medium-chain-length-3HA) (P(3HB-co-mcl-3HA)).

In some embodiments the biopolymer particle comprises PLLA, PE, PS or PTE.

In some embodiments the biopolymer particle comprises any one of more of PHA, PLLA, PE, PS or PTE.

In some instances, the biopolymer particle is a blended polymer. By a blended polymer we include the meaning that the particle is made from 2 or more different subunits or monomers, for example is made from at least 2 monomers selected from the group consisting of PHA, poly(lactic acid)-poly(hydroxybutyrate) (PLA-PHB), starch-PHA, poly(L-lactide) (PLLA), a polyethylene (PE), a polystyrene (PS), or a polythioester (PTE). In a preferred embodiment, the blended polymer comprises PHA. The blended polymer may comprise at least 2, 3, 4, 5, 6, 7, 8, 9, or at least 10 different monomers.

In a particular embodiment, the PHA-blended biopolymer comprises poly(lactic acid)-poly(hydroxybutyrate) (PLA-PHB) or starch-PHA.

In some instances, the biopolymer is not a blended biopolymer.

By bioplastic we include the meaning that the bioplastic is made biologically, for example within a cell as described herein. However, as described above, the skilled person will understand that the same “bioplastic” can be made in a non-biological way. Such non-biologically produced bioplastics are also considered to be useful in methods of using the coated biopolymer particles, and in some embodiments of these methods the biopolymer particle is a particle made from a non-biologically produced bioplastic.

Preferably, the biopolymer particle or bioplastic particle is made according to the method of the invention and is made biologically within a cell as described herein. As discussed herein, there are benefits associated with producing the biopolymer particle and the fusion protein within the same cell. The skilled person will understand which appropriate biopolymers can be made within a cell, and is able to determine the necessary genes and constructs required to produce said biopolymer particle within a given cell.

The biopolymer particle may be of any shape and any size. Different shapes are considered to have different applications. For example, biopolymer particles that have been extruded into fibres are considered to have a different application to spherical bead-like biopolymer particles. Typically, the biopolymer particle will be spherical, or substantially spherical. In this embodiment, the particle can be considered to be a bead or bead-like. For example, the biopolymer particle may be considered to be a bioplastic bead. Advantages of bead-like biopolymer particles are discussed above, such as their ease of analysis by methods such as flow cytometry and suitability for use in automated methods. Spherical, or substantially spherical biopolymer particles are also considered to have an advantageous surface area for binding to the EV. The skilled person will be aware of techniques to determine the size and shape, for example bead size can be analysed using dynamic light scattering or flow cytometry, for example using a Malvern Zetasizer Nano ZS (Malvern Instruments, Malvern, UK) DLS system.

In some embodiments the particle is the shape of a cell, for example where the particle is a biopolymer particle, such as a bioplastic particle, that has been made in a cell, the particle will tend to be bounded by the shape and size of the cell. In some embodiments the particle is in the form of an array, for example may be square or rectangular. In some embodiments the particle is not in the form of an array, for example is not square or rectangular.

In some embodiments the mean diameter of the uncoated biopolymer particle is between 50 nm and 1,500 nm, for example between 60 nm and 1,250 nm, 80 nm and 1,000 nm, 100 nm and 800 nm, 150 nm and 600 nm, 200 nm and 500 nm, 300 and 400 nm. In the same or different embodiments, the mean diameter is less than 1,500 nm, 1,250 nm, 1,000 nm, 800 nm, 600 nm, 500 nm, 400 nm, 300 nm, 200 nm, 150 nm, 100 nm, 80 nm, 60 nm, or less than 50 nm. In the same or other embodiments the mean diameter is greater than 1,500 nm, 1,250 nm, 1,000 nm, 800 nm, 600 nm, 500 nm, 400 nm, 300 nm, 200 nm, 150 nm, 100 nm, 80 nm, 60 nm, or greater than 50 nm.

In a particular embodiment, the mean diameter of the uncoated biopolymer particle is between 500 nm and 1,500 nm.

In the same or different embodiments, the mean diameter of the uncoated biopolymer particle is at least 300 nm.

The biopolymer particles required by the invention are coated with a fusion protein. The fusion protein has the ability to both bind to the biopolymer particle (or to a biopolymer film in particular embodiments), and also to bind to a factor or antigen on the surface of the EV of embedded within the EV membrane. The factor to which the fusion protein binds could be a lipid, surface protein or other molecule that might or might not induce an immune response. Examples of appropriate factors are discussed above and include EV membrane lipids; Proteins involved in endocytosis/multivesicular body and/or EV biogenesis, including TSG101, ALIX, and Lamp2b; Proteins involved in miRNA processing e.g. Argonaut 2 and Y-box protein-1; Tetraspanins including: CD63, CD9, CD81; Proteins involved in miRNA processing e.g. Argonaut 2 and Y-box protein-1; Cell surface receptors including: HER2/neu, EGFR, Syndecans 1, 2, 3 and 4; Integrins: all alpha and beta combinations such as α1β1, α2β1, α3β1, α4β1, α5β1, α6β1, α7β1, α8β1, α9β1, α10β1, α11β1, αVβ1, αLβ2, αMβ2, αXβ2, αDβ2, αIIbβ3, αVβ3, α6β4, αVβ5, αVβ6, α4β7, αEβ7, αVβ8; Metalloproteinases including all MMPs, ADAMs and ADAMTSs; Major Histocompatibility Complex (MHC) proteins; Engineered fusion proteins expressed to integrate within exosome membranes e.g. CD63, CD81 or Lamp2b N or C terminal fusions The concept of fusion proteins is well known in the art and typically a fusion protein comprises at least 2 regions taken from 2 different proteins that are transcribed and translated into a single protein. For example, a protein tagged with GFP is a fusion protein. The at least 2 regions may comprise mutations with respect to the original protein from which the regions were taken. The regions may represent fragments or domains of a larger parent protein and so may comprise only some of the functions of the parent protein(s), or the regions may represent the entire parent protein itself and corresponding functions. The term fusion protein also includes the meaning of combining at least two functional domains, wherein the functional domains may be taken from the same or from different proteins.

In some embodiments, the invention provides or uses at least two different fusion protein coated biopolymer particles. For example in one embodiment of producing the coated biopolymer particles, the host cells expresses at least two different fusion proteins, and/or the host cell produces at least two different types pf biopolymer particle. The method of isolating extracellular vesicles may likewise utilise at least two different fusion protein coated biopolymer particles.

In the same or different embodiment, the method provides or uses at least two different fusion protein coated biopolymer particles that comprise different fusion proteins.

The biopolymer particles of the invention may be coated with one or more different fusion proteins. In this way, different functions or properties can be imparted to the biopolymer particle. For example, the ability to bind to and isolate more than one different type of EV such as more than one type of exosome. The more than one different fusion proteins may have the same biopolymer particle binding domain, but each have a different EV binding domain. Alternatively, in some embodiments the more than one different fusion protein may have the same EV binding domain but different biopolymer particle binding domains.

In a further embodiment, the more than one fusion protein may have different EV binding domains and different biopolymer particle binding domains. The biopolymer particle may be coated with at least 1, 2, 3, 4, 5, 6, 7, 8, 9 or at least 10 different fusion proteins as described herein, and in some embodiments can bind to at least 1, 2, 3, 4, 5, 6, 7, 8, 9 or or more different factors that are located on the EV membrane or in the EV membrane.

In similar embodiments, where the biopolymer particle is coated with more than one different fusion protein, directed towards different factors on or in the EV membrane, the coated particle could be used in a similar way to a bispecific antibody, i.e. each of the different EV binding domains though targeted to a different factor, can be targeted to different factors on the same EV. Such a method is considered to have particular utility in the isolation of low abundant EVs, or EVs that have a low abundance of particular target factors.

In one embodiment the fusion protein of the invention has one domain that has the ability to bind specifically to the biopolymer particle such as a bioplastic polymer particle (the biopolymer particle binding domain) and has at least another domain that binds specifically to the EV that is to be isolated (the EV binding domain). By “bind specifically to the biopolymer particle” we include the meaning that the fusion protein is able to bind to the particular bioplastic polymer from which the particle is formed, and is not able to bind to any other, or is not able to substantially bind to any other biopolymer. However, in some embodiments the domain that has the ability to bind specifically to the biopolymer particle is able to bind specifically to a class of biopolymer particles, for example is able to bind to biopolymer particles that comprise PHA, which as described above represents a class of biopolymers.

Preferably, the biopolymer particle binding domain binds only to the target biopolymer particle and the EV binding domain binds only to the target EV. Such binding specificity may be determined by methods well known in the art, such as ELISA, immunohistochemistry, immunoprecipitation, Western blots and flow cytometry.

Advantageously, in some embodiments the biopolymer particle binding domain is capable of binding selectively to the target biopolymer particle and the EV binding domain binds is capable of binding selectively only to the target EV, i.e. it binds at least 10-fold more strongly to the target biopolymer particle and to the EV than to any other biopolymer particle, factor or antigen.

In some embodiments the biopolymer particle binding domain binds to the target biopolymer particle with a higher Kd than to other factors, antigens or biopolymer particles; and/or the EV binding domain binds to the target EV with a higher Kd than to other factors, antigens or biopolymer particles. Therefore, typically, the Kd for the biopolymer particle binding domain to the target biopolymer particle, and the Kd for the EV binding domain to the target factor of antigen on the target EV will be 2-fold, preferably 5-fold, more preferably 10-fold less than Kd with respect to the other, non-target molecules such as non-target biopolymer particles, factors or antigens. More preferably, the Kd will be 50-fold less, even more preferably 100-fold less, and yet more preferably 200-fold less.

Methods for measuring the overall affinity (KD) and on-rate (ka) and off-rate (kd) of an interaction (such as an interaction between an antibody and a ligand) are well known in the art. Exemplary in vitro methods are described in the accompanying Examples. It is also conceivable to use flow cytometry based methods (Sklar et al., Annu Rev Biophys Biomol Struct, (31), 97-119, 2002).

Where the coated biopolymer particle has been produced in a cell in accordance with the first aspect of the invention, the skilled person will appreciate that the fusion protein is designed so that the biopolymer particle binding domain is able to bind to the specific biopolymer particle that is being produced in the cell. In this instance, provided that the cell is not also producing a different biopolymer particle, it is not essential that the biopolymer particle binding domain is not able to bind to other biopolymer particles. Where the coated biopolymer particle is produced in a cell, the skilled person will understand that the biopolymer particle binding domain should not be able to bind to, or should not be able to substantially be able to bind to, other cellular components present in the cell in which the particle is made.

The biopolymer particle binding domain can be any protein domain that is able to bind to the biopolymer particle. For example, where the biopolymer is a proteinaceous biopolymer or a carbohydrate biopolymer or a bioplastic polymer, the biopolymer binding domain can be an antibody, or a region of an antibody that can recognise the particular biopolymer particle.

For example, the biopolymer binding domain can be a protein fragment, a binding domain, a target-binding domain, a binding protein, a binding protein fragment, an affibody, an antibody, an antibody fragment, an antibody heavy chain, an antibody light chain, a single chain antibody, a single-domain antibody, a Fab antibody fragment, an Fc antibody fragment, an Fv antibody fragment, a F(ab′)2 antibody fragment, a Fab′ antibody fragment, a single-chain Fv (scFv) antibody fragment, a camelid antibody, an IgNAR Shark antibody, a DARPin, a nanobody, an antibody binding domain, biotin, a biotin derivative, an avidin, a streptavidin, a substrate, an enzyme, an abzyme, a co-factor, a receptor, a receptor fragment, a receptor subunit, a receptor subunit fragment, or a ligand.

In one embodiment the biopolymer binding domain is not a camelid antibody or a camelid-derived antibody.

In particular embodiments, where the biopolymer particle is a bioplastic particle such as a bioplastic bead, the biopolymer particle binding domain can be considered to be a bioplastic bead binding domain and can, be for example, a domain that can bind to a polyhydroxyalkanoate (PHA), a poly(L-lactide) (PLLA), a polyethylene (PE), a polystyrene (PS), or a polythioester (PTE); for example can bind to a particle that comprises PHA, for example that comprises one or more of poly(3-hydroxybutyrate) (P(3HB)), poly(4-hydroxybutyrate) (P(4HB)), polyhydroxyvalerate (PHV), poly(3-hydroxyhexanoate) (P(3HHx)), poly(3-hydroxyheptanoate) (P(3HH)), poly(3-hydroxyoctanoate) (P(3HO)), poly(3-hydroxynonanoate) (P(3HN)), poly(3-hydroxydecanoate) (P(3HD)), poly(3-hydroxybutyrate-co-3-hydroxyvalerate) (PHBV), 3-hydroxybutyrate and 4-hydroxybutyrate (P3HB4HB), poly(3HB-co-3-hydroxyvalerate) (P(3HB-co-3HV)), poly(3HB-co-3-hydroxyhexanoate) (P(3HB-co-3HHx)), poly(3HB-co-3-hydroxy-4-methylvalerate) (P(3HB-co-3H4MV)), or poly(3HB-co-medium-chain-length-3HA) (P(3HB-co-mcl-3HA)).

In the same or other embodiments the biopolymer particle binding domain, or bioplastic bead binding domain can bind to a polymer that comprises monomers of PHA, poly(lactic acid)-poly(hydroxybutyrate) (PLA-PHB), starch-PHA, poly(L-lactide) (PLLA), a polyethylene (PE), a polystyrene (PS), or a polythioester (PTE); or can bind to a blended polymer that comprises more than 2 different monomers, for example more than 2 monomers selected from the group consisting of PHA, poly(lactic acid)-poly(hydroxybutyrate) (PLA-PHB), starch-PHA, poly(L-lactide) (PLLA), a polyethylene (PE), a polystyrene (PS), or a polythioester (PTE).

In a particular embodiment, the biopolymer particle binding domain can bind to a PHA-blended biopolymer comprising poly(lactic acid)-poly(hydroxybutyrate) (PLA-PHB) or starch-PHA.

The skilled person is aware of suitable bioplastic binding domains. Examples of suitable domains include
a) PhaR-derived binding domain (PBD)
b) a phasin, optionally a PhaR, a PhaP, a PhaQ, a PhaF, a PhaI, or an inactive PhaZ1;
c) IbpA (HspA); or
d) PhaC.

The present inventors have generated a PhaR-derived binding domain (PBD) [SEQ ID NO: 2] which is considered to be advantageously small in size.

See for example Maestro and Sanze 2017 Microb Biotechnol 1323-1327; Neumann et al 2008 J Bacteriol 190: 2911-2919; Lee et al 2004 J Bacteriol 186: 3015-3021; Cai et al 2009 Bioresour Technol 100: 2265-2270; Brigham et al 2012 AMB Express 2:26; Tessmer et al 2007 Microbiology 153: 366-374; Wang et al 2011 10:21; Hay et al 2015 Microbial Cell Factories 14: 190.

Since one function of the fusion protein is to act as a specific bridge between the biopolymer particle and the target EVs, it is preferred if the biopolymer particle binding domain remains bound to the particle, for example remains bound to the particle during processes such as isolation of the coated particle from a cell, in instances where the coated particle is formed in a cell, and for example remains bound to the particle during complex formation between the coated biopolymer particle and the EV, and for example remains bound to the particle during separation or isolation of the coated biopolymer particle-EV complex. Such suitable biopolymer particle binding domains include the antibody like domains described herein. Other suitable domains include a) a phasin, optionally a PhaP, a PhaR, a PhaQ, a PhaF, a PhaI, or an inactive PhaZ1; b) IbpA (HspA); c) PhaR-derived binding domain (PBD); or d) PhaC.

Accordingly, in some embodiments, the fusion protein comprises a biopolymer binding domain that is optionally a PhaP, a PhaR, a PhaQ, a PhaF, a PhaI, or an inactive PhaZ1 domain; b) IbpA (HspA) domain; c) PhaR-derived binding domain (PBD) domain; or d) PhaC domain.

In some specific embodiments, the biopolymer binding domain is not or does not comprise PhaC, PhaZ and/or PhaP.

In some embodiments, the biopolymer binding domain of the fusion protein is a domain that is involved in the production of the biopolymer itself. For example PhaA, PhaB and PhaC are responsible for producing the PHA polymers (Kelwick et al 2018 Synthetic Biology 3(1): ysy016; Kelwick et al 2015 PLOS ONE 20: DOI:10.1371/journal.pone.0117202); the Phasins regulate gene expression and/or bind to polymer bead affecting the size, shape/granule density etc. of the beads (Maestro and Sanze 2017 Microb Biotechnol 1323-1327; Mariela et al DOI: 10.1128/AEM.01161-16); and PhaZ1 degrades the polymer so that the bacteria can utilise it as a carbon source

In some preferred embodiments, although domains such as PhaC are considered to be suitable for use in the present invention, and their incorporation into the fusion protein is considered useful, in some instances it is preferred if the biopolymer particle binding domain has a smaller size than that of PhaC. Due to their size, large binding domains preclude the binding of larger numbers of fusion proteins to a single biopolymer particle. By using smaller binding domains, a greater number of fusion proteins can bind to the particle, resulting in a higher % coating of the particle with the fusion protein. This results in a higher number of EV binding domains per particle available to bind to the target EV. PhaC for example has a size of around 64.38 kDa, whereas IbpA has a size of around 16 kDa and PhaR (PBD) has a size of around 10.69 kDa. The skilled person will be able to determine the size of a particular biopolymer particle binding domain, and is able to select the most appropriate sized domain to incorporate into the fusion protein depending on, for example, the size of the biopolymer particle, and the degree of coating with the fusion protein that is required. Accordingly, in some embodiments, the biopolymer binding domain is smaller in size than the PhaC domain, for example is smaller than 64 kDa in size.

In some embodiments the biopolymer binding domain is an engineered domain from PhaR, for example which has been engineered to reduce the size of the biopolymer binding domain. In some embodiments, the engineered PhaR domain consists of or comprises any one or more of the following sequences, or a sequence that has at least 70%, 75%, 80%, 85%, 90%, 91%, 92%, 93%, 94%, 95%, 96%, 97%, 98%, 99%, or 100% sequence identity to any one or more of the following sequences:

Full length wildtype PhaR
[SEQ ID NO: 1]
>WP_010810130.1 MULTISPECIES: polyhydroxyalkanoate
synthesis repressor PhaR
[Cupriavidus]
MATTKKGAERLIKKYPNRRLYDTQTSTYITLADVKQLVMDSEEFKVVDA
KSGDELTRSILLQIILEEETGGVPMFSSAMLSQIIRFYGHAMQGMMGTY
LEKNIQAFIDIQNKLAENSKGLYSGETFSPDMWSQFMNMQGPMMQGMMS
NYIEQSKNLFVQMQEQMQSQAKNMFGTFPFNQPDKK

Pfam DNA Binding Region

PhaR-derived binding domain (PBD)
[SEQ ID NO: 2]
MFSSAMLSQIIRFYGHAMQGMMGTYLEKNIQAFIDIQNKLAENSKGLYS
GETFSPDMWSQFMNMQGPMMQGMMSNYIEQSKNLFVQMQEQMQ

In preferred embodiments, the engineered PhaR domain consists of or comprises SEQ ID NO: 2, or consists of or comprises a sequence that has at least 70%, 75%, 80%, 85%, 90%, 91%, 92%, 93%, 94%, 95%, 96%, 97%, 98%, 99%, or 100% sequence identity to SEQ ID NO: 2.

In some embodiments,

a) between about 5% and 60% of the surface of the biopolymer particle is coated with the fusion protein, for example between 10% and 50%, 20% and 40%, for example 20% or 30% of the surface is coated with the fusion protein;
b) at least 5%, 10%, 20%, 30%, 40%, 50% or at least 60% of the surface is coated with the fusion protein; and/or
c) less than 60%, 50%, 40%, 30%, 20%, 10% or less than 5% of the surface is coated with the fusion protein.

The term “coated” takes its usual meaning. For example, by coated we mean that the biopolymer particle is bound on its external surface by the fusion protein. The biopolymer particle is considered to be coated by the fusion protein if the biopolymer particle is bound by one or more molecules of the fusion protein. For example, in some embodiments, the term coated is taken to mean that the biopolymer particle is bound by at least 1, 2, 3, 4, 5, 6, 7, 8, 9, 10, 20, 50, 100 molecules of the fusion protein. Typically, around 1,400-2,000 PhaC polypeptides are able to bind to the biopolymer particle of the invention; and up to around 40,000 fusion proteins are able to bind to each biopolymer particle.

The skilled person is able to calculate the expected number of molecules able to coat a particular biopolymer particle. For example, based on 1000 nm bead with 3.14×106 nm³ surface area, and a typical minimal radius of 93 kDa protein of 2.84 Rmin (nm), it is anticipated that hundreds to several thousands of Phasins would be located on the particle surface when these are expressed strongly in recombinant E. coli

In some embodiments, the biopolymer particle is considered to be coated with the fusion protein is at least 10% of the surface area of the particle is coated with the fusion protein, for example at least 15%, 20%, 25%, 30%, 35%, 40%, 45%, 50%, 55%, 60%, 65%, 70%, 75%, 80%, 85%, 90%, 95%, 96%, 97%, 98%, 99%, or 100%.

In some embodiments, “the biopolymer particle is coated with” is taken to mean “the biopolymer particle is bound by”.

The skilled person will be aware of techniques that can be used to determine the surface area of the biopolymer particle that is coated with the fusion protein, for example by transmission electron microscopy or proteolytic cleavage of the fusion protein followed by protein quantification.

As described above, in addition to the biopolymer particle binding domain, the fusion protein of the invention also includes an EV binding domain. The purpose of the EV binding domain is to bind to some factor or antigen on the target EV, and to bind in such a way that the contact between the fusion protein and the EV is maintained during isolation of the biopolymer particle/EV complex isolation.

The factor or antigen to which the EV binding domain binds can be any factor or antigen located on the surface of the EV, or located within the membrane of the EV such that at least a portion of the factor or antigen is available for contact with the EV binding domain. In some embodiments the factor can be considered an antigen.

In some embodiments the EV binding domain binds to a factor or antigen that is not specific to a particular EV and is, for example, common across all, or a subset, or EVs. However, preferably the EV binding domain binds to a factor or antigen that is specific to a fraction of EVs in the sample, i.e. the target EVs which are to be isolated. Such target EVs may comprise, for example, therapeutic polypeptides located in the membrane In instances such as this, the therapeutic polypeptide may be the factor or antigen to which the EV binding domain binds, in which case the therapeutic polypeptide also serves as an “isolating antigen”. In other instances, the EV may comprise for example a therapeutic agent within the lumen of the vesicle and/or in the vesicle membrane, and an “isolating antigen” located in the membrane, the function of which is to simply bind to the EV binding domain of the fusion protein and allow isolation of the biopolymer particle/EV complex.

Examples of factors located in or on the EV membrane to which the EV binding domain can bind are described above.

Where the EV is to be used for therapeutic purposes, the EV binding domain may bind to a factor or antigen that is located in or on the EV due to cell engineering, for example where the EV has been produced in a cell that has been engineered to express a particular therapeutic polypeptide that is encapsulated by the EV, or wherein the therapeutic polypeptide becomes integrated into the EV membrane, or localised to the EV membrane surface. However, the methods, compositions and fusion proteins of the invention are also considered to have use in detecting naturally occurring EVs. For example, in some cancers, EVs are produced and released by the tumour cells which comprise particular tumour-specific polypeptides or antigens. In some embodiments therefore, the EV binding domain of the fusion protein of the invention is able to bind to such naturally occurring antigens. In these embodiments, the methods, compositions and fusion proteins have use in diagnostic methods. Accordingly, the invention also provides methods of isolating disease-associated or disease-specific exosomes from a sample obtained from a subject, wherein the method comprises the method of isolating extracellular vesicles from a sample according to the invention, and wherein the fusion protein comprises an extracellular vesicle binding domain that can bind to a disease-associated or disease-specific antigen located on the disease-associated or disease-specific extracellular vesicles. The invention also provides a method of diagnosing a disease in a subject or providing an indication that the subject likely has the disease, where the disease results in the production of disease-associated or disease-specific extracellular vesicles, wherein the method comprises isolating the disease-associated or disease-specific exosomes according to the method of the invention, and wherein where disease-associated or disease-specific extracellular vesicles are isolated, the subject is diagnosed with the disease or is determined to likely have the disease. In some embodiments the number or relative number of isolated disease-specific or disease-associated extracellular vesicles is quantified. The number of, or relative change in number of over time, of disease-specific or disease-associated EVs is considered to aid in diagnosis or prognosis.

Preferences for features of this and all embodiments are as defined elsewhere.

As described herein, the target EVs can be therapeutic EVs, for example may comprise a therapeutic factor located on the EV membrane, or inside the EV lumen. However, there are instances where the EV that is isolated is itself not inherently therapeutic or does not inherently have a particular function. Through the use of a particular arrangement of the fusion protein of the invention, which includes the presence of a site specific protease cleavage site and the specific cleavage of the site, a portion of the fusion protein can remain associated with the target EV. Where that portion has a particular function, or confers a particular property on the EV, the EV is said to be a functionalised EV. Typically, where a portion of the fusion protein remains associated with the EV, the EV will be functionalised since any portion of the fusion protein is expected to at least, for example, act as an antigen. In some embodiments, portions of the fusion protein that remain associated with the EV can have therapeutic properties themselves, or they can for example be used to target the functionalised EV to a particular cell or tissue type, for example. In these embodiments the lumen of the EV may comprise a particular therapeutic agent such as a small molecule, and the portion of the fusion protein that remains associated with the EV may be, for example, a ligand that associated with a receptor on a target cell surface, resulting in targeted drug administration. The invention also provides functionalised EVs according to the invention for use in medicine, and also provides methods of treatment that comprise administering a functionalised EV of the invention, for example a functionalised EV that has been made as described herein.

The functionalised EVs may have a role other than in therapy.

In some embodiments of the methods of the invention, the EV binding domain does not bind to a therapeutic antigen on the target EV, but instead may, for example, bind directly to the lipid membrane of the EV, for example in some situations the EV binding domain may be an anti-PE binding domain. Despite being a less targeted approach to isolating EVs, such an approach is considered to be useful. For example, Tim4 binds directly to phosphatidylserine (cell membrane phospholipid) displayed on the surface of EVs (see for example Nakai et al Scientific Reports 6: 33935). The skilled person will be aware of other approaches that can be used, for example wheat germ agglutinin binds to N-acetyl-D-glucosamine and Sialic acid which are cell-membrane components. Accordingly, in one embodiment the EV binding domain comprises a Tim4 phosphatidylserine binding domain or a wheat germ agglutinin domain. Other proteins that bind lipids/membrane components that can be incorporated into the fusion protein of the invention.

In preferred embodiments, the EV binding domain should be designed to specifically bind to the intended factor or antigen on the target EVs, for example bind to a particular protein antigen located in the EV membrane. In one embodiment this domain does not, or substantially does not bind to cellular components in the cell in which the fusion protein is produced, which may or may not be the same cell as that in which the biopolymer particle is produced.

In some instances, the EV binding domain does not bind directly to the EV or to a factor or antigen located in/on the EV, and instead binds to an intermediary molecule. For example, in some instances, the EV may be coated with streptavidin, for example by expression a CD63-streptavidin fusion protein, and the fusion protein may bind to the EV via a biotin intermediate.

Accordingly, in some embodiments, the EV binding domain facilitates the binding of the coated biopolymer particle to a further entity that is capable of binding specifically to an extracellular vesicle-specific surface antigen, for example. The EV binding domain in some embodiments binds to an intermediate factor that bridges the interaction between the fusion protein of the invention and the target EV.

In preferred embodiments, the EV binding domain binds directly to a factor or an antigen on the surface of the EV, or to the lipid(s) of the EV itself.

However, an advantage of the present invention is that it typically does not require engineering of the cell that produces the exosomes to isolate the exosomes.

The EV binding domain can be any type of protein domain capable of being expressed as a fusion protein. For example, the EV binding domain can be a protein fragment, a binding domain, a target-binding domain, a binding protein, a binding protein fragment, an affibody, an antibody, an antibody fragment, an antibody heavy chain, an antibody light chain, a single chain antibody, a single-domain antibody, a Fab antibody fragment, an Fc antibody fragment, an Fv antibody fragment, a F(ab′)2 antibody fragment, a Fab′ antibody fragment, a single-chain Fv (scFv) antibody fragment, a camelid antibody, an IgNAR Shark antibody, a DARPin, a nanobody, an antibody binding domain, an antigen, an antigenic determinant, an epitope, a hapten, an immunogen, an immunogen fragment, biotin, a biotin derivative, an avidin, a streptavidin, a substrate, an enzyme, an abzyme, a co-factor, a receptor, a receptor fragment, a receptor subunit, a receptor subunit fragment, a ligand, an inhibitor, a hormone, a lectin, a polyhistidine, a coupling domain, a DNA binding domain, a FLAG epitope, a cysteine residue, a library peptide, a reporter peptide, and an affinity purification peptide, or a combination thereof. Preferably the EV binding domain is an affibody.

In one embodiment the EV binding domain is not a camelid antibody or a camelid-derived antibody.

The skilled person will appreciate that by binding the fusion protein to the biopolymer particle, the coated particle can be isolated from the cell lysate or components by, for example, filtration or centrifugation to arrive at the required composition of coated biopolymer particles.

Once the coated biopolymer particles have been contacted to the sample of extracellular vesicles, it is often, though not always, useful to then separate the extracellular vesicles from the coated biopolymer particle. For example, if the extracellular vesicles are to be used in therapy, the association with the coated biopolymer particle may be unwanted.

As described above, the fusion protein comprises a sequence capable of being cleaved by a protease, optionally a site specific protease, optionally a TEV protease. This site can be positioned anywhere in the fusion protein, or within an associated linker, so that cleavage can occur at an appropriate position, for example leaving no or very little fusion protein remaining associated with the extracellular vesicle, or leaving larger portions of the fusion protein, for example functionalisation domains (see below) associated with the extracellular vesicle.

By using site specific cleavage, releasing the EVs from the coated biopolymer particle is simple, straightforward and gentle, avoiding the use of agents such as chelating agents which are required by prior art methods and which damage the EVs.

Self-cleaving modules such as a modified Sortase A (Srt A) from Staphylococcus aureus and its five amino acid recognition sequence can be incorporated into the fusion protein, which is considered to be a simple method to release the EVs (see for example Hay et al 2015 Microbial Cell Factories 14:190). The site-specific protease site may be anywhere in the fusion protein, for example may be in the biopolymer particle binding domain or may be in the EV binding domain. Accordingly, in one embodiment the fusion protein comprises a means of allowing a none of or a portion of the fusion protein to remain associated with the EV whilst allowing the biopolymer particle and particle-associated portion of the fusion protein to be released.

It will be apparent from the disclosure that the invention provides a method for functionalising the surface of extracellular vesicles, the method comprising:
(i) providing a composition of coated biopolymer particles, the biopolymer particles being coated with a fusion protein that comprises a functionalisation domain;
(ii) contacting the composition comprising the coated biopolymer particles with a sample comprising extracellular vesicles under conditions which allow the formation of a coated biopolymer particle-extracellular vesicle complex; and
(iii) processing the coated biopolymer particle-extracellular vesicle complex so as to provide a) a functionalisation domain-associated extracellular vesicle and b) a fusion protein portion-associated biopolymer particle.

Preferences for features of this and all embodiments are as defined elsewhere.

In order to create functionalised EVs, in those embodiments the coated biopolymer particle-extracellular vesicle complex must be processed so as to effectively split the complex into two parts, one in which the functionalisation domain remains associated with the EV, i.e. the functionalised EV, and the other which comprises the biopolymer particle that remains associated with the remaining portion of the fusion protein, which will typically include the biopolymer particle binding domain. The processing can be any processing which results in the complex being appropriately split. In some embodiments the processing requires that the fusion protein comprises means of allowing a portion, i.e. a “functionalisation domain” to remain associated with the EV whilst allowing the biopolymer particle and particle-associated portion of the fusion protein to be released. The skilled person will be aware of suitable means, at least one of which is the inclusion of a site-specific protease site, such as a site for the TEV protease, within the fusion protein. Self-cleaving modules such as a modified Sortase A (Srt A) from Staphylococcus aureus and its five amino acid recognition sequence can be incorporated into the fusion protein, which is considered to be a simple method to release the EVs (see for example Hay et al 2015 Microbial Cell Factories 14:190). The site-specific protease site may be anywhere in the fusion protein, for example may be in the biopolymer particle binding domain or may be in the EV binding domain. Accordingly, in one embodiment the fusion protein comprises a means of allowing a portion of the fusion protein to remain associated with the EV whilst allowing the biopolymer particle and particle-associated portion of the fusion protein to be released. In another embodiment the fusion protein comprises a site-specific protease site. Accordingly, in some embodiments the processing involves digestion of the complex with a site-specific protease.

As described above, the fusion protein and the interaction between the biopolymer particles, fusion protein and EVs can be used to “functionalise” the EVs, whilst at the same time releasing them from the coated biopolymer particle. In these instances, the fusion protein comprises a functionalisation domain. By functionalisation domain we include the meaning of any protein domain that can associate with the EV and remain associated with the EV following release of the biopolymer particle and remaining portion of fusion protein. For example, where the fusion protein comprises a site specific protease site, following cleavage, the functionalisation domain remains associated with the EV, for example on the surface of the EV whilst the biopolymer particle and remaining portion of the fusion protein are released.

Functionalisation domains can be used to add any function to an EV that can be conferred by a protein domain or domains. For example, the function conferred to the EV may be the ability to be recognised and bound by a particular molecule, such as an antibody. In this case the functionalisation domain is acting as an antigen. In other embodiments the function conferred is an enzymatic function, in which case the functionalisation domain comprises a domain that has enzymatic activity. In other embodiments the function conferred is a therapeutic function, in which case the functionalisation domain comprises a therapeutic function. In some embodiments the functionalisation domain is a membrane disrupting peptide or a cell targeting peptide.

In addition to the above means of producing functionalised EVs, the invention also provides means of providing functionalised biopolymer particles, for example functionalised bioplastic beads. In this instance the functionalisation domain remains associated with the biopolymer particle or bioplastic bead, and confers particular function(s) to the particle. The skilled person will understand that the preferences for this embodiment are as described herein, for example the fusion protein may comprise a site specific protease, and the coated biopolymer particle-extracellular vesicle complex may be processed by digesting the complex with a site-specific protease such as TEV.

Preferences for features of this and all embodiments are as defined elsewhere.

The skilled person will understand that fusion proteins often contain linker peptides between the various domains of the fusion protein. This linker is typically flexible and allows the domains to fold correctly and function independently of one another. Linker peptides are known to the skilled person. In some embodiments therefore the biopolymer particle binding domain is fused to the EV binding domain via a linker peptide. Where the fusion protein comprises a functionalisation domain, the biopolymer particle binding domain may be fused to the functionalisation domain via linker peptide. In the same or other embodiments the EV binding domain by be fused to the functionalisation domain via a linker peptide.

In some embodiments, the fusion protein comprises a linker polypeptide. In further embodiments the fusion protein comprises a linker polypeptide that comprises one or more site specific protease sites.

Linker peptides are known in the art. The inventors have found that longer linkers improve proteolytic release/surface labelling of the EVs. In some embodiments the linker peptide is more than 12 amino acids in length, for example more than 15, 20, 25, 30, 35, 40, 45, 50 60, 70, 80, 90, 100, 110, 120, 130, 140, 150 amino acids in length. The linker may comprise small, non-polar and/or small, polar amino acids. In particular embodiments the linker is 112 amino acid residues in length and comprises small, non-polar and/or small, polar amino acids.

As described above, the linker peptide may be a cleavable linker peptide, for example may comprise a site specific protease cleavage site, for example may comprise a TEV protease cleavage site.

As will be appreciated then, the fusion protein of the invention can take numerous forms and can comprise various different combinations of domains. The fusion protein has to comprise at least a biopolymer particle binding domain and a domain that either binds directly to the target EV, or is able to bind to an intermediate factor that bridges an interaction between the fusion protein of the invention and the target EV.

The biopolymer particle binding domain may be located at the N-terminus or at the C-terminus of the fusion protein. Likewise, the EV binding domain, or domain that binds to an intermediate factor that bridges the interaction between the fusion protein of the invention and the target EV can be located at the N-terminus or at the C-terminus.

As described herein, the fusion protein may also comprise a peptide linker. The fusion protein may also comprise a functionalisation domain. These domains can be located in any order. For example:

F=functionalisation domain (which may comprise a site specific protease site)
L=peptide linker (which may comprise a site specific protease site)
B=biopolymer particle binding domain (which may comprise a site specific protease site)
E=EV binding domain, or domain that binds to an intermediate factor that bridges the interaction between the fusion protein of the invention and the target EV (which may comprise a site specific protease site)

### BE

### BLE

BLFE (which can be used to create a functionalised EV)
BFLE (which can be used to create a functionalised biopolymer particle)

In some embodiments, the functionalisation domain F can be the same domain as the biopolymer particle binding domain B or the same domain as the EV binding domain, or domain that binds to an intermediate factor that bridges the interaction between the fusion protein of the invention and the target EV.

The fusion protein, for example any of domains FLBE can comprise a site specific protease site. Preferably where the fusion protein comprises a site specific protease site, it is located in the linker peptide.

As discussed herein, the biopolymer particle and the fusion protein can be produced independently, for example synthetically, and exposed to one another to form the coated biopolymer particle. However, as discussed herein, in a preferred embodiment, the biopolymer particle and the fusion protein are produced in the same cell.

As described above, preferences for features of the method of producing biopolymer particles coated with a fusion protein, such as the fusion protein, biopolymer, particle, cell etc are as defined elsewhere.

The skilled person will understand how to produce the biopolymer particle in a cell, and the necessary genes and constructs that may be required (see for example Kelwick et al 2015 PLOS One A forward-design approach to increase production of poly-3-hydroxybutyrate in genetically engineered Escherichia coli).

In one embodiment, the biopolymer particles, coated with the fusion protein, used in the method of isolating EVs of the invention are produced by a cell. For example, in one embodiment the coated biopolymer particles have been formed by a method comprising the steps of:
(i) providing a host cell that produces
   a) biopolymer particles; and
   b) a fusion protein capable of coating the biopolymer particles in the cells wherein the fusion protein comprises a biopolymer particle binding domain and an extracellular vesicle binding domain and a sequence capable of being cleaved by a protease, optionally a site specific protease, optionally a TEV protease;
(ii) cultivating the host cell under conditions suitable for the production of biopolymer particles coated with the fusion protein; and optionally
(iii) isolating the coated biopolymer particles from the host cell.

It will be clear that in this embodiment of producing the fusion protein coated biopolymer particle, the host cell produces both the biopolymer particles and the fusion protein, i.e. a single cell produces both of these components.

Accordingly, in one embodiment, the cell comprises:
i) A biopolymer particle production nucleic acid construct, and
ii) A fusion protein production nucleic acid construct.

As described above, the coated biopolymer particle of the invention may be coated by more than 1 different fusion protein. Accordingly, in some embodiments the host cell produces more than one fusion protein capable of coating the biopolymer particles in the cells. Preferences for the more than one fusion protein are as described in relation to other aspects and embodiments of the invention.

The skilled person is aware of the term nucleic acid construct, which encompasses linear and circular nucleic acid molecules. Typically the construct will be circular. The biopolymer production nucleic acid construct and the fusion protein production nucleic acid construct may be physically located on different nucleic acid molecules or may be located on the same nucleic acid molecule, for example may be located on the same or different plasmids. The biopolymer production nucleic acid construct fusion protein production nucleic acid construct are constructs that typically comprise one or more promoters, and typically comprise at least one open reading frame which, following transcription and translation results in one or more proteins required to produce the biopolymer particle, and the fusion protein. Where the biopolymer production nucleic acid construct and the fusion protein production nucleic acid construct are located on the same nucleic acid molecule, the open reading frames that encode the proteins necessary for the production of the biopolymer particle and the fusion protein open reading frame may be driven by the same promoter.

The skilled person will also appreciate that where the biopolymer production nucleic acid construct and the fusion protein production nucleic acid construct are located on different nucleic acid molecules, the promoter that drives expression from the open reading frames that encode the proteins necessary for the production of the biopolymer particle and the fusion protein may be the same type of promoter, for example may have the same sequence. However, where the promoters have the same sequence, it is not considered to be possible to differentially drive expression of the open reading frames that encode the proteins necessary for the production of the biopolymer particle and the fusion protein open reading frame. An advantage of the present invention is that it allows the relative production rates of the biopolymer particle and the associated fusion protein to be tailored to particular requirements. For example, difference in the relative expression levels of the biopolymer particle and the fusion protein can lead to an increased or decreased surface area of the particle being coated by the fusion protein. For example, it is desired to produce a biopolymer particle that has a high density of associated fusion protein, the relative expression levels the fusion protein may be increased, relative to the expression of the proteins required to produce the biopolymer particle. Conversely, where a lower density of fusion protein coating is required, the expression level of the proteins required to produce the biopolymer particle may be increased, relative to the expression of the fusion protein. Accordingly, in preferred embodiments, the promoter(s) that drive expression of the proteins required to make the biopolymer particle, and the promoter(s) that drive expression of the fusion protein, are different. In this way, expression from each protein can be controlled.

The skilled person will realise that the promoters described herein and for use in the present invention may be constitutive promoters, but that in some instance, such as those discussed above, inducible and/or repressible promoters are preferred.

In all aspects and embodiments, the promoters may be constitutive, inducible or repressible. The promoters in some embodiments may be selected from inducible promoters, a synthetic promoter, a viral promoter or a phage promoter.

Accordingly, in one embodiment, the host cell comprises:
i) A biopolymer particle production nucleic acid construct that comprises a first promoter, optionally a first inducible or repressible promoter, and
ii) A fusion protein production nucleic acid construct that comprises a second promoter, optionally a second inducible or repressible promoter,
   optionally wherein the first and second inducible or repressible promoter are induced or repressed by different inducers or repressors.

In some embodiments, the
i) biopolymer particle production nucleic acid construct that comprises a first promoter, optionally a first inducible or repressible promoter, and the
ii) fusion protein production nucleic acid construct that comprises a second promoter, optionally a second inducible or repressible promoter,
   are both part of the same nucleic acid molecule, for example are part of the same vector or plasmid or minichromosome.

As described above, the coated biopolymer particle of the invention may be coated by more than 1 different fusion protein. Accordingly, in some embodiments the host cell comprises more than 1 fusion protein production nucleic acid construct that comprises a second promoter Preferences for the more than one fusion protein are as described in relation to other aspects and embodiments of the invention.

As discussed above, an approach in which the first and second promoters are regulated by different regulators, for example are induced or repressed by different inducers or repressors, allows the expression of the biopolymer particle and the fusion protein to be independently controlled. This allows for, for example, control over the level of coating of the biopolymer particle by the fusion protein, as described above.

The skilled person will appreciate that such nucleic acids that are designed to express a particular protein or proteins typically take the form of a vector, such as a plasmid.

The production of biopolymers by cells is well known in the art. As described herein, in some embodiments, the biopolymer is a bioplastic which in some embodiments comprises one or more of a polyhydroxyalkanoate (PHA), a poly(L-lactide) (PLLA), a polyethylene (PE), a polystyrene (PS), or a polythioester (PTE). Accordingly, in one embodiment, the biopolymer production module is a nucleic acid capable of expressing the proteins necessary for the production of one or more of a polyhydroxyalkanoate (PHA), a poly(L-lactide) (PLLA), a polyethylene (PE), a polystyrene (PS), or a polythioester (PTE). The skilled person is aware of the required enzymes for the production of biopolymers such as one or more of a polyhydroxyalkanoate (PHA), a poly(L-lactide) (PLLA), a polyethylene (PE), a polystyrene (PS), or a polythioester (PTE). The production of each polymer requires core metabolic enzymes/pathways, for example glycolysis, and additional specific enzymes required to complete the pathways, for example:
Glycolysis and link reaction feed into PhaA, PhaB and PhaC (phaCAB operon) and are responsible for producing the PHA polymers (see for example Kelwick et al 2018 Synthetic Biology 3: ysy016; Kelwick et al 2015 PLOS ONE 10(2): e0117202);
Bacterial polythioesters (PTEs), see for example Wubbeler and Steinbuchel 2014 Curr Opin Biotechnol 29: 85-92)
Polystyrene (PS) and polyethylene (PE) are not bioplastics but are considered to be biopolymers of the invention since they can be made biologically, see for example Lynch et al 2016 Biotechnology for Biofuels 9:3; McKenna and Nielsen 2011 Metab End 13: 544-554;
PLA see for example Jung et al 2010 Biotechnol Bioeng 105:161-171.

For example, where the biopolymer that is to be produced is a PHA, the biopolymer particle production nucleic acid can comprise a PHA production module that expresses a phaCAB operon. The phaCAB operon is well known to the skilled person and comprises three genes which are: a) phaC, which encodes the polyhydroxyalkanoate (PHA) synthase; b) phaA, which encodes a 3-ketothiolase; c) and phaB, which encodes an acetoacetyl coenzyme A (acetoacetyl-CoA) reductase. These three genes may be found in the operon in any order.

It will also be apparent to the skilled person that any genes that are found as part of an operon need not be expressed as part of an operon. For example, the cell that is to produce a PHA biopolymer particle may comprise a phaC gene driven by for example promoter A, a phaA gene driven by a promoter B, and a phaB gene driven by a promoter C, and which may all be located on different nucleic acid molecules.

Accordingly, it is not considered to be important how the genes are expressed or what molecule they are expressed from, provided that they are capable of acting together and producing the biopolymer particle.

In some embodiments the biopolymer particle production nucleic acid construct comprises a PHA production module that expresses a phaCAB operon, optionally comprising:
i) a constitutive promoter;
a ribosome binding site such as a synthetic or natural ribosome binding site linked to a polynucleotide encoding a phaC gene; and/or
a synthetic or natural ribosome binding sites linked to a polynucleotide encoding a phaA gene and a polynucleotide encoding a phaB gene;
   and
ii) A module that expresses the fusion protein, optionally wherein the module comprises a promoter, optionally an inducible promotor or a constitutive promoter and a polynucleotide encoding the fusion protein, optionally wherein the module comprises a synthetic ribosome binding site.

In some embodiments the ribosome binding site linked to a polynucleotide encoding a phaC gene is a synthetic ribosome binding site and the ribosome binding site linked to a polynucleotide encoding a phaA gene and a phaB gene is a natural ribosome binding site.

The host cell may be any cell capable of expressing the required constructs and polypeptides and capable of forming biopolymer particles.

In some embodiments the host cell is: a bacterial cell, for example a cyanobacterial cell; an archaeal cell, for example a haloarchaeal cell; a fungal cell, for example a yeast cell; or a plant cell.

In some embodiments the bacterial cell is selected from the genera Alcaligenes, Azotobacter, Bacillus, Chlorogloea, Cupriavidus, Escherichia, Gloeothece, Haloferax, Halomonas, Lactobacillus, Pseudomonas, Raistonia, Spirulina, Synechococcus, or Thermus.

In the same or alternative embodiments the host cell is a bacterial cell selected from the group comprising Alcaligenes latus, Azotobacter chroococcum, Azotobacter vinelandii, Bacillus amyloliquefaciens DSM7, Bacillus laterosporus, Bacillus licheniformis, Bacillus macerans, Bacillus cereus, Bacillus circulans, Bacillus firmus G2, Bacillus subtilis I68, Bacillus subtilis K8, Bacillus sphaericus X3, Bacillus megaterium Y6, Bacillus coagulans, Bacillus brevis, Bacillus sphaericus ATCC 14577, Bacillus thuringiensis, Bacillus mycoides RLJ B-017, Bacillus sp. JMa5, Bacillus sp. INT005, Chlorogloea fitschii, Cupriavidus necator, Escherichia coli, Haloferax mediterraneis, Halomonas elongate, Halomonas species TD01, Halomonas sp. KM-1, Halomonas smyrnensis, Halomonas profundus, Pseudomonas aeruginosa, Pseudomonas mendocina PSU, Pseudomonas oleovorans, Pseudomonas putida, Ralstonia eutropha, or Thermus thermophilus.

In some embodiments, where the host cell is a yeast cell, the yeast cell is a Saccharomyces cerevisiae or Pichia pastoris cell. The host cell may be a fungal cell that is for example a Fusarium solani Thom cell. The host cell may be a plant cell that is for example an Arabidopsis thaliana, Camelina sativa, Nicotiana tabacum or Saccharum officinarum cell.

The host cell may produce any number of coated biopolymer particles. In some embodiments, the host cell comprises:
a) between about 5 and about 60 coated biopolymer particles, for example between about 10 and about 50, about 20 and about 40, about 30;
b) at least about 5 coated biopolymer particles, at least about 10, 20, 30, 40, 50 or at least about 60; and/or
c) less than about 60 coated biopolymer particles, less than about 50, 40, 30, 20, or less than about 5.

In some embodiments the host cell comprises 32 coated biopolymer particles.

In some embodiments the host cell comprises at least 5 coated biopolymer particles.

The coated biopolymer particles may be isolated from the host cell by any means. The skilled person will be aware of suitable means. For example in one embodiment the biopolymer particles are isolated from the host cell by disrupting the cell and separating the particles. In some instances, disrupting the cell is performed by physical disruption, such as by sonication, a cell press, detergent lysis, freeze-thawing, bead-beating, hypotonic cell disruption, or enzymatic disruption.

In other embodiments, isolating the biopolymer particles from the host cell is performed using a cell sorter, centrifugation, gravity sedimentation, electrophoresis, filtration, size exclusion chromatography or affinity chromatography. In preferred embodiments, the biopolymer particles are isolated from the cell by filtration.

As described above, in addition to providing a method of isolating extracellular vesicles from a sample, the invention also provides a method of preparing coated biopolymer particles, where the biopolymer particles are coated with a fusion protein. In one embodiment the method of preparing coated biopolymer particles involves:
(i) providing a host cell that produces
   a) biopolymer particles; and
   b) a fusion protein capable of coating the biopolymer particles in the cells wherein the fusion protein comprises a biopolymer particle binding domain and an extracellular vesicle binding domain and a sequence capable of being cleaved by a protease, optionally a site specific protease, optionally a TEV protease;
(ii) cultivating the host cell under conditions suitable for the production of biopolymer particles coated with the fusion protein; and optionally
(iii) isolating the coated biopolymer particles from the host cell.

Preferences for this aspect of the invention are as defined elsewhere, for example the preferences for the cell, the particles, fusion protein and nucleic acid construct, isolation method. For example, one embodiment provides a method of preparing coated biopolymers, wherein the biopolymer is a PHA based biopolymer and has been made by an E. coli cell expressing a phaCAB operon from a first inducible promoter, wherein the same E. coli cell also expresses a fusion protein from a second inducible promoter, wherein the fusion protein comprises a functionalisation domain that targets exosomes to a particular type of tumour cell (F), a peptide linker (L) that comprises a site specific protease site, a biopolymer particle binding domain (B) that is IbpA and an EV binding domain (E) that is an affibody that binds to an antigen on the target exosome. In an additional embodiment the biopolymer particle is coated with a second fusion protein wherein the second fusion protein does not comprise a functionalisation domain.

The invention also provides a cell or host cell as described herein, for example a cell that comprises i) A biopolymer particle production nucleic acid construct, and
ii) At least one fusion protein production nucleic acid construct
   optionally wherein (i) and (ii) above are part of the same nucleic acid molecule, for example part of the same vector, plasmid or minichromosome.

Preferences for the cell, for example type of host cell, biopolymer particle production nucleic acid construct and the fusion protein production nucleic acid construct as defined herein.

The skilled person will also realise that the invention provides one or more nucleic acid constructs that are suitable for use with the present invention. For example, the invention provides:
i) A biopolymer particle production nucleic acid construct, and/or
ii) A fusion protein production nucleic acid construct.

Both (i) and (ii) above may be located on the same single nucleic acid molecule, or may be on separate molecules. In addition, one or both, or the single nucleic acid molecule that comprises (i) and (ii) may comprise more than one fusion protein production nucleic acid constructs, for example that is suitable for use in producing a coated biopolymer particle that is coated with more than one fusion protein. Preferences for the more than one fusion protein are as described herein.

The invention also provides a nucleic acid encoding the fusion protein of the invention, i.e. with or without associated regulatory elements such as a promoter.

The invention also provides an expression construct comprising:
i) a nucleic acid encoding a fusion protein as defined herein:
ii) a nucleic acid encoding a further entity that is capable of binding to an extracellular vesicle-specific surface antigen.

In some embodiments the expression construct also comprises a nucleic acid encoding a biopolymer synthase, for example wherein one or more of the nucleic acids that encode the fusion protein, the further entity and the biopolymer synthase are operably linked to at least one promoter.

The invention also provides the following nucleic acid constructs, as described in the Examples:

### [1] PBD-AffiEGFR1907, [2] PBD-AffiZHER2-342, [3] PBD-HEP, [4] HspA-AffiEGFR1907, [5] HspA-AffiZHER2-342, [6] HspA-HEP, [7] PBD-HIS-AffiEGFR1907 and [8] PBD-AffiEGFR1907-HIS.

Preferences for the various nucleic acid constructs of the invention are as described herein. For example, as described above, the fusion protein production nucleic acid construct may be arranged so as to produce a fusion protein that comprises a functionalisation domain.

It will also be appreciated that the coated biopolymer particles of the invention may be produced by a cell-free method. Accordingly, in one embodiment of the method of isolating extracellular vesicles from a sample, the coated bioparticles have been produced by a cell-free method. The invention also provides cell-free methods for producing the coated biopolymer particles. In some instances the cell-free method of producing coated biopolymer particles comprises the steps of
(i) providing a solution comprising
   a) at least one biopolymer particle production nucleic acid construct, that comprises a first promoter, optionally a first inducible or repressible promoter suitable for the production of biopolymer particles; and
   b) at least one fusion protein production nucleic acid construct that comprises a second promoter, optionally a second inducible or repressible promoter
(ii) maintaining the solution under conditions suitable for expression of the at least one biopolymer particle production nucleic acid construct and the at least one fusion protein production nucleic acid construct that comprises a second promoter, and for formation of biopolymer particles coated with the fusion protein; and optionally
(iii) isolating the coated biopolymer particles from the solution.

Preferences for this embodiment are as defined for other aspects and embodiments.

In other embodiments, the coated biopolymer particle may be formed by a method comprising the steps of:
(i) providing a biopolymer particle, for example a particle as described here;
(ii) providing one or more fusion proteins capable of coating the biopolymer particle; and
(iii) contacting the biopolymer particle with the one or more fusion proteins under conditions suitable for formation of biopolymer particles coated with the fusion protein.

Preferences for this embodiment are as defined for other aspects and embodiments.

Typically, the coated biopolymer particles will be provided as part of a composition, which comprises, for example, a liquid such as a buffer which is suitable for allowing and maintaining association between the biopolymer particle and the fusion protein. Accordingly, the invention provides a composition comprising one or more coated biopolymer particles. Preferences for the coated biopolymer particles are as described in relation to other embodiments and aspects.

The invention provides a fusion protein of the invention, as defined here, and also provides a composition comprising a fusion protein of the invention.

As described above, the coated biopolymer particles are contacted with a sample comprising EVs. This contacting step can be performed under any suitable conditions. The skilled person will be aware of suitable conditions to allow the fusion protein that coats the particle to bind to and maintain association with the EV. For example, in some embodiments the contacting of the composition comprising the coated biopolymer particles with a sample comprising extracellular vesicles, occurs:

a) in aqueous solution;
b) at a temperature between 4° C.-60° C.; and/or
c) at a pH between 6.0-8.5.

In some embodiments, for example where the fusion protein and/or the antigen on the EV to which the fusion protein binds comprise disulphide bonds, the contacting is performed under oxidising conditions to maintain the disulphide bonds.

The skilled person will appreciate that numerous embodiments of the invention lend themselves to being provided as a kit, or a kit of parts. For example, in one embodiment the invention provides a kit comprising:
i) an expression construct comprising a biopolymer production module, optionally wherein the biopolymer production module produces one or more of a polyhydroxyalkanoate (PHA), a poly(L-lactide) (PLLA), a polyethylene (PE), a polystyrene (PS), or a polythioester (PTE),
optionally wherein the biopolymer production module is a PHA production module that expresses a phaCAB operon, optionally comprising
a constitutive promoter,
a synthetic ribosome binding site linked to a polynucleotide encoding a phaC gene, and/or
natural ribosome binding sites linked to a polynucleotide encoding a phaA gene and a polynucleotide encoding a phaB gene;
ii) a nucleic acid according to the invention;
iii) an expression vector according to the invention;
iv) an expression construct encoding a fusion protein of the invention;
v) a fusion protein of the invention;
vi) a biopolymer particle or particles;
vii) a coated biopolymer particle or particles of the invention;
viii) an expression construct encoding a further entity that is capable of binding to an extracellular vesicle-specific surface antigen; and/or
ix) a host cell according to the invention.

It will be clear that the invention also provides a coated biopolymer particle, as described herein. Preferences for all features of the coated biopolymer particle, including preferred methods of forming the coated biopolymer particle are as defined herein.

For example, the invention provides a biopolymer particle coated with:
i) one or more fusion proteins according to the invention; or
ii) one or more fusion proteins according to the invention and further comprising the further entity that is capable of binding specifically to an extracellular vesicle-specific surface antigen.

It will be clear that the invention provides a composition comprising isolated EVs such as isolated exosomes, where the EVs such as exosomes have been isolated according to any of the methods described herein.

As described above, various aspects and embodiments lend themselves to therapeutic or diagnostic methods.

In one embodiment of a therapeutic use of the composition of the invention, the invention provides a composition comprising isolated EVs such as isolated exosomes, for example where the EVs such as exosomes have been isolated according to any of the methods described herein, for use in therapy.

In other examples the composition comprising isolated EVs such as isolated exosomes, for example where the EVs such as exosomes have been isolated according to any of the methods described herein can be used in cosmetic treatments or therapies.

Accordingly the invention provides therapeutic and non-therapeutic uses of the isolated EVs such as isolated exosomes, for example isolated EVs that have been isolated according to the methods of the invention.

In some embodiments the isolated EVs of the invention are functionalised EVs such as functionalised exosomes wherein the EV such as an exosome has been isolated using a fusion protein that comprised a functionalisation domain and wherein the functionalisation domain remains associated with the EV or the exosome. Preferences for features of this aspect of the invention are as defined herein. For example the functionalisation domain may be a protein domain that targets the EV such as the exosome to, for example, a cancer cell.

The isolated EVs of the invention have many medical and non-medical uses. For example, EVs such as exosomes can be used as cosmeceuticals to help with skin conditions/treat burns e.g. https://kimeralabs.com/products.

EVs such as exosomes can also be used as sources of biomarkers e.g. EVs isolated from patient blood samples may be used to indicate diseases such as cancer.

Patent derived Exosomes can be used as sources of biomarkers for companion diagnostics to monitor treatment e.g. whether tumour cells are responding to cancer treatments.

Exosomes can be isolated to be used as standards for diagnostic or therapeutic development.

In some embodiments, removal of cancer EVs from blood can be used in method of minimising metastasis and/or tumour EV signalling.

Accordingly, in one embodiment, the invention provides a method of removing EVs from the blood of a subject, for example from the blood of a subject that has been diagnosed with cancer, as part of a method of treating cancer (for example by reducing tumour cell signalling) and/or preventing cancer metastasis. Such a method can involve, for example, a fusion protein according to the invention, or coated biopolymer particles according to the invention.

In one embodiment, the invention provides a fusion protein according to the invention or a coated biopolymer particle of the invention for use in a method of treating cancer (for example by reducing tumour cell signalling) and/or preventing cancer metastasis. In some embodiments of such methods the fusion protein of the invention may be immobilised, via the biopolymer particle, to a solid support such a column typically used in purification methods, and blood or plasma from the patient may be passed over the solid support. EVs with the target factor will bind to the fusion protein, i.e. to the solid support, and be removed from the blood or plasma, which can then be recirculated back to the patient. See for example the Hemopurifier by Aethlon Medical Inc https://www.aethlonmedical.com/the-hemopurifier/the-hemopurifier-in-cancer.

The invention also provides a composition of coated biopolymer particles wherein the biopolymer particles are coated with a fusion protein as described herein.

Such a composition of coated biopolymer particles also has therapeutic and non-therapeutic/cosmetic uses. For example, in some embodiments, the coated biopolymer particles can be used in methods of diagnosis, as described above, wherein the EV binding domain of the fusion protein is able to bind to a factor or antigen on a target exosome, and wherein recovery of the target exosome is indicative of a particular disease or infection. See for example Castellanos-Rizaldos et al 2018 Clin Cancer Res 15: 2944-2950; Roy et al 2018 J Extracell Vesicles 7: 1438720; An et al 2015 J Extracell Vesicles 4: 1; He et al 2018 Theranostics 8: 237-255.

The exosomes may also be functionalised by the methods of the present invention wherein the functionalisation domain acts as a reporter, for example as a reporter of a particular disease or infection. In some embodiments the functionalisation domain located on the EV such as the exosome produces a colour change upon contact with a target molecule, for example a target molecule that is indicative of a disease state or infection.

The invention also provides a composition of functionalised biopolymer particles, wherein the biopolymer particles have been functionalised as described herein. Such functionalise biopolymer particles of the invention have uses in, for example, EV isolation, protein purification, and agglutination assays.

The invention also provides a composition of isolated extracellular vesicles. The extracellular vesicles isolated by the present methods are considered to be inherently different to extracellular vesicles isolated by other methods since the vesicles are subjected to much less stress and denaturing conditions, and so are considered to be superior in the nativity of the state of the extracellular vesicles.

The invention provides extracellular vesicles isolated by any of the claimed methods.

Regarding the medical uses of the compositions and methods of the invention, the invention provides:

A method of therapeutic or non-therapeutic treatment of a subject wherein the subject is administered any one or more of:

a) an isolated EV of the invention, for example an isolated exosome of the invention, for example where the exosome is a functionalised exosome;
b) a composition of isolated EVs of the invention, for example a composition of isolated exosomes of the invention, for example where the exosomes are functionalised exosomes;
c) a coated biopolymer particle of the invention, for example a coated bioplastic bead of the invention, for example where the bioplastic bead is functionalised according to the invention;
d) a composition of coated biopolymer particles of the invention, for example a composition of coated bioplastic beads of the invention, for example where the bioplastic beads are functionalised according to the invention;
e) a fusion protein according to the invention, for example where the fusion protein comprises a site specific protease site and a functionalisation domain;
f) a composition comprising a fusion protein according to the invention, for example where the fusion protein comprises a site specific protease site and a functionalisation domain.

The invention also provides:

a) an isolated EV of the invention, for example an isolated exosome of the invention, for example where the exosome is a functionalised exosome;
b) a composition of isolated EVs of the invention, for example a composition of isolated exosomes of the invention, for example where the exosomes are functionalised exosomes;
c) a coated biopolymer particle of the invention, for example a coated bioplastic bead of the invention, for example where the bioplastic bead is functionalised according to the invention;
d) a composition of coated biopolymer particles of the invention, for example a composition of coated bioplastic beads of the invention, for example where the bioplastic beads are functionalised according to the invention;
e) a fusion protein according to the invention, for example where the fusion protein comprises a site specific protease site and a functionalisation domain;
f) a composition comprising a fusion protein according to the invention, for example where the fusion protein comprises a site specific protease site and a functionalisation domain;
for use in a method of therapy, for example as described herein.

The invention also provides the use of

a) an isolated EV of the invention, for example an isolated exosome of the invention, for example where the exosome is a functionalised exosome;
b) a composition of isolated EVs of the invention, for example a composition of isolated exosomes of the invention, for example where the exosomes are functionalised exosomes;
c) a coated biopolymer particle of the invention, for example a coated bioplastic bead of the invention, for example where the bioplastic bead is functionalised according to the invention;
d) a composition of coated biopolymer particles of the invention, for example a composition of coated bioplastic beads of the invention, for example where the bioplastic beads are functionalised according to the invention;
e) a fusion protein according to the invention, for example where the fusion protein comprises a site specific protease site and a functionalisation domain; and/or
f) a composition comprising a fusion protein according to the invention, for example where the fusion protein comprises a site specific protease site and a functionalisation domain
for therapeutic treatment, for example treatment of diseases as disclosed herein.

The invention also provides methods of producing a fusion protein as described herein, i.e. a fusion protein that comprises a biopolymer particle binding domain and an extracellular vesicle binding domain and a sequence capable of being cleaved by a protease, optionally a site specific protease, optionally a TEV protease. Such fusion proteins may bind to biopolymer particles formed in the same cell as the fusion protein; or, the fusion proteins may be isolated from the cell, or produced synthetically, and used to coat, for example, an array that comprises an appropriate biopolymer. In this way, an array can be produced that is coated in the fusion protein described herein, which can then be used to capture EVs.

The invention provides a cell that expresses a fusion protein as described herein, but does not express a biopolymer particle as described herein.

The invention provides a fusion protein as described herein, and provides a composition comprising a fusion protein as described herein.

The invention also provides arrays which comprise a biopolymer of the invention and a fusion protein of the invention. This embodiment is similar to a coated biopolymer particle of the invention, but rather than a small particle, the solid substrate is in an array form. See for example 15.

In one embodiment the invention provides an array comprising a substrate that comprises a biopolymer as described herein coated with a fusion protein as defined herein, for example in one embodiment the array is a PLA film coated with a (PBD)-based fusion protein.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. For example, the invention provides a method for isolating oncosomes from a sample wherein the sample comprising the oncosomes is contacted with a bioplastic bead that has been coated, in vitro, with a fusion protein, wherein the fusion protein binds to the bioplastic bead via a PhaR-derived binding domain and wherein the fusion protein binds to a target factor or antigen on the oncosome via an affibody.

### FIGURE LEGENDS

FIG. 1—Examples of current techniques to isolate exosomes, along with advantages and disadvantages of each, compared to the methods of the present invention.

FIG. 2—Descriptions of the fusion protein constructs designed for the present invention. PBD is a PHA binding domain from PhaR. IbpA/HspA is an E. coli heat shock protein. 112L denotes a linker composed of 112 amino acids. TEV site refers to a proteolytic cleave site for the Tobacco Etch Virus (TEV) protease. Superfolder green fluorescent protein (sfGFP). AffiEGFR and AffiZHER 2 are epidermal growth factor receptor (EGFR) and human epidermal growth factor receptor 2 (HER2) binding Affibodies, respectively.

FIG. 3—A) Schematics of PBD-based exosome capture fusion protein designs. PBD is a PHA binding domain from PhaR. GFP is superfolder green fluorescent protein (sfGFP); B) Schematics of IbpA/HspA-based exosome capture fusion protein designs. IbpA/HspA is an E. coli heat shock protein. GFP is superfolder green fluorescent protein (sfGFP); C) Schematics of PBD-based exosome capture fusion protein designs that incorporate a HIS-tag. PBD is a PHA binding domain from PhaR.

FIG. 4—Dynamic Light Scattering (DLS) analysis of control (C104) and exosome-capture PHAs beads (1-8), n=3. The average sizes of the indicated PHAs beads are shown.

FIG. 5—HspA-HEP [6] functionalised PHAs beads [A] Flow cytometry analysis of non-functionalised control beads (C104) and three batches of HspA-HEP [6] beads that incorporate sfGFP as part of its design. [B] Visual inspection of batches of pelleted HspA-HEP [6] beads on a blue transilluminator.

FIG. 6—Analysis of HIS-tag surface display on exosome capture beads. [A] Fusion-protein schematics of constructs [7] PBD-HIS-AffiEGFR1907 and [8] PBD-AffiEGFR1907-HIS indicating the location of HIS-tags displayed on PHAs bead surface. [B] Flow cytometry analysis of unlabeled and labeled (Anti-HIS-PE) PHAs beads. Comparison of constructs [7] and [8] against non-functionalised control beads (C104), n=3, *p<0.05, ***p<0.0001.

FIG. 7—Flow cytometry analysis of captured extracellular vesicles. [A] Generic schematic of exosome capture beads and EV antibody labelling. [B] Control (C104) and exosome capture beads were incubated with HEK293 cell conditioned media and then stained with either control (IgG-PE) antibody or a cocktail of EV surface marker targeting antibodies (Anti-CD9, CD63 and CD81-PE). Antibody labelled samples were analysed on an Attune flow cytometer (YL1-A, Excitation 561 nm/Emission 578 nm), n=3.

FIG. 8—TEV removes sfGFP from fusion protein functionalised PHAs beads. Visual inspection of batches of pelleted beads on a blue transilluminator.

FIG. 9 Analysis of ultracentrifugation and PHA bead captured EVs. [A] Dynamic Light Scattering (DLS) analysis of captured EVs. Exosome capture beads (PBD-HIS-AffiEGFR1907 [7]) were incubated with the indicated batches of HEK293 conditioned media and washed. AcTEV was used to release the EVs. These released EVs were subsequently analysed with Dynamic Light Scattering (DLS) to determine EV sizes (diameters [nm]). EVs isolated, using ultracentrifugation, from the same batch of HEK293 conditioned media were used for comparison. [B] Qubit analysis of PHA Capture and ultracentrifugation isolated EV samples. Qubit 3.0 (Invitrogen) and the Qubit protein assay (Invitrogen) were used to analyse protein concentration.

FIG. 10 qNano gold analysis of PHA captured EVs. [A] Histogram of analysed particle diameters and concentrations. n=1886 individual particles analysed. qNano gold (IZON Science) is based upon Tunable Resistive Pulse Sensing (TRPS) and enables single particle (EV) analysis. [B] qNano analysis parameters.

FIG. 11—Optimisation of Polyhydroxyalkanoates (PHAs) production in engineered Escherichia coli. [A] Plasmid map of the optimised C104 phaCAB biosynthetic operon. [B] Optimised C104 biosynthetic operon enhances P(3HB) and P(3HB-co-3HV) production in engineered E. coli. Comparison against E. coli engineered with the Native Cupriavidus necator phaCAB operon.

FIG. 12—Part 1 of 3—Schematics of PhaC-based exosome capture fusion protein designs. C104 is a control design where PhaC (PHA synthase) has not been engineered as a fusion protein. GFP is superfolder green fluorescent protein (sfGFP); Vn96 is heat shock binding peptide (including those that are EV-associated) and MT1-Af7p is an MMP14 binding peptide. Part 2 of 3—Dynamic Light Scattering (DLS) analysis of control (C104) and exosome-capture PHAs beads (PhaC fusions: MT1-Af7p and Vn96), n=3. The average sizes of the indicated PHAs beads are shown. Part 3 of 3—Flow cytometry analysis of captured extracellular vesicles. Control (C104) and exosome capture beads (PhaC fusions with MT1-Af7p or Vn96 peptides) were incubated with HEK293 cell conditioned media and then stained with an EV surface marker targeting antibody (Anti-CD63-PE). [Top left] Generic schematic of PhaC exosome/EV capture beads and EV antibody labelling. [Top right] Flow cytometry histogram of control (C104) and EV capture beads (PhaC fusions: MT1-Af7p and Vn96). [Bottom] Captured and antibody labelled samples were analysed on an Attune flow cytometer (GFP: BL1-A Excitation 488 nm/Emission 530-30; Anti-CD63 PE: YL1-A, Excitation 561 nm/Emission 578 nm), n=3.

FIG. 13—High-throughput workflow for capture of extracellular vesicles/exosomes. [A] Schematic of workflow. [B] Flow cytometry analysis of PHAs captured EVs from HEK293 conditioned media—sfGFP (Attune BL1-A Ex. 488 nm/Em. 530-30 nm) and cell mask orange (CMO) stained EVs (Attune YL1-A Ex. 561 nm/Em. 5 85-16 nm). Nile red (PHAs content measurement) was carried out on a BMG CLARIOstar (Ex. 560-15 nm/Em. 610-20. Columns 1 and 2—C104 control beads, columns 3 and 4 PhaC-MT1-Af7p beads, columns 5 and 6 PhaC-Vn96 beads, columns 7 and 8 pre-stained EVs only, columns 9 and 10 flow cytometry calibration beads (1 □m) and columns 11 and 12 PBS only.

FIG. 14—Optimisation of flexible amino acid linkers. [A] Schematics of different flexible amino acid linker lengths (12, 22 or 112 amino acids) within PhaC-fusion proteins. [B] Analysis of 12aa, 22aa and 112aa flexible linker control (C) and TEV site containing (T) PhaC-fusion protein designs. Functionalised PhaC-fusion PHAs beads were treated with units (10 U) of Tobacco Etch Virus (TEV) protease. Proteolytically released sfGFP in supernatant samples were analysed using a CLARIOstar plate reader (483-14 nm/530-nm) and these fluorescence data were normalised against untreated controls of the same PHAs bead batch. PHAs beads were analysed using flow cytometry and PHAs bead geometric mean (Attune BL1-A, 488 nm/530-30 nm) of TEV treated beads were normalised against untreated controls of the same biosensor batch. Error bars denote standard error of the mean, n=4-8, Student t-test *P<0.05, **P<0.01,****P<0.0001 or not statistically significant (ns).

FIG. 15—EV capture array. PLA films were coated with either control (PhaC from C104 operon) or PHAs Binding Domain (PBD)-based exosome capture fusion proteins [constructs 1, 2, 3 or 7]. Coated PLA films were incubated with HEK293 conditioned media and captured EVs were stained with either control (IgG-PE) antibody or a EV surface marker targeting antibody (Anti-CD81-PE). Coated PLA films and captured EVs were well scanned using a CLARIOstar plate reader (sfGFP: Ex. 483-14 nm/Em. 530-30 nm; PE: Ex. 496-15 nm/Em. 578-20 nm). Whole-well scanned data was averaged and displayed as a heatmap.

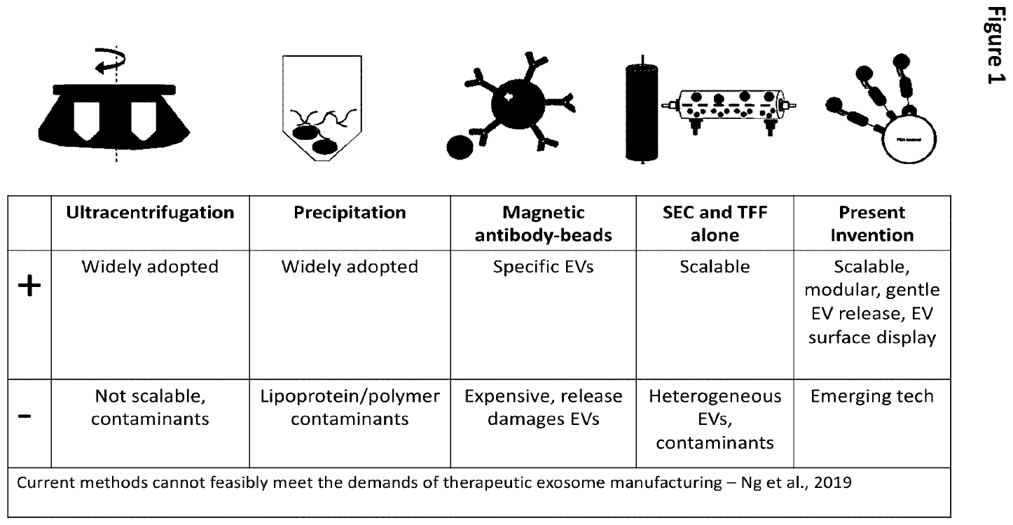

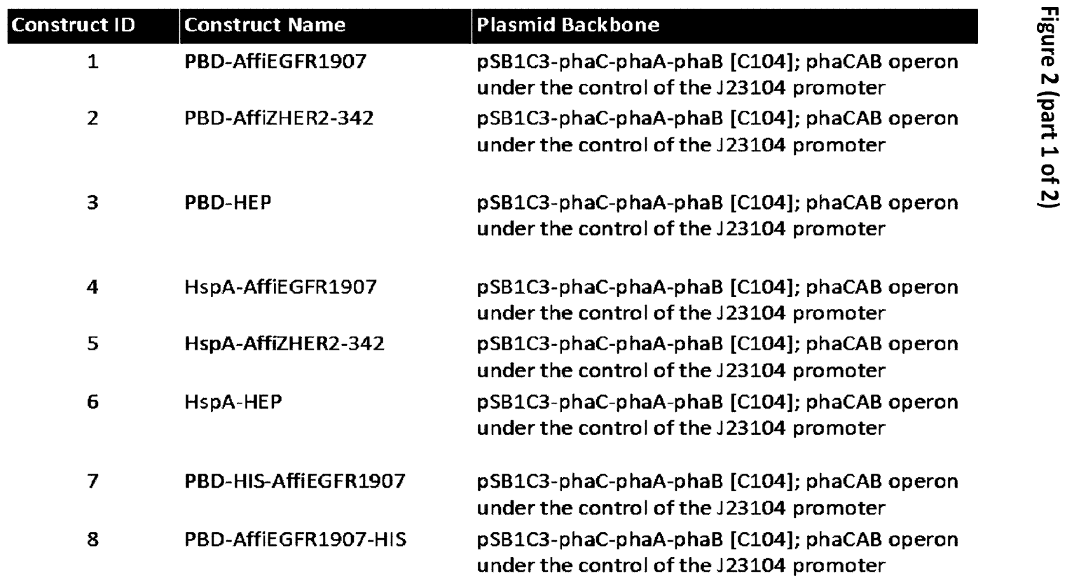

### REFERENCES

Armstrong, J. P. K., et al 2017. Re-Engineering Extracellular Vesicles as Smart Nanoscale Therapeutics. ACS Nano 11, 69-83. doi:10.1021/acsnano.6b07607
Bebelman, M. P., et al., 2018. Biogenesis and function of extracellular vesicles in cancer. Pharmacol. Ther. 188, 1-11. doi:10.1016/j.pharmthera.2018.02.013
Cheng, Y., et al., 2019. Effect of pH, temperature and freezing-thawing on quantity changes and cellular uptake of exosomes. Protein Cell 10, 295-299. doi:10.1007/s13238-018-0529-4
Colao, I. L., et al 2018. Manufacturing Exosomes: A Promising Therapeutic Platform. Trends Mol. Med. 24, 242-256. doi:10.1016/j.molmed.2018.01.006
Du, J., Rehm, B. H. A., 2018. Purification of therapeutic proteins mediated by in vivo polyester immobilized sortase. Biotechnol. Lett. 40, 369-373. doi:10.1007/s10529-017-2473-4
Gonzalez-Miro, M., et al 2019. Polyester as Antigen Carrier toward Particulate Vaccines. Biomacromolecules acs.biomac.9b00509. doi:10.1021/acs.biomac.9b00509
Kelwick, R., et al 2015. A Forward-Design Approach to Increase the Production of Poly-3-Hydroxybutyrate in Genetically Engineered Escherichia coli. PLoS One 10, e0117202. doi:10.1371/journal.pone.0117202
Kelwick, R., et al 2018. Cell-free prototyping strategies for enhancing the sustainable production of polyhydroxyalkanoates bioplastics. Synth. Biol. 3. doi:10.1093/synbio/ysy016
Kim, Y.-S., et al 2018. The potential theragnostic (diagnostic+therapeutic) application of exosomes in diverse biomedical fields. Korean J. Physiol. Pharmacol. 22, 113-125. doi:10.4196/kjpp.2018.22.2.113
Kim, S. M., et al 2017. Cancer-derived exosomes as a delivery platform of CRISPR/Cas9 confer cancer cell tropism-dependent targeting. J. Control. Release 266, 8-16. doi:10.1016/j.jconrel.2017.09.013
Konoshenko, M. Y., et al 2018. Isolation of Extracellular Vesicles: General Methodologies and Latest Trends. Biomed Res. Int. 2018, 1-27. doi:10.1155/2018/8545347
Lener, T., et al 2015. Applying extracellular vesicles based therapeutics in clinical trials—an ISEV position paper. J. Extracell. Vesicles 4, 30087. doi:10.3402/jev.v4.30087
Li, S., et al 2018. Exosomal cargo-loading and synthetic exosome-mimics as potential therapeutic tools. Acta Pharmacol. Sin. 39, 542-551. doi:10.1038/aps.2017.178
Ng, K. S., et al 2019. Bioprocess decision support tool for scalable manufacture of extracellular vesicles. Biotechnol. Bioeng. 116, 307-319. doi:10.1002/bit.26809
Patel, D. B., et al 2018. Towards rationally designed biomanufacturing of therapeutic extracellular vesicles: impact of the bioproduction microenvironment. Biotechnol. Adv. 36, 2051-2059. doi:10.1016/j.biotechadv.2018.09.001
Raposo, G., Stahl, P. D., 2019. Extracellular vesicles: a new communication paradigm?Nat. Rev. Mol. Cell Biol. 20, 509-510. doi:10.1038/s41580-019-0158-7
Roy, S., et al 2018. Extracellular vesicles: the growth as diagnostics and therapeutics; a survey. J. Extracell. Vesicles 7, 1438720. doi:10.1080/20013078.2018.1438720
Théry, C., et a 2018. Minimal information for studies of extracellular vesicles 2018 (MISEV2018): a position statement of the International Society for Extracellular Vesicles and update of the MISEV2014 guidelines. J. Extracell. Vesicles 7, 1535750. doi:10.1080/20013078.2018.1535750
Wang, J., et al 2017. Exosome-Based Cancer Therapy: Implication for Targeting Cancer Stem Cells. Front. Pharmacol. 7, 533. doi:10.3389/fphar.2016.00533
Watson, D. C., et al 2018. Scalable, cGMP-compatible purification of extracellular vesicles carrying bioactive human heterodimeric IL-15/lactadherin complexes. J. Extracell. Vesicles 7. doi:10.1080/20013078.2018.1442088
Wiklander, O. P. B., et al 2015. Extracellular vesicle in vivo biodistribution is determined by cell source, route of administration and targeting. J. Extracell. Vesicles 4, 26316. doi:10.3402/jev.v4.26316
Willis, G. R., et al 2017. Toward Exosome-Based Therapeutics: Isolation, Heterogeneity, and Fit-for-Purpose Potency. Front. Cardiovasc. Med. 4. doi:10.3389/fcvm.2017.00063
Zhang, Y., et a 2019. Exosomes: biogenesis, biologic function and clinical potential. Cell Biosci. 9, 19. doi:10.1186/s13578-019-0282-2
Zhang, D., et a 2018. Exosome-Mediated Small RNA Delivery: A Novel Therapeutic Approach for Inflammatory Lung Responses. Mol. Ther. 26, 2119-2130. doi:10.1016/j.ymthe.2018.06.007

### EXAMPLES

Development of Exosome Capture PHAs Beads

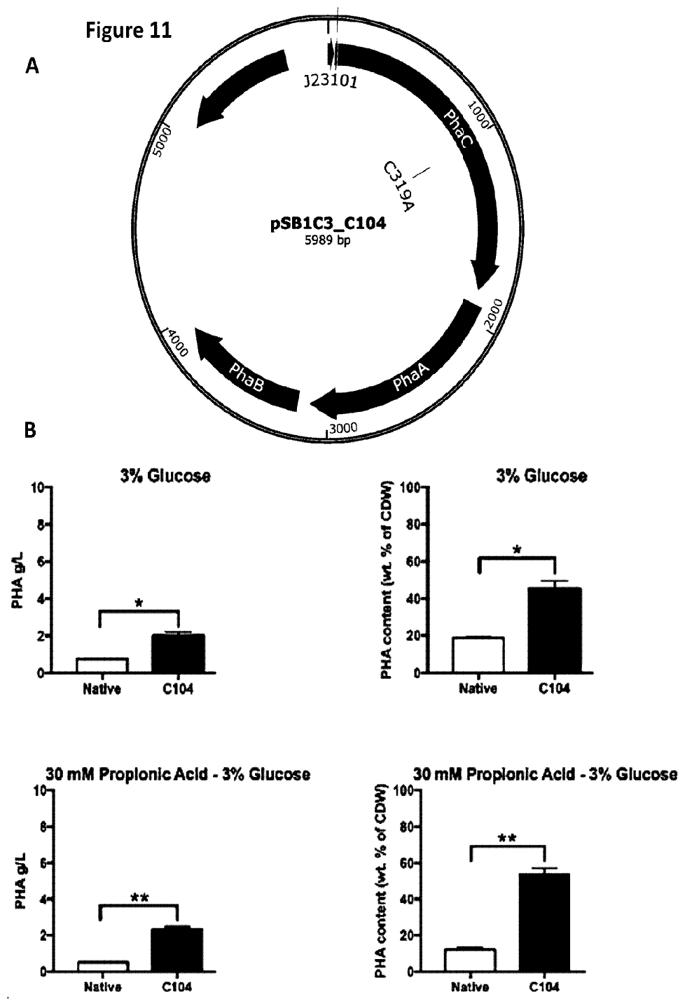

During our previous projects we used model-guided design and cell-free prototyping strategies to optimise the microbial production of PHAs-based biopolymers, at a range of production scales (Kelwick et al., 2015; 2018). These projects enabled us to engineer and develop phaCAB operons that produce relatively high levels of PHAs in phaCAB-engineered Escherichia coli (FIG. 11). Essentially, phaCAB-engineered E. coli convert Acetyl-CoA into poly-3-hydroxybutyrate (P(3HB)). Acetyl-CoA is enzymatically processed by PhaA (3-ketothiolase) to form acetoacetyl-CoA. Then, PhaB (acetoacetyl-CoA reductase) reduces acetoacetyl-CoA to form (R)-3-hydroxybutyl-CoA ((R)-3HB-CoA), which is finally polymerised by PhaC (PHA synthase) to form the final PHAs polymer—P(3HB) (Kelwick et al., 2015; 2018). Several studies have noted that PhaC remains bound to PHAs beads (Du et al., 2018; Gonzalez-Miro et al., 2019).

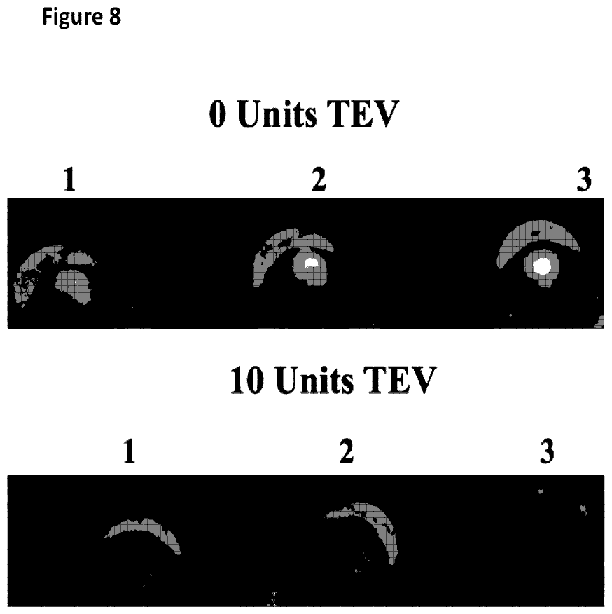

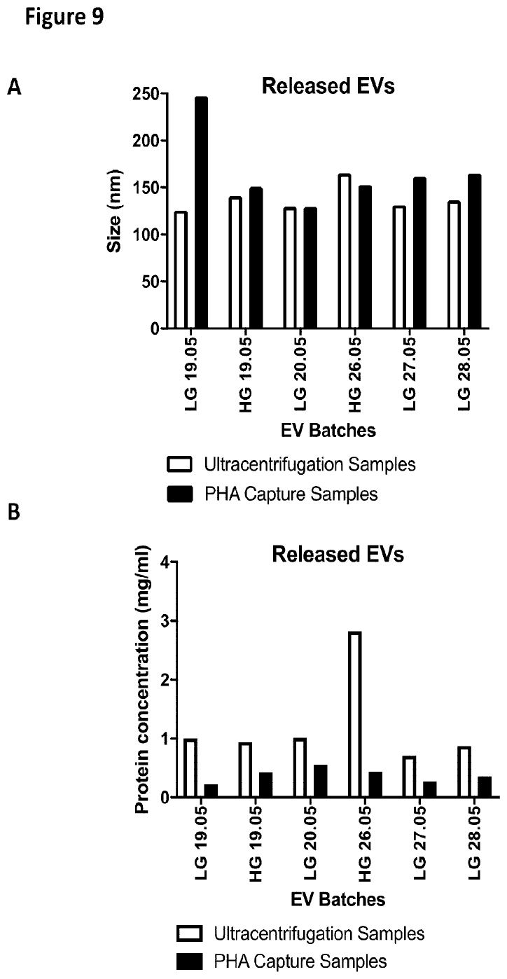

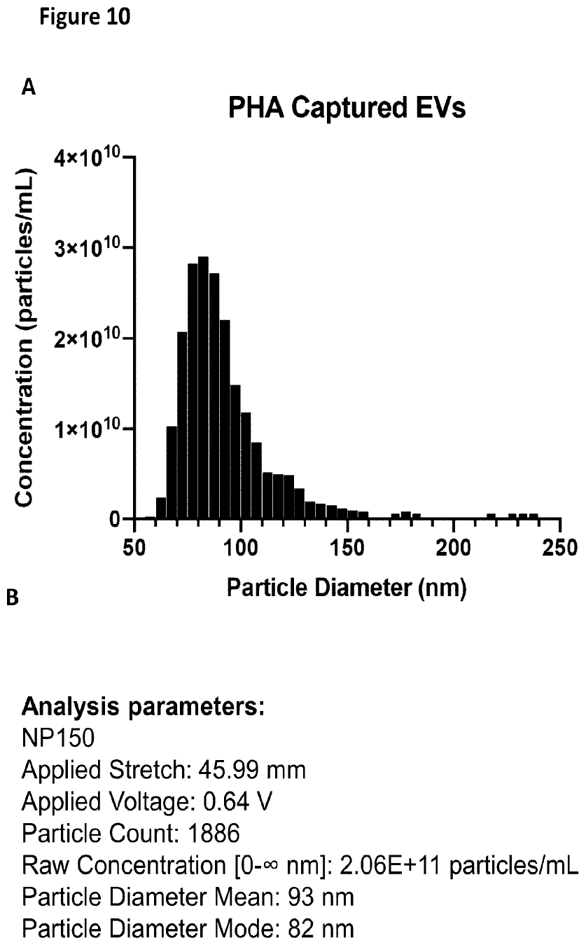

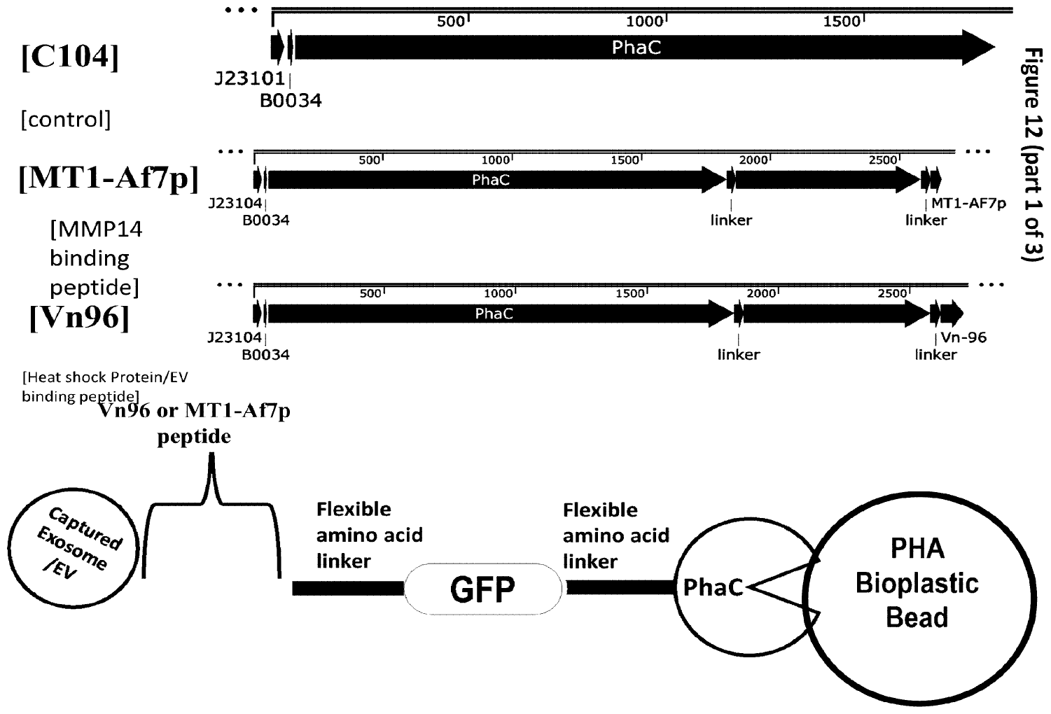

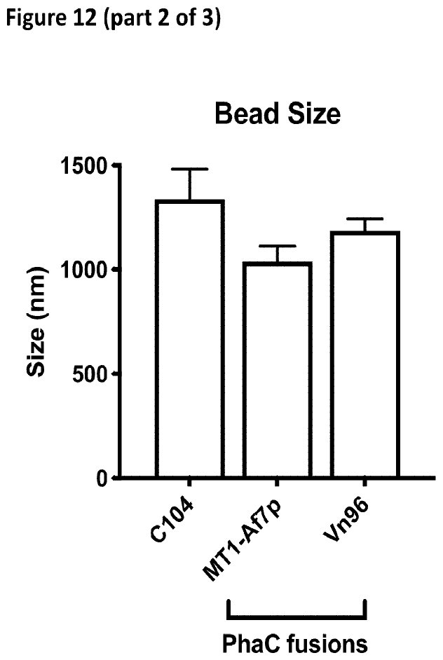

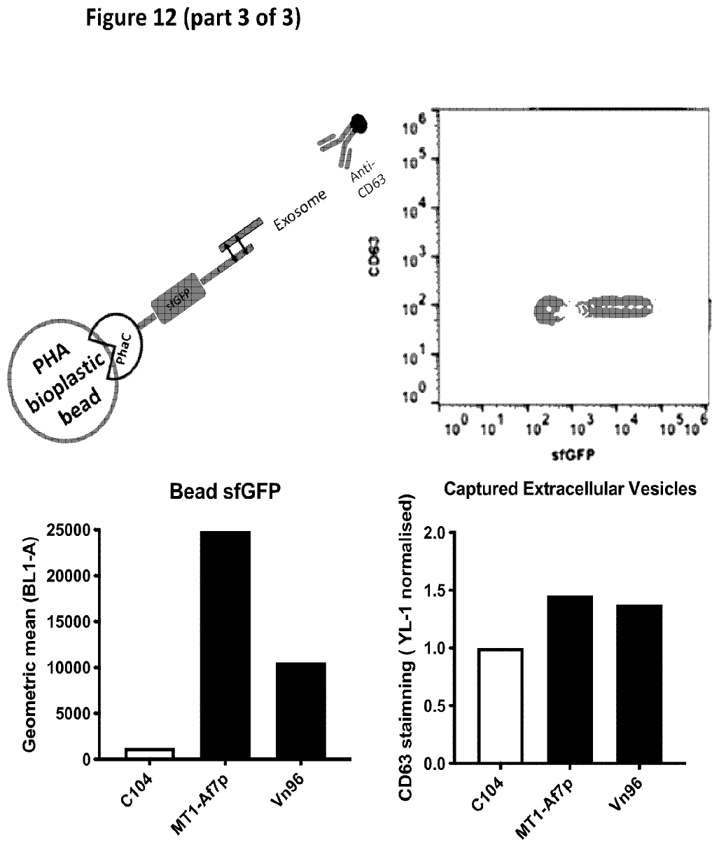

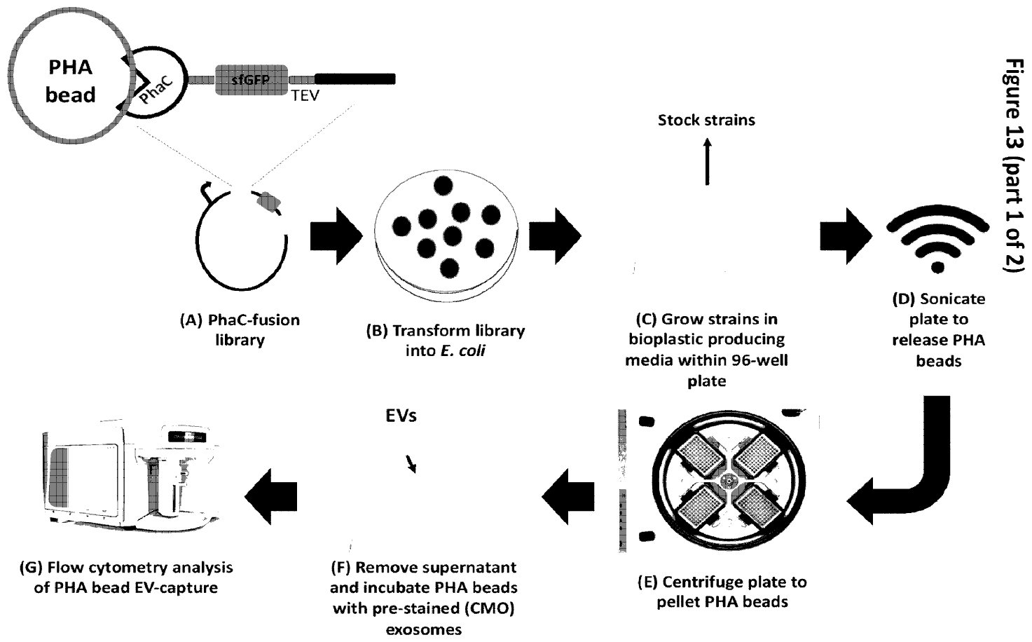

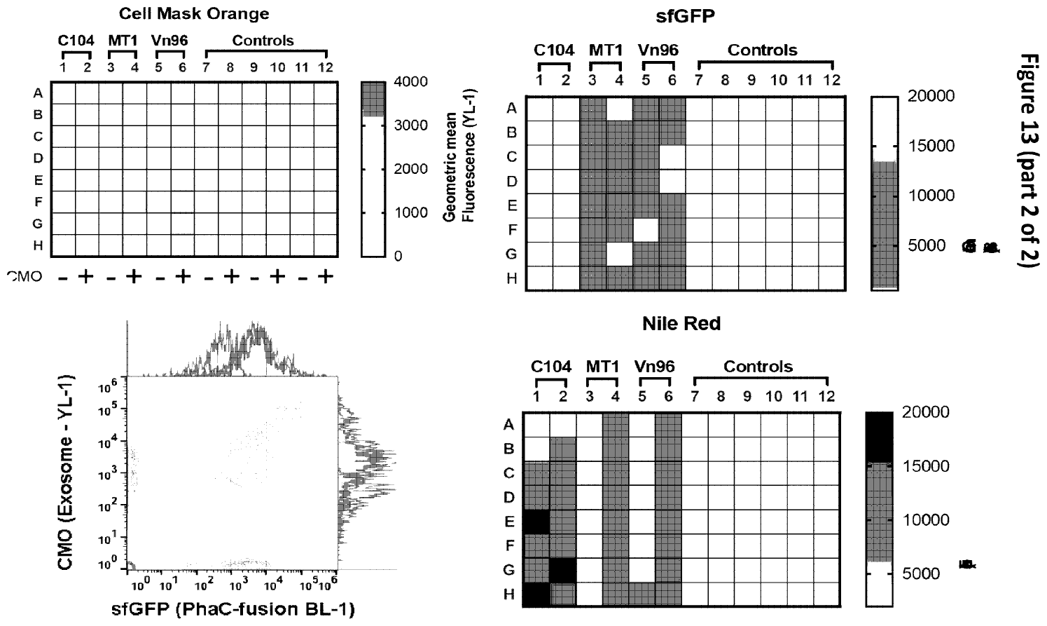

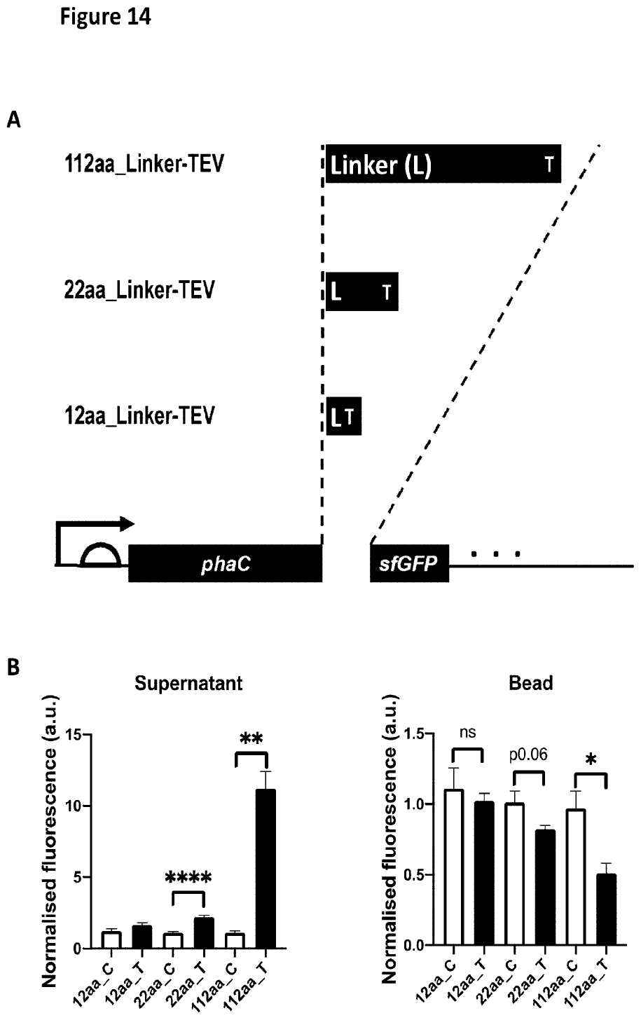

Initially, we engineered phaCAB operons that incorporated PhaC-fusion proteins that once produced would be capable of producing functionalised PHAs granules that could capture extracellular vesicles (including exosomes). These PhaC-fusion constructs included PhaC, a twelve amino acid linker, sfGFP, an additional twelve amino acid linker and either a heat shock binding peptide (Vn96; Ghosh et al., 2014) or an MMP14 binding peptide (MT1-Af7p; Zhu et al., 2011) (FIGS. 12-13). These PhaC-fusion were designed as IDT gblocks and then cloned into C104 vector and were termed C104-Vn96 and C104-MT1-Af7p (FIG. 12; Table 1). C104-Vn96 and C104-MT1-Af7p functionalised PHAs granules were produced in engineered E. coli and were analysed using Dynamic Light Scattering and were typically ˜1.2 μm in size (FIG. 12). These PhaC-fusion based extracellular vesicle-capture particles were incubated with HEK293 cell conditioned media and then stained with a PE-conjugated antibody that targeted an EV surface marker (CD81-PE). Flow cytometry analysis of these antibody stained, extracellular vesicle capture particles revealed that C104-Vn96 and C104-MT1-Af7p PHAs beads stained positive for EV markers, indicating EV capture (FIG. 12). These PhaC-fusion based extracellular vesicle-capture particles also captured cell mask orange (CMO) stained HEK293 EVs within a high-throughput plate-based assay (FIG. 13). Three phaCAB operons (C104) that incorporated PhaC-fusion constructs with optimised flexible amino acid linkers and Tobacco Etch Virus proteolytic recognition motifs were also cloned from IDT gblocks (FIG. 14). These constructs were used to optimise the proteolytic release of fusion protein and upstream components (e.g. captured EVs) from the PHAs granules (FIGS. 8 and 14).

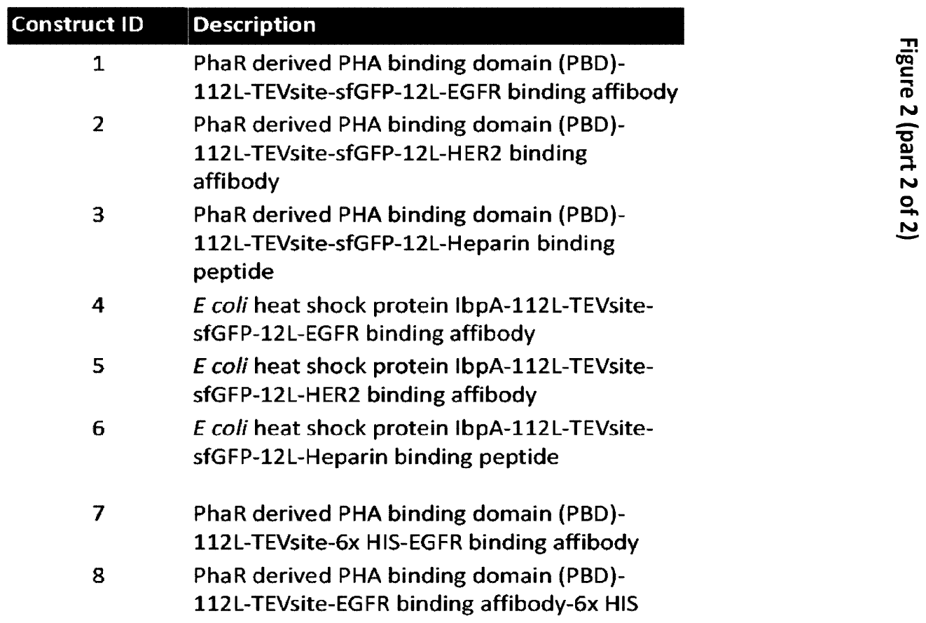

To develop an improved scalable exosome capture technology a novel strategy was devised that is optimised for extracellular vesicle (such as exosome) capture. These new constructs separate the phaCAB-operon from the extracellular vesicle-binding fusion protein, enabling their expression levels to be independently controlled and fine-tuned. This enables better control over the surface coating of the PHAs particles with the extracellular vesicle-binding fusion protein. The extracellular vesicle-binding fusion proteins can include either PhaR-derived binding domains (PBD [10.69 kDa], for example comprises or consists of SEQ ID NO: 2) or E. coli heat shock protein HspA (IbpA [16 kDa])-fusion proteins that also incorporate interchangeable (modular) extracellular vesicle-binding peptides or affibodies (FIG. 2). The PHAs-binding domains in these novel fusion proteins were engineered to be ˜4-6× smaller than PhaC (˜64.38 kDa), enabling greater coverage of the PHAs particles. Since, like PhaC, these fusion proteins remain bound to PHAs-based biopolymer particles, even post-purification, these fusion proteins can bind extracellular vesicles to their biopolymer particle. Thus, we can rapidly generate libraries of biopolymer extracellular vesicle-capture particles that are designed to capture specific extracellular vesicles, such as specific exosomes (FIG. 2, Table 1). Captured extracellular vesicles such as exosomes can then be processed using gravity sedimentation, low-speed centrifugation, flow cytometry and other methods. Alternatively, extracellular vesicles such as exosomes can be released without damaging them using an AcTEV protease (or metalloproteinase) to cleave-off the exosome from the biopolymer particle. Since we can design where the protease cleavage is positioned this also enables us to display an engineered peptide or protein (e.g. sfGFP, His-tag or cell targeting peptide) on the surface of the extracellular vesicle, such as an exosome.

TABLE 1
strain table

| Strain | Relevant features | Reference |
|---|---|---|
| JM109 | endA1, recA1, gyrA96, thi, hsdR17 (rₖ⁻, mₖ⁺), | Promega UK |
| | relA1, supE44, Δ(lac-proAB), [F′ traD36, proAB, | |
| | laqI^{q}ZΔM15] | |
| EV104 | JM109 pSB1C3 [EV104]; J23104 promoter and B0034 | Kelwick et al., |
| | RBS; BBaK608002; CamR | 2015 |
| C104 | JM109 pSB1C3-phaC-phaA-phaB [C104]; phaCAB | Kelwick et al., |
| | operon under the control of the J23104 promoter; CamR | 2015 |
| C104-Vn96 | JM109 pSB1C3-phaC_fusion-phaA-phaB [C104]; | This study |
| | phaCAB operon under the control of the J23104 | |
| | promoter; PhaC fusion: PhaC, 12aa Linker, sfGFP, 12aa | |
| | Linker, and Vn96 peptide; CamR | |
| C104-MT1-Af7p | JM109 pSB1C3-phaC_fusion-phaA-phaB [C104]; | This study |
| | phaCAB operon under the control of the J23104 | |
| | promoter; PhaC fusion: PhaC, 12aa Linker, sfGFP, 12aa | |
| | Linker, and MMP14-binding peptide-MT1-Af7p; CamR | |
| C104-12aa | JM109 pSB1C3-phaC_fusion-phaA-phaB [C104]; | This study |
| | phaCAB operon under the control of the J23104 | |
| | promoter; PhaC fusion: PhaC, 12aa Linker, TEV site | |
| | sfGFP; CamR | |
| C104-22aa | JM109 pSB1C3-phaC_fusion-phaA-phaB [C104]; | This study |
| | phaCAB operon under the control of the J23104 | |
| | promoter; PhaC fusion: PhaC, 22aa Linker, TEV site | |
| | sfGFP; CamR | |
| C104-112aa | JM109 pSB1C3-phaC_fusion-phaA-phaB [C104]; | This study |
| | phaCAB operon under the control of the J23104 | |
| | promoter; PhaC fusion: PhaC, 112aa Linker, TEV site | |
| | sfGFP; CamR | |
| [1] PBD- | JM109 pSB103-C104; Secondary module with J23104 | This study |
| AffiEGFR1907 | promoter, B0034 RBS, PhaR derived PHA binding | |
| | domain (PBD), 112aa Linker, TEV site, sfGFP, 12aa | |
| | Linker and EGFR binding affibody-1907; CamR | |
| [2] PBD- | JM109 pSB1C3-C104; Secondary module with J23104 | This study |
| AffiZHER2-342 | promoter, B0034 RBS, PhaR derived PHA binding | |
| | domain (PBD), 112aa Linker, TEV site, sfGFP, 12aa | |
| | Linker, and HER2 binding affibody342; CamR | |
| [3] PBD-HEP | JM109 pSB1C3-C104; Secondary module with J23104 | This study |
| | promoter, B0034 RBS, PhaR derived PHA binding | |
| | domain (PBD), 112aa Linker, TEV site, sfGFP, 12aa | |
| | Linker and Heparin binding peptide; CamR | |
| [4] HspA- | JM109 pSB1C3-C104; Secondary module with J23104 | This study |
| AffiEGFR1907 | promoter, B0034 RBS, Escherichia coli heat shock | |
| | protein A, 112aa Linker, TEV site, sfGFP, 12aa Linker | |
| | and EGFR binding affibody-1907; CamR | |
| [5] HspA- | JM109 pSB1C3-C104; Secondary module with J23104 | This study |
| AffiZHER2-342 | promoter, B0034 RBS, Escherichia coli heat shock | |
| | protein A, 112aa Linker, TEV site, sfGFP, 12aa Linker | |
| | and HER2 binding affibody-342; CamR | |
| [6] HspA-HEP | JM109 pSB1C3-C104; Secondary module with J23104 | This study |
| | promoter, B0034 RBS, Escherichia coli heat shock | |
| | protein A, 112aa Linker, TEV site, sfGFP, 12aa Linker | |
| | and Heparin Binding peptide; CamR | |
| [7] PBD-HIS- | JM109 pSB1C3-C104; Secondary module with J23101 | This study |
| AffiEGFR1907 | promoter, B0034 RBS, PhaR derived PHA binding | |
| | domain (PBD), 112aa Linker, TEVsite, 4aa Linker, | |
| | 6xHIS tag, 5aa Linker and EGFR binding affibody-1907. | |
| [8] PBD- | JM109 pSB1C3-C104; Secondary module with J23101 | This study |
| AffiEGFR1907-HIS | promoter, B0034 RBS, PhaR derived PHA binding | |
| | domain (PBD), 112aa Linker, TEVsite, 9aa Linker, | |
| | EGFR binding affibody-1907 and 6xHIS tag. | |

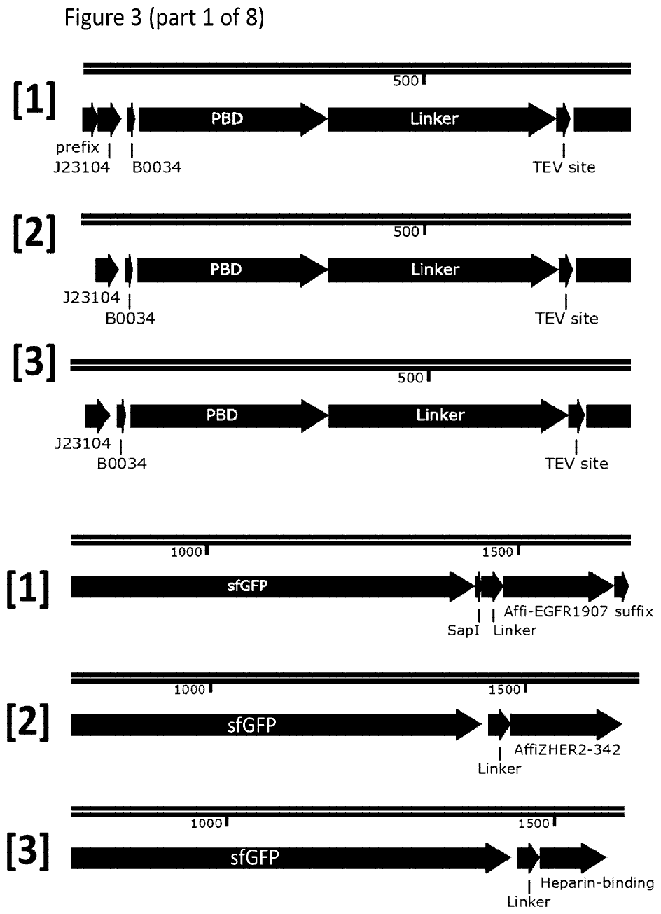

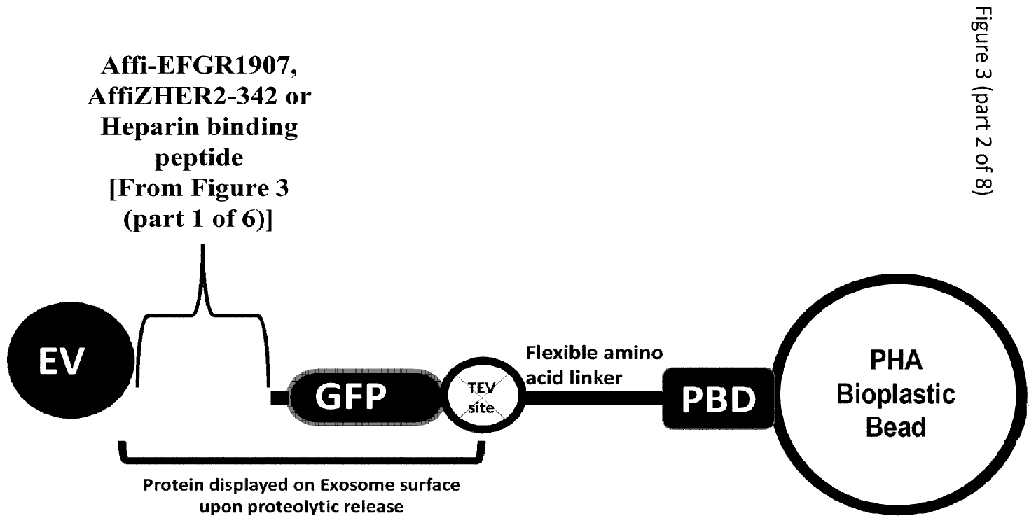

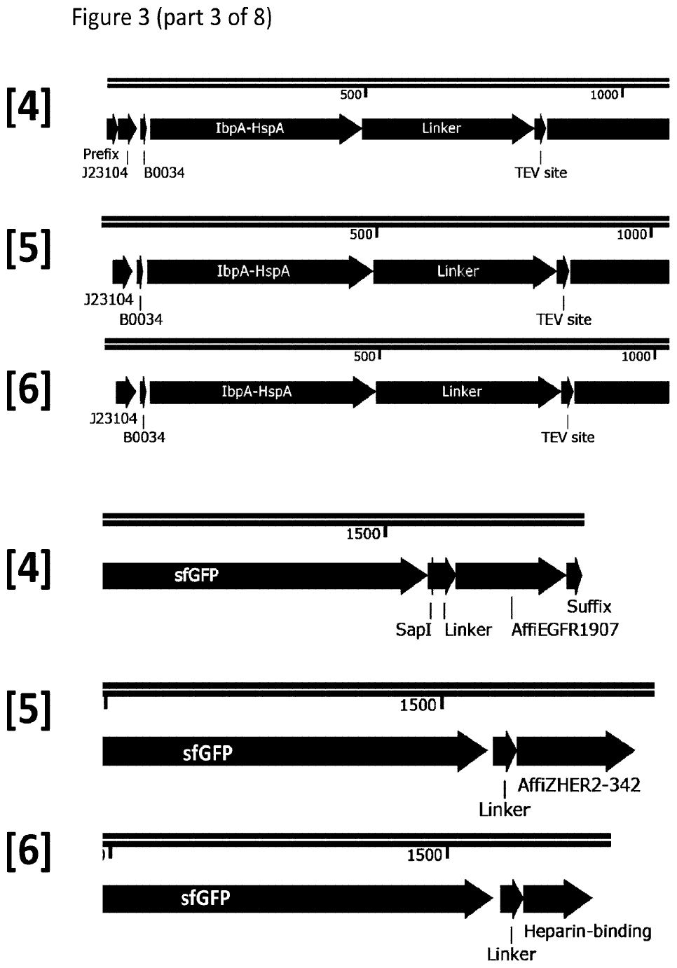

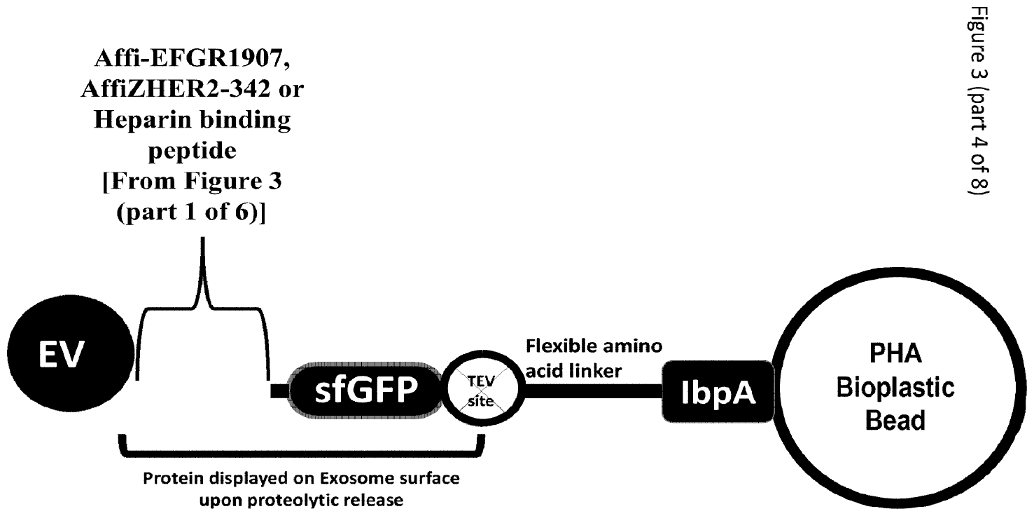

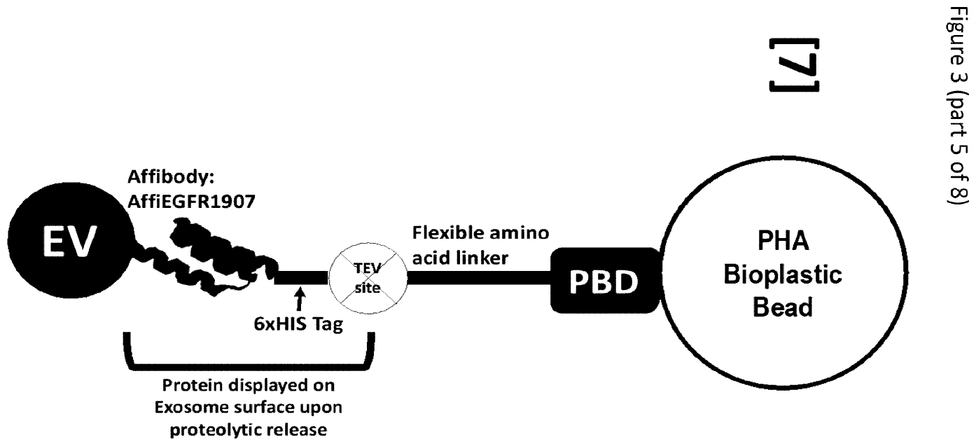

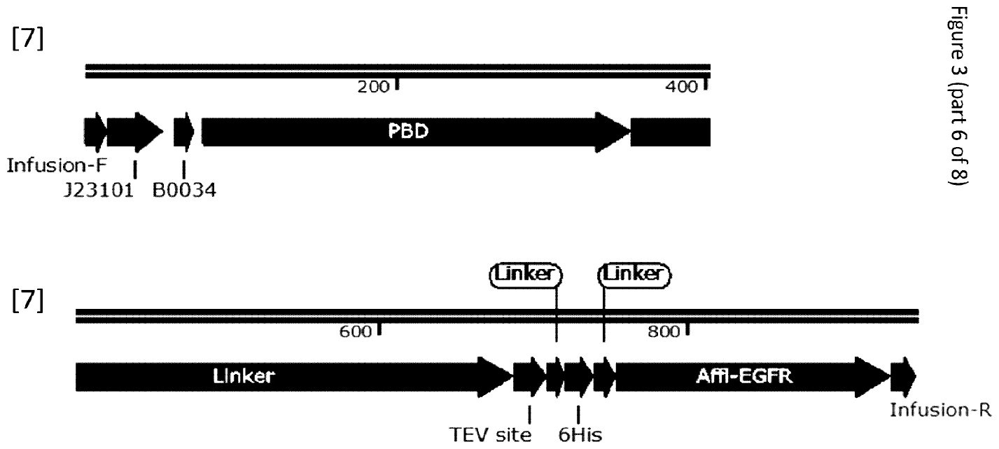

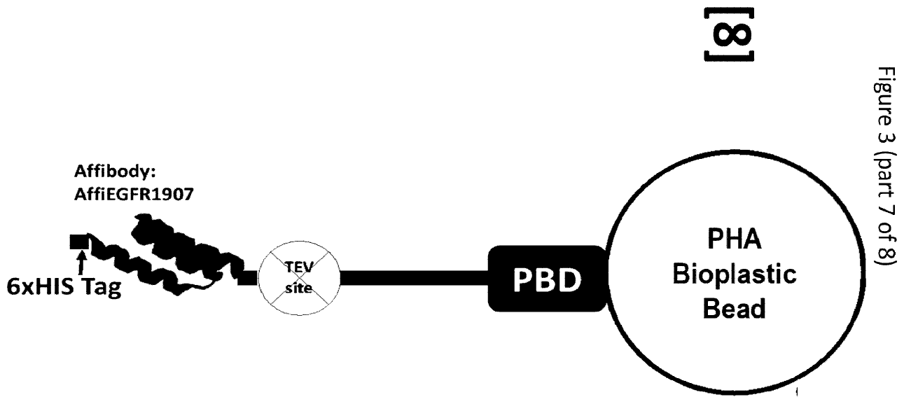

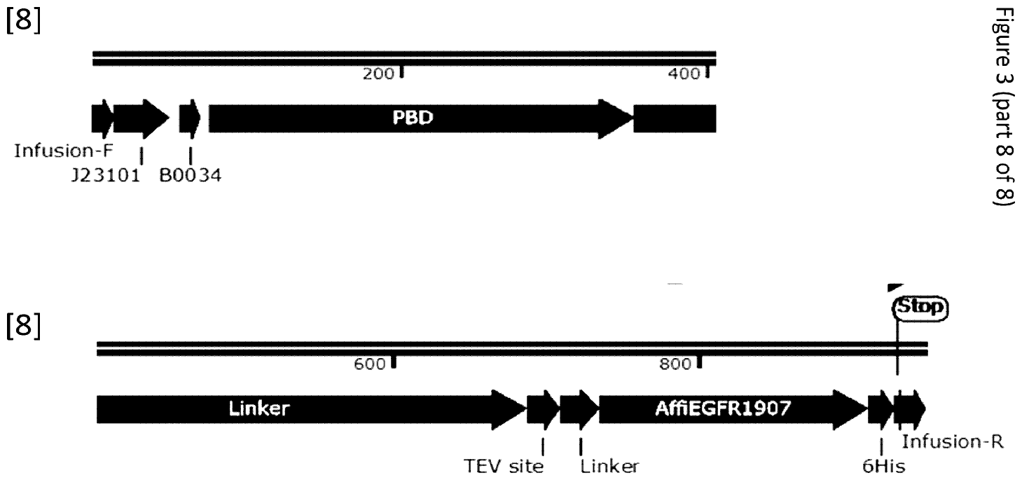

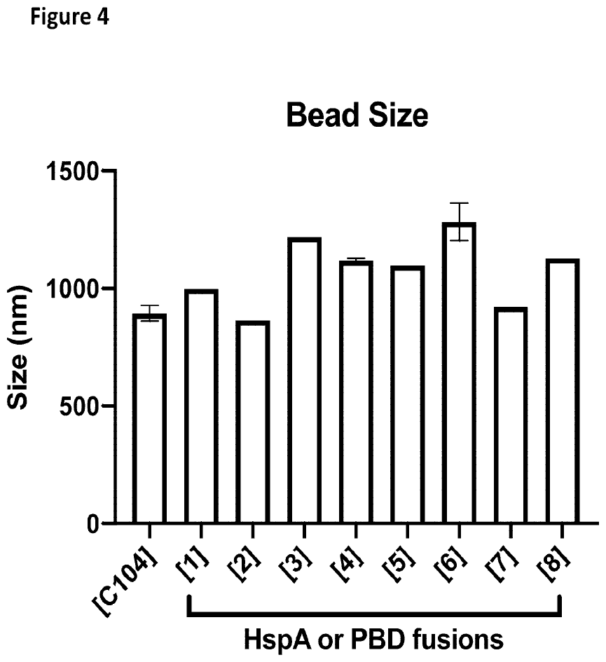

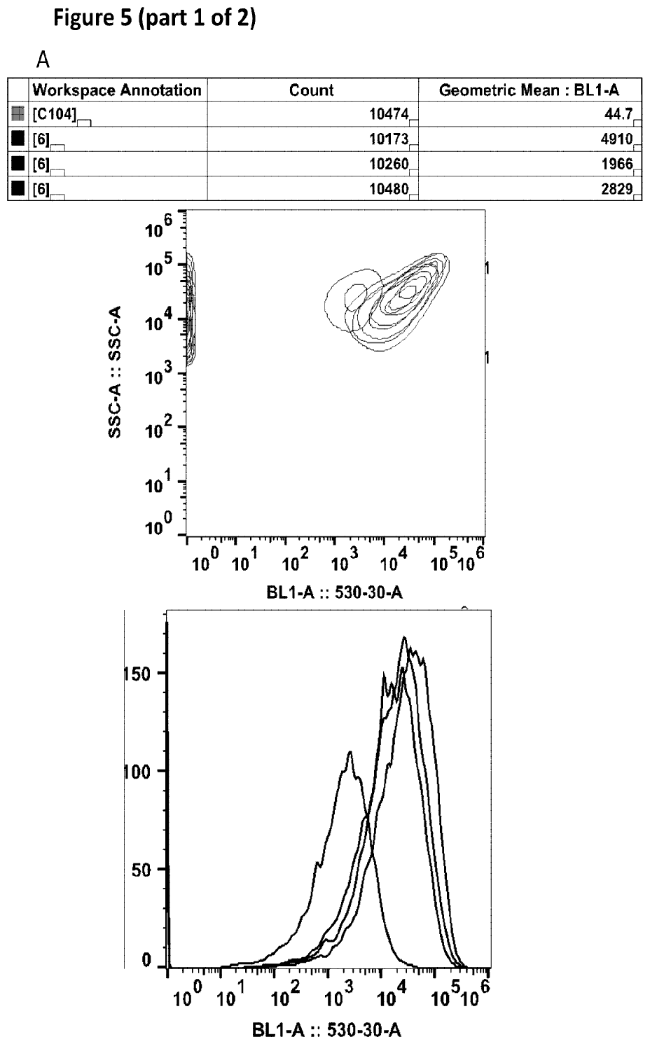

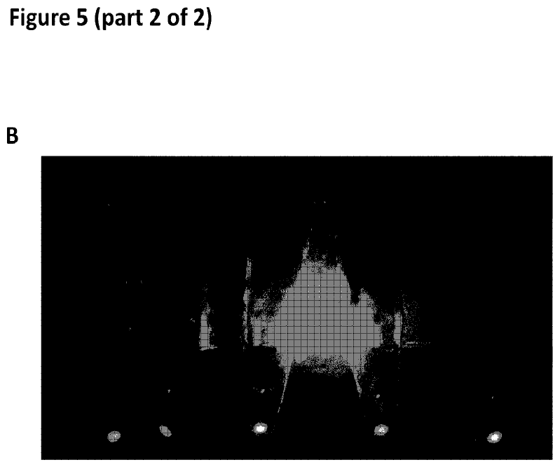

In total eight extracellular vesicle-capture fusion protein constructs were designed as IDT gblocks and then cloned into C104 vector as a separate expression module with their own regulatory elements (promoter and RBS). These constructs are termed [1] PBD-AffiEGFR1907, [2] PBD-AffiZHER2-342, [3] PBD-HEP, [4] HspA-AffiEGFR1907, [5] HspA-AffiZHER2-342, [6] HspA-HEP, [7] PBD-HIS-AffiEGFR1907 and [8] PBD-AffiEGFR1907-HIS. Once these strains were verified by sequencing PHAs production cultures were setup and PHAs particles were isolated, as described in materials and methods. Dynamic Light Scattering (DLS) analysis of isolated particles revealed that these PHAs particles were typically ˜1 μm in size (FIG. 3). Visual and Flow cytometry analysis of HspA-HEP [6] particles revealed that, as designed, their surfaces were fluorescently labelled with sfGFP (FIG. 4). Functionalised PHAs particles from constructs 1-5 weren't particularly fluorescent in comparison to control, non-functionalised beads (C104) which we hypothesise relates to likely misfolding of sfGFP within the complex fusion protein designs that we engineered. Therefore constructs 7 and 8 were designed without sfGFP, but instead incorporate HIS-tags that enable labeling with an Anti-HIS antibody. As expected, flow cytometry analysis of Anti-HIS-PE conjugated antibody labelling of constructs 7 and 8 revealed that they are bound to the surface of their respective PHAs beads (FIG. 5).

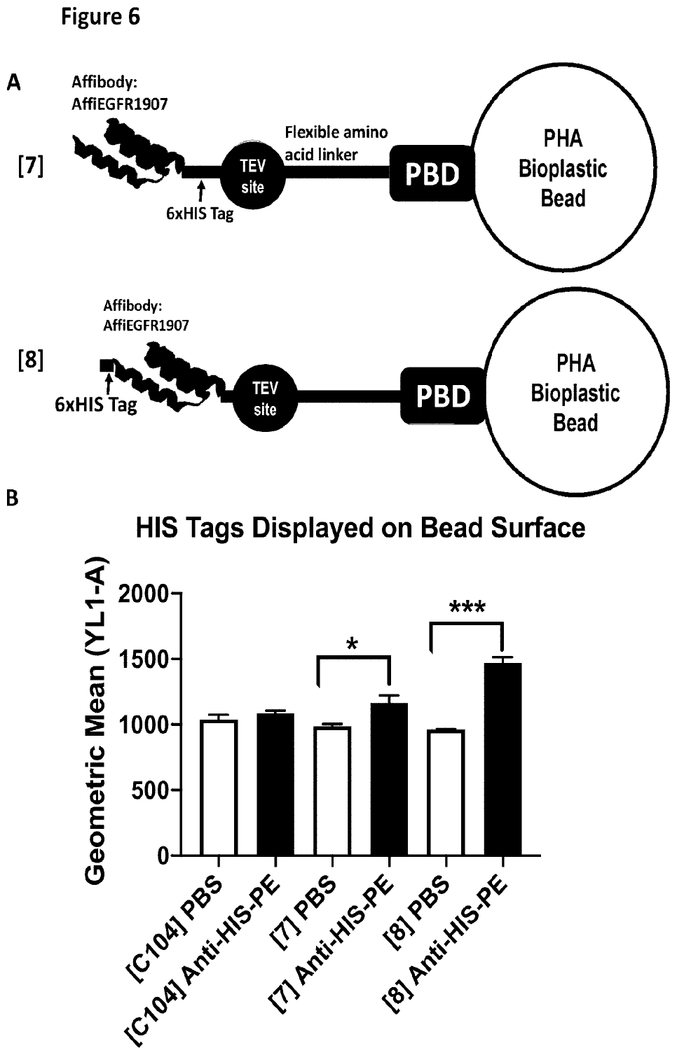

Functionalised PHAs particles from constructs 1-7 were screened in an extracellular vesicle binding assay (see materials and methods). Essentially, these extracellular vesicle-capture particles were incubated with HEK293 cell conditioned media and then stained with either a PE-conjugated control antibody or a cocktail of antibodies targeting EV surface markers (CD9, CD63, CD81). Flow cytometry analysis of these antibody stained, extracellular vesicle capture particles revealed that several constructs: [1], [4], [6] and [7] stained positive for EV markers, indicating EV capture (FIG. 6).

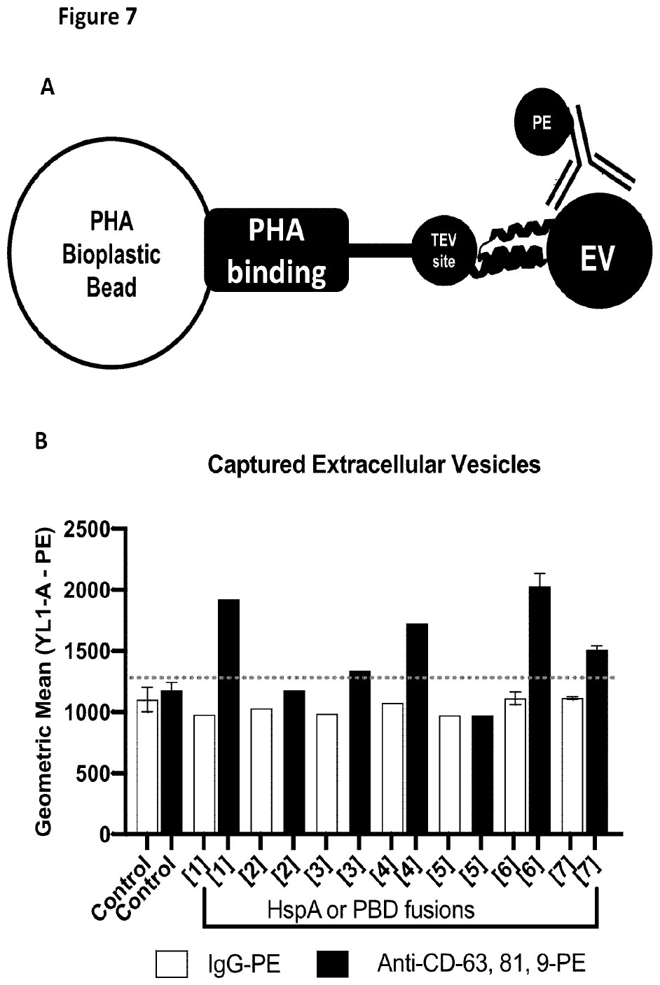

Additionally, we demonstrated that TEV treatment removes sfGFP from the PHAs particle surface, as indicated visually by pelleted PHAs particles (FIG. 7). Thus, these constructs enable extracellular vesicle release from the PHAs-particle surface.

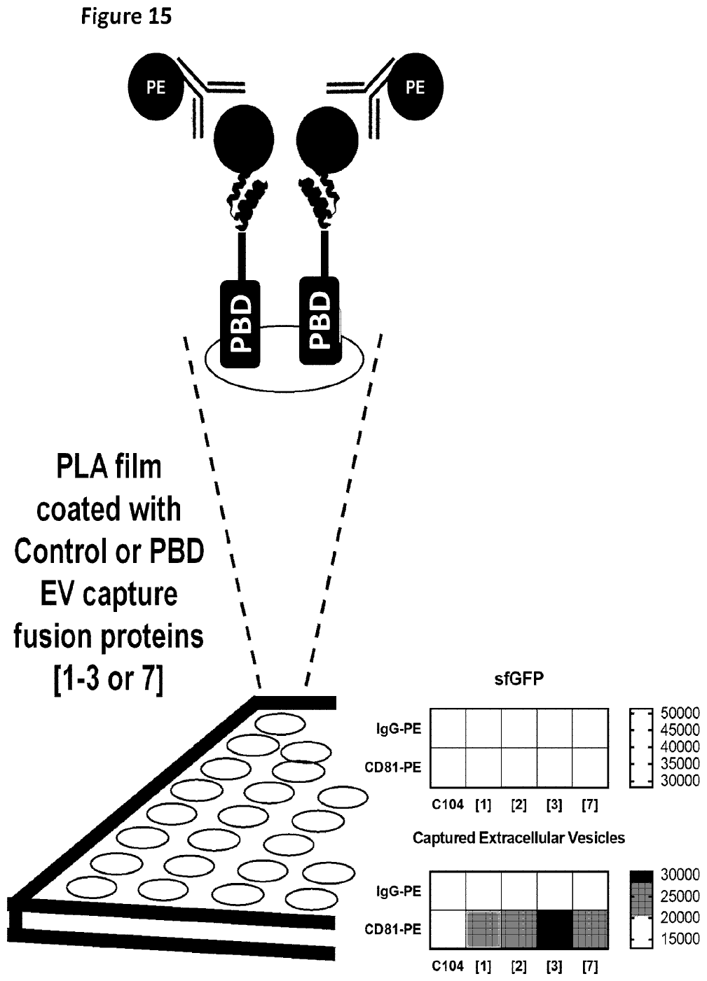

Furthermore, we have also demonstrated that PBD-based EV capture constructs ([1-3] and [7]) can also bind onto poly lactic acid (PLA) films and then be used to immobilise EVs (FIG. 15). Thus, these data demonstrate the flexibility and utility of our engineered fusion protein designs.

### Example 2—Materials and Methods

Bacterial Strains and General Growth Conditions

The constructs and strains used in this study are listed in Table 1. Escherichia coli JM109 was used for both cloning and production of exosome capture beads. For plasmid recovery E. coli strains were grown in Luria-Bertani (LB) media supplemented with 34 μg/ml Chloramphenicol (final concentration) and cultured at 37° C. with shaking (220 rpm). During PHAs bead production E. coli strains were grown in Terrific-Broth (TB) supplemented with 34 μg/ml Chloramphenicol (final concentration) and 3% glucose (w/v), cultured at 37° C. with shaking (220 rpm).

Construct Assembly

E. coli JM109 pSB1C3 EV104 (EV104) is an empty vector control plasmid that has been used previously (Kelwick et al., 2015; 2018). E. coli JM109 pSB1C3 C104 (C104-(BBa_K1149052) strain harbours a phaCAB-operon under the control of a strong constitutive promoter (J23104) and an engineered RBS (B0034) that is used to generate non-functionalised PHAs particles (Kelwick et al., 2015; 2018). Constructs C104-Vn96, C104-MT1-Af7p, C104-12aa, C104-22aa, C104-112aa, [1] PBD-AffiEGFR1907, [2] PBD-AffiZHER2-342, [3] PBD-HEP, [4] HspA-AffiEGFR1907, [5] HspA-AffiZHER2-342, [6] HspA-HEP, [7] PBD-HIS-AffiEGFR1907 and [8] PBD-AffiEGFR1907-HIS were designed as, codon optimised, IDT gBlocks and then cloned using In-fusion (Takara/Clontech) into C104 vector.

Vn96, peptide targeting heat shock proteins sequence sourced from:
Ghosh, A., Davey, M., Chute, I. C., Griffiths, S. G., Lewis, S., Chacko, S., et al. (2014). Rapid Isolation of Extracellular Vesicles from Cell Culture and Biological Fluids Using a Synthetic Peptide with Specific Affinity for Heat Shock Proteins. PLoS One 9, e110443. doi:10.1371/journal.pone.0110443.

MT1-Af7p, peptide targeting MMP14 sequence sourced from:
Zhu, L., Wang, H., Wang, L., Wang, Y., Jiang, K., Li, C., et al. (2011). High-affinity peptide against MT1-MMP for in vivo tumor imaging. J. Control. Release 150, 248-255. doi:10.1016/j.jconrel.2011.01.032.

AffiEGFR1907, EGFR targeting Affibody sequence sourced from:
Friedman, M., Orlova, A., Johansson, E., Eriksson, T. L. J., Höidén-Guthenberg, I., Tolmachev, V., Nilsson, F. Y., Ståhl, S., 2008. Directed Evolution to Low Nanomolar Affinity of a Tumor-Targeting Epidermal Growth Factor Receptor-Binding Affibody Molecule. J. Mol. Biol. 376, 1388-1402. doi:10.1016/j.jmb.2007.12.060

AffiZHER2-342, HER2 targeting affibody sourced from:
Eigenbrot, C., Ultsch, M., Dubnovitsky, A., Abrahmsen, L., Hard, T., 2010. Structural basis for high-affinity HER2 receptor binding by an engineered protein. Proc. Natl. Acad. Sci. 107, 15039-15044. doi:10.1073/pnas.1005025107

Heparin binding peptide sequence sourced from:
Morris, J., Jayanthi, S., Langston, R., Daily, A., Kight, A., McNabb, D. S., Henry, R., Kumar, T. K. S., 2016. Heparin-binding peptide as a novel affinity tag for purification of recombinant proteins. Protein Expr. Purif. 126, 93-103. doi:10.1016/j.pep.2016.05.013

The DNA sequences of all cloned inserts/constructs were verified using the sequencing service provided by Eurofins Genomics GmbH (Ebersberg, Germany), which typically provided sequencing reads of >800 bp. Sequencing chromatograms were analysed using SnapGene software (v4.1), to ensure quality, and sequencing results were aligned using Serial Cloner (v2-6-1) alignment tool against reference sequences in order to confirm that there were no discrepancies between cloned and reference sequences. Inserts were fully sequenced.

Production and Purification of Exosome Capture Beads

Briefly, glycerol stocks of E. coli JM109 strains engineered with either a negative control plasmid (EV104), a phaCAB-operon (C104) or an extracellular vesicle-capture construct ([1-8]) were used to inoculate flasks containing Terrific Broth supplemented with 3% (w/v) glucose and 34 μg/ml chloramphenicol (Cam) and then these were cultured at 37° C. for 24 h, with shaking at 200 rpm.

Post PHAs production, these strains were pelleted using centrifugation (4000 rpm, Eppendorf) and washed three times with PBS (1×). Cell pellets were re-suspended in 1 ml PBS per gram of cell pellet and sonicated. Samples were sonicated on ice, using a Vibra-cell VCX130 sonicator (SONICS, Newtown, USA) with 6 mm diameter probe. Sonication settings were (3×40 s with 1-min cooling interval; output frequency: 20 kHz; amplitude: 50%). Post-lysis samples were centrifuged (6000×g) and re-suspended with vortexing (5 seconds). The samples were then sonicated a second time on ice. Sonication settings were 2×20 s with 1-min cooling interval: Output frequency: 20 KHz, Amplitude: 50%. Post-lysis samples were centrifuged (6000×g) and the supernatant was discarded, then washed twice with PBS. Finally, the particles were re-suspended as 20% slurry (pellet w/v PBS) with 2 μl kanamycin (25 ug/ml).

DLS

PHAs bead size was analysed using a Malvern Zetasizer Nano ZS (Malvern Instruments, Malvern, UK) DLS system. Harvested PHAs beads were diluted 100-fold in PBS (1×) prior to DLS analysis. At least three replicates were measured per sample at 25° C.

Flow Cytometry

PHAs bead fluorescence and PE antibody detection was measured using an Attune NxT Flow Cytometer (Thermo Fisher Scientific, MA, USA). ˜10,000 events per sample were measured (GFP: BL1-A detector, Excitation 488 nm/Emission 530/30 nm, perCP Cy5.5 Excitation 488 nm/Emission 695/40 nm, Cell Mask Orange/: YL1-A Excitation 561 nm/Emission 585-16 nm or PE: YL1-A, Excitation 561 nm/Emission 578 nm). At least three replicates for each sample were used. Data analysis was performed using FlowJo (vX 10.4.1) software. The gate strategy was based upon standard 1 μm diameter beads (Flow Cytometry Sub-Micron Size Reference Kit, Thermo Fisher Scientific, MA, USA).

Exosome Capture

HEK293F cells (Thermo #R79007) were cultured in Freestyle expression medium (Thermo #12338001) within 1L suspension flasks at 37° C. with 8% CO₂ and shaking at 110 rpm for 48 h. At which time cell density had reached 2.61×10⁷ cells/ml. Conditioned media was harvested and then centrifuged 300 g for 10 min. The supernatant was removed and further centrifuged 2,500 g for 10 min at room temperature. Subsequently, the supernatant was filtered (0.2 μm filter) as a final step to aid in the removal of cells and large debris. 2 μl of harvest PHAs beads were incubated with 298 μl HEK conditioned media, gentle vortexed (for 5 seconds) and incubated on a carousel mixer for 60 min at room temperature.

In order to analyse captured EVs these EV/PHAs-bead mixtures were treated with either 5 μl of CD63-PerCP Cy5.5 (BD #565426), 3 μl of Mouse IgG1-PE Kappa isotype control (MACS Miltenyi biotec #130-113-200) or a cocktail consisting of 1 μl each of the following EV marker antibodies: PE-CD9, human (MACS Miltenyi biotec #130-103-955), PE-CD63, human (MACS Miltenyi biotec #130-100-153) and PE-CD81, human (MACS Miltenyi biotec #130-118-342). Samples were gently vortexed (for 5 seconds) and incubated for 30 minutes at room temperature. Post-incubation the samples were centrifuged (6000 g) for 10 min and the supernatant was removed. The beads were washed with 1 ml PBS (1×) and then analysed on an Attune flow cytometer, as described above.

In Detail:

Initially, PHAs EV capture beads were washed with DPBS (1×) and then pelleted using centrifugation—14,000×g for 5 minutes. Post-centrifugation, the DPS supernatant was removed and the pelleted EV capture beads were re-suspended in 2 ml of cell conditioned media (the media of the EV producing cell line). EV capture beads were incubated in cell conditioned media on a rotating carousel for 1 hour at room temperature. Post-incubation, PHA captured EVs were pelleted using centrifugation (14,000×g for 5 minutes) and the cell conditioned media was removed. As before, the PHAs beads were subsequently washed with PBS. Captured EVs were released from PHAs beads using AcTEV protease. Briefly, post-washing, PHA capture beads were re-suspended in 100 ul TEV assay reactions—1 μl AcTEV (10U; Life Technologies, CA, USA), 1 μl of dithiothreitol (DTT), 5 μl of TEV reaction buffer (20×; 1M Trix-HCl pH8.0, 10 mM EDTA), 93 μl of PBS (1×) and then incubated at 30° C. with shaking at 500 rpm (Eppendorf Thermo Mixer C) for 2 hours. PHAs beads were subsequently separated from PHAs beads using centrifugation (14,000×g for 5 minutes) or gravity sedimentation. EV containing supernatants were stored at −80° C. for downstream processing or analysis.

High-Throughput EV-Capture Assay

Control (C104) and PHAs-based EV capture strains (C104-Vn96, C104-MT1-Af7p) were inoculated, from glycerol stocks, into wells on a 96-well plate containing 100 μl Terrific Broth supplemented with 3% (w/v) glucose and 34 μg/ml chloramphenicol (Cam) and then these were cultured at 37° C. for 24 h, with shaking at 200 rpm. PHAs beads were isolated from these strains using a plate sonicator (QSonica #Q800R3; Sonication settings were 3×40 s with 1-min cooling interval and Programme 4). Post-sonication, the 96-well plates were centrifuged (2250 g for 10 min at 4° C.). Cell lysate supernatants were removed, and isolated PHAs-beads were washed 3× with 100 μl PBS (1×). During each wash PHAs-based were re-pelleted using centrifugation (2250 g for 10 min at 4° C.). Pre-stained (Cell Mask Orange) HEK293 EVs were mixed within 100 μl PBS (1×) and added to appropriate wells for EV binding. Control samples (e.g. EVs or PBS only and flow cytometry calibration beads) were also setup in appropriate wells. Plates were subsequently incubated at room temperature with shaking (200 rpm) and then analysed using flow cytometry as described above. Bead samples were also pipetted into a fresh 96-well plate and stained with Nile Red (0.5 μg/ml final concentration) and analysed for PHAs content on a CLARIOstar plate reader (BMG, Labtech, Ex. 560-15 nm/Em. 610-20).

EV Capture on Functionalised Poly Lactic Acid Film

C104 and PBD-based construct strains [1-3 and 7] were cultured overnight in 6 ml Terrific Broth supplemented with 34 μg/ml chloramphenicol (Cam) at 37° C. for 24 h, with shaking at 220 rpm. Glucose was excluded from these cultures to minimise PHAs bead production and to ensure free fusion proteins were produced. Post-culture, these overnights were centrifuged (2200 g for 10 minutes at 4° C.) to form cell pellets. Once pelleted, media supernatant was removed, and the cell pellets were washed with 5 ml PBS (1×). Cell pellets were then lysed within 300 μl Bugbuster (Merck/Sigma Aldrich, #70923-3) and incubated at room temperature for 10 min. Post-lysis, cell lysates were transferred to 2 ml tubes and centrifuged (14000 g for 10 min at room temperature) and the resultant lysate-supernatants were stored on ice. Poly lactic acid (PLA) film (Sigma, Aldrich #GF27769304) was cut into several discs (0.6 cm in diameter, 0.05 mm thickness) and placed into appropriate wells in a 96-well plate. These PLA film discs were washed with 100 μl PBS (1×) and then blocked with 100 μl PBS with 5% BSA (w/v) for 10 minutes at room temperature with shaking (Setting 85 Stuart SSM1 mini orbital shaker). Post-incubation, blocking solution was removed and PLA film discs were washed with 200 μl PBS (1×). 50 μl of appropriate cell lysates (see above) were applied to PLA film discs within appropriate plate wells and were incubated, to facilitate PBD-fusion protein binding, for 30 minutes at room temperature with shaking (Setting 85 Stuart SSM1 mini orbital shaker). Then 150 μl PBS (1×) was added to each well and the samples were incubated for a further 30 minutes at room temperature with shaking (Setting 85 Stuart SSM1 mini orbital shaker). Post-incubation, these PBS/cell lysate solutions were removed and 50 μl A549 EV solutions (20 μg total protein of A549 EVs reconstituted into 50 μl PBS [1×]; HansaBioMed/Newmarket scientific, #HBM-A549-100) were pipetted onto PLA discs in appropriate wells. These samples were subsequently incubated, to facilitate EV-binding to PLA-film bound PBD-fusion proteins, for 30 minutes at room temperature with shaking (Setting 85 Stuart SSM1 mini orbital shaker). 50 μl of antibody solutions (PBS [1×] with either 1 μl of IgG-PE (MACS Miltenyi biotec #130-113-200) or 1 μl of CD81-PE ((MACS Miltenyi biotec #130-118-342) were pipetted onto PLA discs in appropriate wells. Samples were subsequently incubated for 30 minutes at room temperature with shaking (Setting 85 Stuart SSM1 mini orbital shaker). Post-incubation, 100 μl PBS [1×]) were pipetted onto PLA discs in appropriate wells and these samples were incubated for an additional 10 minutes at room temperature with shaking (Setting 85 Stuart SSM1 mini orbital shaker). Post-incubation PLA discs were washed 3× with 200 μl PBS [1×]). EV-capture PLA discs were subsequently measured (whole-well scanning and averaging) on a CLARIOstar plate reader (sfGFP Ex. 483-14 nm/Em. 530-30; PE Ex. 496-15 nm/Em. 578-20).

### CONCLUSIONS

These functionalised PHAs beads and in particular the fusion protein designs represent a novel way to isolate extracellular vesicles. We envision that their modular nature are enabling us to automate the cloning and generation of libraries of exosome capture beads that incorporate many different affibody/peptide sequences. Likewise, the shift from PhaC towards a PhaR-derived binding domain (PBD) affords greater flexibility in that PhaR can bind to many other polymers beyond PHAs including poly(L-lactide) (PLLA), polyethylene (PE), and polystyrene (PS). Thus, enabling different form factors for different exosome isolation applications.

The following numbered paragraphs describe further embodiments of the invention:

1. A method for isolating extracellular vesicles from a sample, the method comprising:
(i) providing a composition of coated biopolymer particles, the biopolymer particles being coated with a fusion protein;
(ii) contacting the composition comprising the coated biopolymer particles with the sample comprising extracellular vesicles under conditions which allow the formation of a coated biopolymer particle-extracellular vesicle complex; and
(iii) isolating the coated biopolymer particle-extracellular vesicle complex.
   2. A method for functionalising the surface of extracellular vesicles, the method comprising:
(i) providing a composition of coated biopolymer particles, the biopolymer particles being coated with a fusion protein that comprises a functionalisation domain;
(ii) contacting the composition comprising the coated biopolymer particles with a sample comprising extracellular vesicles under conditions which allow the formation of a coated biopolymer particle-extracellular vesicle complex; and
(iii) processing the coated biopolymer particle-extracellular vesicle complex so as to provide a) a functionalisation domain-associated extracellular vesicle and b) a fusion protein portion-associated biopolymer particle.
   3. The method of Paragraph 1 or 2 wherein the extracellular vesicle is a microvesicle, an apoptotic body, an ectosome, an exosome, an exomere, a small oncosomes, a large oncosome or an exosome mimetic.
   4. The method of any one of Paragraphs 1-3 wherein the extracellular vesicle is an exosome.
   5. The method of any one of Paragraphs 1-4 wherein the contacting the composition comprising the coated biopolymer particles with a sample comprising extracellular vesicles, occurs in aqueous solution, at a temperature between 4° C.-60° C., at a pH between 6.0-8.5.
   6. The method of any one of Paragraphs 1-5 wherein the biopolymer particle comprises one or more of a polyhydroxyalkanoate (PHA), a poly(L-lactide) (PLLA), a polyethylene (PE), a polystyrene (PS), or a polythioester (PTE).
   7. The method of any one of Paragraphs 1-6 wherein the PHA comprises poly(3-hydroxybutyrate) (P(3HB)), poly(4-hydroxybutyrate) (P(4HB)), polyhydroxyvalerate (PHV), poly(3-hydroxyhexanoate) (P(3HHx)), poly(3-hydroxyheptanoate) (P(3HH)), poly(3-hydroxyoctanoate) (P(3HO)), poly(3-hydroxynonanoate) (P(3HN)), poly(3-hydroxydecanoate) (P(3HD)), poly(3-hydroxybutyrate-co-3-hydroxyvalerate) (PHBV), 3-hydroxybutyrate and 4-hydroxybutyrate (P3HB4HB), poly(3HB-co-3-hydroxyvalerate) (P(3HB-co-3HV)), poly(3HB-co-3-hydroxyhexanoate) (P(3HB-co-3HHx)), poly(3HB-co-3-hydroxy-4-methylvalerate) (P(3HB-co-3H4MV)), or poly(3HB-co-medium-chain-length-3HA) (P(3HB-co-mcl-3HA)).
   8. The method of any one of Paragraphs 1-7 wherein the biopolymer particle is a PHA-blended biopolymer particle comprising PHA and a further biopolymer, optionally where the further biopolymer is selected from the group consisting of a poly(lactic acid)-poly(hydroxybutyrate) (PLA-PHB), starch-PHA, poly(L-lactide) (PLLA), a polyethylene (PE), a polystyrene (PS), or a polythioester (PTE).
   9. The method of Paragraph 8 wherein the PHA-blended biopolymer comprises poly(lactic acid)-poly(hydroxybutyrate) (PLA-PHB) or starch-PHA.
   10. The method of any one of Paragraphs 1-9 wherein the coated biopolymer particles have been formed by a method comprising the steps of:
(i) providing a host cell that produces
   a) biopolymer particle; and
   b) a fusion protein capable of coating the biopolymer particles in the cells;
(ii) cultivating the host cell under conditions suitable for the production of biopolymer particles coated with the fusion protein; and
(iii) isolating the coated biopolymer particles from the host cell.
   11. The method of Paragraph 10 wherein the host cell comprises
i) A biopolymer particle production nucleic acid construct,
   optionally wherein the biopolymer particle production nucleic acid construct expresses one or more proteins that produce one or more of a polyhydroxyalkanoate (PHA), a poly(L-lactide) (PLLA), a polyethylene (PE), a polystyrene (PS), or a polythioester (PTE),
   optionally wherein the biopolymer particle production nucleic acid construct is a PHA production nucleic acid construct that expresses a phaCAB operon, optionally comprising
      a constitutive promoter,
      a synthetic ribosome binding site linked to a polynucleotide encoding a phaC gene, and/or
      natural ribosome binding sites linked to a polynucleotide encoding a phaA gene and a polynucleotide encoding a phaB gene;
         and
ii) A fusion protein production nucleic acid.
   12. The method of any one of Paragraphs 1-9 wherein the biopolymer particles have been formed by a cell-free method comprising the steps of:
(i) providing a solution comprising
   a) at least one biopolymer particle production nucleic acid construct, that comprises a first promoter, optionally a first inducible or repressible promoter suitable for the production of biopolymer particles; and
   b) at least one fusion protein production nucleic acid construct that comprises a second promoter, optionally a second inducible or repressible promoter
(ii) maintaining the solution under conditions suitable for expression of the at least one biopolymer particle production nucleic acid construct and the at least one fusion protein production nucleic acid construct, and for formation of biopolymer particles coated with the fusion protein; and optionally
(iii) isolating the coated biopolymer particles from the solution.
   13. The method of any one of Paragraphs 1-9 wherein the coated biopolymer particles have been formed by a method comprising the steps of:
(i) providing a biopolymer particle;
(ii) providing a fusion protein capable of coating the biopolymer particle; and
(iii) contacting the biopolymer particle with the fusion protein under conditions suitable for formation of biopolymer particles coated with the fusion protein.
   14. The method of Paragraph 10 or 11, wherein the host cell is: a bacterial cell, optionally a cyanobacterial cell; an archaeal cell, optionally a haloarchaeal cell; a fungal cell, optionally a yeast cell; or a plant cell.
   15. The method of Paragraph 14, wherein the bacterial cell is selected from the genera Alcaligenes, Azotobacter, Bacillus, Chlorogloea, Cupriavidus, Escherichia, Gloeothece, Haloferax, Halomonas, Lactobacillus, Pseudomonas, Ralstonia, Spirulina, Synechococcus, or Thermus.
   16. The method of Paragraph 14 or 15, wherein the bacterial cell is a cell selected from the group comprising Alcaligenes latus, Azotobacter chroococcum, Azotobacter vinelandii, Bacillus amyloliquefaciens DSM7, Bacillus laterosporus, Bacillus licheniformis, Bacillus macerans, Bacillus cereus, Bacillus circulans, Bacillus firmus G2, Bacillus subtilis I68, Bacillus subtilis K8, Bacillus sphaericus X3, Bacillus megaterium Y6, Bacillus coagulans, Bacillus brevis, Bacillus sphaericus ATCC 14577, Bacillus thuringiensis, Bacillus mycoides RLJ B-017, Bacillus sp. JMa5, Bacillus sp. INT005, Chlorogloea fritschii, Cupriavidus necator, Escherichia coli, Haloferax mediterraneis, Halomonas elongate, Halomonas species TD01, Halomonas sp. KM-1, Halomonas smyrnensis, Halomonas profundus, Pseudomonas aeruginosa, Pseudomonas mendocina PSU, Pseudomonas oleovorans, Pseudomonas putida, Ralstonia eutropha, or Thermus thermophilus.
   17. The method of Paragraph 14, wherein the yeast cell is a Saccharomyces cerevisiae or Pichia pastoris cell, the fungal cell is a Fusarium solani Thom cell, or the plant cell is an Arabidopsis thaliana, Camelina sativa, Nicotiana tabacum or Saccharum officinarum cell.
   18. The method of any one of Paragraphs 10, 11, 13-17 wherein the biopolymer particles are isolated from the host cell by disrupting the cell and isolating the particles.
   19. The method of Paragraph 18 wherein disrupting the cell is performed by physical disruption.
   20. The method of Paragraph 19 wherein the physical disruption is performed by sonication, a cell press, detergent lysis, freeze-thawing, bead-beating, hypotonic cell disruption, or enzymatic disruption.
   21. The method of any one of Paragraphs 18-20 wherein the isolating the particles is performed using a cell sorter, centrifugation, gravity sedimentation, electrophoresis, filtration, size exclusion chromatography or affinity chromatography.
   22. The method of any one of Paragraphs 12, or 18-21 wherein the isolating the particles is performed using filtration.
   23. The method of any one of Paragraphs 1-22 wherein the mean diameter of the uncoated biopolymer particle is:
   a) between 50 nm and 1,500 nm, for example between 60 nm and 1,250 nm, 80 nm and 1,000 nm, 100 nm and 800 nm, 150 nm and 600 nm, 200 nm and 500 nm, 300 and 400 nm;
   b) less than 1,500 nm, 1,250 nm, 1,000 nm, 800 nm, 600 nm, 500 nm, 400 nm, 300 nm, 200 nm, 150 nm, 100 nm, 80 nm, 60 nm, or less than 50 nm; and/or
   c) greater than 1,500 nm, 1,250 nm, 1,000 nm, 800 nm, 600 nm, 500 nm, 400 nm, 300 nm, 200 nm, 150 nm, 100 nm, 80 nm, 60 nm, or greater than 50 nm.
   24. The method of any one of Paragraphs 1-23 wherein the mean diameter of the isolating uncoated biopolymer particle is between 500 nm and 1,500 nm.
   25. The method of Paragraph 23 or 24 wherein biopolymer particle size is determined using dynamic light scattering or flow cytometry.
   26. The method of any one of the Paragraphs 1-25 wherein:
a) between about 5% and 60% of the surface of the biopolymer particle is coated with the fusion protein, for example between 10% and 50%, 20% and 40%, for example 20% or 30% of the surface is coated with the fusion protein;
b) at least 5%, 10%, 20%, 30%, 40%, 50% or at least 60% of the surface is coated with the fusion protein; and/or
c) less than 60%, 50%, 40%, 30%, 20%, 10% or less than 5% of the surface is coated with the fusion protein.
   27. The method of any one of Paragraphs 1-26 wherein about 20% of the surface of the biopolymer particle is coated with the fusion protein.
   28. The method of Paragraph 26 or 27 wherein the percentage of the biopolymer particles that is coated with the fusion protein is determined by transmission electron microscopy or proteolytic cleavage of the fusion protein followed by protein quantification.
   29. The method of any one of Paragraphs 10, 11 or 14-28 wherein the host cell comprises:
a) between about 5 and about 60 coated biopolymer particles, for example between about 10 and about 50, about 20 and about 40, about 30;
b) at least about 5 coated biopolymer particles, at least about 10, 20, 30, 40, 50 or at least about 60; and/or
c) less than about 60 coated biopolymer particles, less than about 50, 40, 30, 20, or less than about 5.
   30. The method of Paragraph 10, 11 or 14-29 wherein the host cell comprises at least biopolymer particles, preferably wherein the host cell contains 32 biopolymer particles.
   31. The method of any one of Paragraphs 1-30 wherein the fusion protein comprises:
i) a biopolymer particle binding domain, optionally wherein the biopolymer binding domain comprises a domain capable of binding to one or more of a polyhydroxyalkanoate (PHA), a poly(L-lactide) (PLLA), a polyethylene (PE), a polystyrene (PS), or a polythioester (PTE); optionally comprises
a) PhaR-derived binding domain (PBD), optionally comprises or consists of SEQ ID NO: 2 or SEQ ID NO 1 or a sequence that has at least 70%, 75%, 80%, 85%, 90%, 91%, 92%, 93%, 94%, 95%, 96%, 97%, 98%, 99%, or 100% sequence identity to SEQ ID NO: 1 or SEQ ID NO: 2;
b) a phasin, optionally a PhaR, a PhaP, a PhaQ, a PhaF, a PhaI, or an inactive PhaZ1;
c) IbpA (HspA); or
d) PhaC.
and
ii) an extracellular vesicle binding domain, optionally wherein the extracellular vesicle binding domain is selected from a protein, a protein fragment, a binding domain, a target-binding domain, a binding protein, a binding protein fragment, an affibody, an antibody, an antibody fragment, an antibody heavy chain, an antibody light chain, a single chain antibody, a single-domain antibody, a Fab antibody fragment, an Fc antibody fragment, an Fv antibody fragment, a F(ab′)2 antibody fragment, a Fab′ antibody fragment, a single-chain Fv (scFv) antibody fragment, a camelid antibody, an IgNAR Shark antibody, a DARPin, a nanobody, an antibody binding domain, an antigen, an antigenic determinant, an epitope, a hapten, an immunogen, an immunogen fragment, biotin, a biotin derivative, an avidin, a streptavidin, a substrate, an enzyme, an abzyme, a co-factor, a receptor, a receptor fragment, a receptor subunit, a receptor subunit fragment, a ligand, an inhibitor, a hormone, a lectin, a polyhistidine, a coupling domain, a DNA binding domain, a FLAG epitope, a cysteine residue, a library peptide, a reporter peptide, and an affinity purification peptide, or a combination thereof;
optionally wherein the fusion protein further comprises:
iii) a functionalisation domain, optionally wherein the functionalisation domain is a membrane disrupting peptide or a cell targeting peptide; and/or
iv) a sequence capable of being cleaved by a protease, optionally a site specific protease, optionally a TEV protease.
   32. The method of Paragraph 31 wherein the extracellular vesicle binding domain is capable of binding specifically to an extracellular vesicle-specific surface antigen.
   33. A fusion protein comprising:
i) a biopolymer particle binding domain; and
ii) an extracellular vesicle binding domain, optionally wherein the extracellular vesicle binding domain is capable of binding specifically to an extracellular vesicle-specific surface antigen;
   optionally wherein the fusion protein further comprises:
a functionalisation domain, optionally wherein the functionalisation domain is a membrane disrupting peptide or a cell targeting peptide; and/or
a sequence capable of being cleaved by a protease, optionally a site specific protease, optionally a TEV protease.
   34. The method of any one of Paragraphs 31 or 32 or the fusion protein of Paragraph 33, wherein the biopolymer binding domain comprises a domain capable of binding to one or more of a polyhydroxyalkanoate (PHA), a poly(L-lactide) (PLLA), a polyethylene (PE), a polystyrene (PS), or a polythioester (PTE).
   35. The method or the fusion protein of Paragraph 34, wherein the biopolymer binding domain is:
a) PhaR-derived binding domain (PBD), optionally comprises or consists of SEQ ID NO: 2 or SEQ ID NO 1 or a sequence that has at least 70%, 75%, 80%, 85%, 90%, 91%, 92%, 93%, 94%, 95%, 96%, 97%, 98%, 99%, or 100% sequence identity to SEQ ID NO: 1 or SEQ ID NO: 2;
b) a phasin, optionally a PhaR, a PhaP, a PhaQ, a PhaF, a PhaI, or an inactive PhaZ1;
c) IbpA (HspA); or
d) PhaC.
   36. The method of any one of Paragraphs 31 or 32, 34 or 35, or the fusion protein of any one of Paragraphs 34-36, wherein the biopolymer particle binding domain is located at the N-terminus of the fusion protein and the extracellular vesicle binding domain is located at the C-terminus of the fusion protein.
   37. The method of any one of Paragraphs 31 or 32, 34 or 35, or the fusion protein of any one of Paragraphs 34-36, wherein the extracellular vesicle binding domain is located at the N-terminus of the fusion protein and the biopolymer particle binding domain is located at the C-terminus of the fusion protein.
   38. The method of any one of Paragraphs 31 or 32 or 34-37, or the fusion protein of any one of Paragraphs 34-37 wherein the biopolymer particle binding domain is fused to the extracellular vesicle binding domain via a linker peptide,
optionally where the fusion protein comprises a functionalisation domain the biopolymer particle binding domain is fused to the functionalisation domain via a linker peptide.
   39. The method or the fusion protein of Paragraph 38 wherein the linker peptide is between around 12-112 amino acid residues in length and comprises small, non-polar and/or small, polar amino acids.
   40. The method of any of paragraphs 31 or 32, or 34-39 or the fusion protein of any of paragraphs 34-39 wherein the fusion protein comprises a cleavable site, optionally cleavable by the TEV protease.
   41. The method or the fusion protein of Paragraph 40 wherein the cleavable site is located in the linker peptide.
   42. The method of any one of Paragraphs 31 and 32 or 34-41, or the fusion protein of any one of Paragraphs 34-41, wherein the biopolymer particle binding domain comprises a PHA binding domain of a phasin repressor protein, PhaR.
   43. The method or the fusion protein of Paragraph 42 wherein the PHA binding domain of a phasin repressor protein, PhaR, lacks DNA-binding activity.
   44. The method of any one of Paragraphs 31 or 32 or 34-43 the fusion protein of any one of Paragraphs 34-43, wherein the extracellular vesicle binding domain is selected from a protein, a protein fragment, a binding domain, a target-binding domain, a binding protein, a binding protein fragment, an affibody, an antibody, an antibody fragment, an antibody heavy chain, an antibody light chain, a single chain antibody, a single-domain antibody, a Fab antibody fragment, an Fc antibody fragment, an Fv antibody fragment, a F(ab′)2 antibody fragment, a Fab′ antibody fragment, a single-chain Fv (scFv) antibody fragment, a camelid antibody, an IgNAR Shark antibody, a DARPin, a nanobody, an antibody binding domain, an antigen, an antigenic determinant, an epitope, a hapten, an immunogen, an immunogen fragment, biotin, a biotin derivative, an avidin, a streptavidin, a substrate, an enzyme, an abzyme, a co-factor, a receptor, a receptor fragment, a receptor subunit, a receptor subunit fragment, a ligand, an inhibitor, a hormone, a lectin, a polyhistidine, a coupling domain, a DNA binding domain, a FLAG epitope, a cysteine residue, a library peptide, a reporter peptide, and an affinity purification peptide, or a combination thereof.
   45. The method of any one of Paragraphs 31 or 32 or 34-44, or the fusion protein of any one of Paragraphs 34-44 wherein the extracellular vesicle binding domain is an affibody.
   46. A nucleic acid encoding the fusion protein of any one of paragraphs 34-45.
   47. A nucleic acid construct comprising:
i) a nucleic acid encoding a fusion protein as defined in any one of Paragraphs 34-45;
ii) a nucleic acid encoding a further entity that is capable of binding to an extracellular vesicle-specific surface antigen; and
optionally a nucleic acid encoding a biopolymer synthase
operably linked to at least one promoter.
   48. The nucleic acid of Paragraph 46 or 47 wherein the promoter is an inducible promoter, a synthetic promoter, a viral promoter or a phage promoter.
   49. A biopolymer particle coated with:
i) one or more fusion proteins according to any of Paragraphs 34-45; or
ii) one or more fusion proteins as defined in any of Paragraphs 34-45 and further comprising the further entity that is capable of binding specifically to an extracellular vesicle-specific surface antigen.
   50. A host cell comprising a nucleic acid according to any of paragraphs 46-48, optionally operably linked to an inducible promoter,
wherein the host cell optionally comprises one or more nucleic acids that drive production of the biopolymer particle, optionally under the control of an inducible promoter,
optionally wherein the inducible promoter that is operably linked to the nucleic acid according to any of paragraphs 46-48 and the inducible promoter that drives production of the biopolymer particle are induced by different inducers.
   51. A kit comprising any one or more of:
i) an expression construct comprising a biopolymer production module, optionally wherein the biopolymer production module produces one or more of a polyhydroxyalkanoate (PHA), a poly(L-lactide) (PLLA), a polyethylene (PE), a polystyrene (PS), or a polythioester (PTE),
optionally wherein the biopolymer production module is a PHA production module that expresses a phaCAB operon, optionally comprising
a constitutive promoter,
a synthetic ribosome binding site linked to a polynucleotide encoding a phaC gene, and/or
natural ribosome binding sites linked to a polynucleotide encoding a phaA gene and a polynucleotide encoding a phaB gene;
ii) a nucleic acid according to Paragraph 46;
iii) a nucleic acid construct according to paragraph 47;
iv) a fusion protein according to any of paragraphs 34-45;
v) a biopolymer particle or particles;
vi) a coated biopolymer particle or particles according to paragraph 49; and/or
vii) an expression construct encoding a further entity that is capable of binding to an extracellular vesicle-specific surface antigen;
viii) a host cell according to paragraph 50.
   52. A method of isolating disease-specific exosomes from a sample obtained from a subject, wherein the method comprises the method of isolating extracellular vesicles from a sample according to any of paragraphs 1, or 3-32, and wherein the fusion protein comprises an extracellular vesicle binding domain that can bind to a disease-specific antigen located on the disease-specific extracellular vesicles.
   53. A method of diagnosing a disease in a subject or providing an indication that the subject likely has the disease, where the disease results in the production of disease-specific extracellular vesicles, wherein the method comprises isolating the disease-specific exosomes according to paragraph 52 and wherein where disease-specific extracellular vesicles are isolated, the subject is diagnosed with the disease or is determined to likely have the disease.
   54. The method according to paragraph 53 wherein the number of, or relative number of, disease-specific extracellular vesicles isolated is quantified.
   55. A nucleic acid construct comprising:
i) A biopolymer particle production nucleic acid construct as defined in any of the preceding paragraphs, and/or
ii) A fusion protein production nucleic acid construct as defined in any of the preceding paragraphs.

1. A method of producing biopolymer particles coated with a fusion protein, wherein the method comprises the steps of:
(i) providing a host cell that produces
a) biopolymer particles; and
b) a fusion protein capable of coating the biopolymer particles in the cells, wherein the fusion protein comprises a biopolymer particle binding domain and an extracellular vesicle binding domain and a sequence capable of being cleaved by a protease, optionally a site specific protease, optionally a TEV protease;
(ii) cultivating the host cell under conditions suitable for the production of biopolymer particles coated with the fusion protein.

2. The method of claim 1 wherein the method further comprises isolating the coated biopolymer particles from the host cell.

3. The method of any one of claims 1 or 2 wherein the biopolymer particle comprises one or more of a polyhydroxyalkanoate (PHA), a poly(L-lactide) (PLLA), a polyethylene (PE), a polystyrene (PS), or a polythioester (PTE).

4. The method of any one of claims 1-3 wherein the PHA comprises poly(3-hydroxybutyrate) (P(3HB)), poly(4-hydroxybutyrate) (P(4HB)), polyhydroxyvalerate (PHV), poly(3-hydroxyhexanoate) (P(3HHx)), poly(3-hydroxyheptanoate) (P(3HH)), poly(3-hydroxyoctanoate) (P(3HO)), poly(3-hydroxynonanoate) (P(3HN)), poly(3-hydroxydecanoate) (P(3HD)), poly(3-hydroxybutyrate-co-3-hydroxyvalerate) (PHBV), 3-hydroxybutyrate and 4-hydroxybutyrate (P3HB4HB), poly(3HB-co-3-hydroxyvalerate) (P(3HB-co-3HV)), poly(3HB-co-3-hydroxyhexanoate) (P(3HB-co-3HHx)), poly(3HB-co-3-hydroxy-4-methylvalerate) (P(3HB-co-3H4MV)), or poly(3HB-co-medium-chain-length-3HA) (P(3HB-co-mcl-3HA)).

5. The method of any one of claims 1-4 wherein the biopolymer particle is a PHA-blended biopolymer particle comprising PHA and a further biopolymer, optionally where the further biopolymer is selected from the group consisting of a poly(lactic acid)-poly(hydroxybutyrate) (PLA-PHB), starch-PHA, poly(L-lactide) (PLLA), a polyethylene (PE), a polystyrene (PS), or a polythioester (PTE).

6. The method of claim 5 wherein the PHA-blended biopolymer comprises poly(lactic acid)-poly(hydroxybutyrate) (PLA-PHB) or starch-PHA.

7. The method of any of claims 1-6 wherein the host cell comprises
i) A biopolymer particle production nucleic acid construct,
optionally wherein the biopolymer particle production nucleic acid construct expresses one or more proteins that produce one or more of a polyhydroxyalkanoate (PHA), a poly(L-lactide) (PLLA), a polyethylene (PE), a polystyrene (PS), or a polythioester (PTE),
optionally wherein the biopolymer particle production nucleic acid construct is a PHA production nucleic acid construct that expresses a phaCAB operon, optionally comprising a constitutive promoter, a synthetic ribosome binding site linked to a polynucleotide encoding a phaC gene, and/or natural ribosome binding sites linked to a polynucleotide encoding a phaA gene and a polynucleotide encoding a phaB gene;
and
ii) A fusion protein production nucleic acid construct.

8. The method of any of claims 1-7, wherein the host cell is: a bacterial cell, optionally a cyanobacterial cell; an archaeal cell, optionally a haloarchaeal cell; a fungal cell, optionally a yeast cell: or a plant cell.

9. The method of claim 8, wherein the bacterial cell is selected from the genera Alcaligenes, Azotobacter, Bacillus, Chlorogloea, Cupriavidus, Escherichia, Gloeothece, Haloferax, Halomonas, Lactobacillus, Pseudomonas, Ralstonia, Spirulina, Synechococcus, or Thermus.

10. The method of claim 8 or 9, wherein the bacterial cell is a cell selected from the group comprising Alcaligenes latus, Azotobacter chroococcum, Azotobacter vinelandii, Bacillus amyloliquefaciens DSM7, Bacillus laterosporus, Bacillus licheniformis, Bacillus macerans, Bacillus cereus, Bacillus circulans, Bacillus firmus G2, Bacillus subtilis I68, Bacillus subtilis K8, Bacillus sphaericus X3, Bacillus megaterium Y6, Bacillus coagulans, Bacillus brevis, Bacillus sphaericus ATCC 14577, Bacillus thuringiensis, Bacillus mycoides RLJ B-017, Bacillus sp. JMa5, Bacillus sp. INT005, Chlorogloea fritschii, Cupriavidus necator, Escherichia coli, Haloferax mediterraneis, Halomonas elongate, Halomonas species TD01, Halomonas sp. KM-1, Halomonas smyrnensis, Halomonas profundus, Pseudomonas aeruginosa, Pseudomonas mendocina PSU, Pseudomonas oleovorans, Pseudomonas putida, Ralstonia eutropha, or Thermus thermophilus.

11. The method of claim 8, wherein the yeast cell is a Saccharomyces cerevisiae or Pichia pastoris cell, the fungal cell is a Fusarium solani Thom cell, or the plant cell is an Arabidopsis thaliana, Camelina sativa, Nicotiana tabacum or Saccharum officinarum cell.

12. The method of any one of claims 1-11 wherein the biopolymer particles are isolated from the host cell by disrupting the cell and isolating the particles.

13. The method of claim 12 wherein disrupting the cell is performed by physical disruption.

14. The method of claim 13 wherein the physical disruption is performed by sonication, a cell press, detergent lysis, freeze-thawing, bead-beating, hypotonic cell disruption, or enzymatic disruption.

15. The method of any one of claims 12-14 wherein the isolating the particles is performed using a cell sorter, centrifugation, gravity sedimentation, electrophoresis, filtration, size exclusion chromatography or affinity chromatography.

16. The method of any one of claims 12-15 wherein the isolating the particles is performed using filtration.

17. The method of any one of claims 1-16 wherein the mean diameter of the uncoated biopolymer particle is:
a) between 50 nm and 1,500 nm, for example between 60 nm and 1,250 nm, 80 nm and 1,000 nm, 100 nm and 800 nm, 150 nm and 600 nm, 200 nm and 500 nm, 300 and 400 nm;
b) less than 1,500 nm, 1,250 nm, 1,000 nm, 800 nm, 600 nm, 500 nm, 400 nm, 300 nm, 200 nm, 150 nm, 100 nm, 80 nm, 60 nm, or less than 50 nm; and/or
c) greater than 1,500 nm, 1,250 nm, 1,000 nm, 800 nm, 600 nm, 500 nm, 400 nm, 300 nm, 200 nm, 150 nm, 100 nm, 80 nm, 60 nm, or greater than 50 nm.

18. The method of any one of claims 1-17 wherein the mean diameter of the isolating uncoated biopolymer particle is between 500 nm and 1,500 nm.

19. The method of claim 17 or 18 wherein biopolymer particle size is determined using dynamic light scattering or flow cytometry.

20. The method of any one of the claims 1-19 wherein:
a) between about 5% and 60% of the surface of the biopolymer particle is coated with the fusion protein, for example between 10% and 50%, 20% and 40%, for example 20% or 30% of the surface is coated with the fusion protein;
b) at least 5%, 10%, 20%, 30%, 40%, 50% or at least 60% of the surface is coated with the fusion protein; and/or
c) less than 60%, 50%, 40%, 30%, 20%, 10% or less than 5% of the surface is coated with the fusion protein.

21. The method of any one of claims 1-20 wherein about 20% of the surface of the biopolymer particle is coated with the fusion protein.

22. The method of claim 20 or 21 wherein the percentage of the biopolymer particles that is coated with the fusion protein is determined by transmission electron microscopy or proteolytic cleavage of the fusion protein followed by protein quantification.

23. The method of any one of claims 1-22 wherein the host cell comprises:
a) between about 5 and about 60 coated biopolymer particles, for example between about 10 and about 50, about 20 and about 40, about 30;
b) at least about 5 coated biopolymer particles, at least about 10, 20, 30, 40, 50 or at least about 60; and/or
c) less than about 60 coated biopolymer particles, less than about 50, 40, 30, 20, or less than about 5.

24. The method of any of claims 1-23 wherein the host cell comprises at least 5 biopolymer particles, preferably wherein the host cell contains 32 biopolymer particles.

25. The method of any one of claims 1-24 wherein:
i) the biopolymer binding domain comprises a domain capable of binding to one or more of a polyhydroxyalkanoate (PHA), a poly(L-lactide) (PLLA), a polyethylene (PE), a polystyrene (PS), or a polythioester (PTE); optionally comprises
a) PhaR-derived binding domain (PBD), optionally comprises or consists of SEQ ID NO: 2 or SEQ ID NO 1 or a sequence that has at least 70%, 75%, 80%, 85%, 90%, 91%, 92%, 93%, 94%, 95%, 96%, 97%, 98%, 99%, or 100% sequence identity to SEQ ID NO: 1 or SEQ ID NO: 2;
b) a phasin, optionally a PhaR, a PhaP, a PhaQ, a PhaF, a PhaI, or an inactive PhaZ1;
c) IbpA (HspA); or
d) PhaC,
and/or
ii) the extracellular vesicle binding domain is selected from a protein, a protein fragment, a binding domain, a target-binding domain, a binding protein, a binding protein fragment, an affibody, an antibody, an antibody fragment, an antibody heavy chain, an antibody light chain, a single chain antibody, a single-domain antibody, a Fab antibody fragment, an Fc antibody fragment, an Fv antibody fragment, a F(ab′)2 antibody fragment, a Fab′ antibody fragment, a single-chain Fv (scFv) antibody fragment, a camelid antibody, an IgNAR Shark antibody, a DARPin, a nanobody, an antibody binding domain, an antigen, an antigenic determinant, an epitope, a hapten, an immunogen, an immunogen fragment, biotin, a biotin derivative, an avidin, a streptavidin, a substrate, an enzyme, an abzyme, a co-factor, a receptor, a receptor fragment, a receptor subunit, a receptor subunit fragment, a ligand, an inhibitor, a hormone, a lectin, a polyhistidine, a coupling domain, a DNA binding domain, a FLAG epitope, a cysteine residue, a library peptide, a reporter peptide, and an affinity purification peptide, or a combination thereof;
and
optionally wherein the fusion protein further comprises:
iii) a functionalisation domain, optionally wherein the functionalisation domain is a membrane disrupting peptide or a cell targeting peptide,

26. The method of any of claims 1-25 wherein the extracellular vesicle binding domain is capable of binding specifically to an extracellular vesicle-specific surface antigen.

27. The method of any of claims 1-26 wherein the biopolymer particle binding domain is located at the N-terminus of the fusion protein and the extracellular vesicle binding domain is located at the C-terminus of the fusion protein.

28. The method of any of claims 1-26 wherein the extracellular vesicle binding domain is located at the N-terminus of the fusion protein and the biopolymer particle binding domain is located at the C-terminus of the fusion protein.

29. The method of any one of claims 1-28 wherein the biopolymer particle binding domain is fused to the extracellular vesicle binding domain via a linker peptide, optionally where the fusion protein comprises a functionalisation domain the biopolymer particle binding domain is fused to the functionalisation domain via a linker peptide.

30. The method of claim 29 wherein the linker peptide is more than 12 amino acids in length, for example more than 15, 20, 25, 30, 35, 40, 45, 50 60, 70, 80, 90, 100, 110, 120, 130, 140, 150 amino acids in length, and/or between around 12-112 amino acid residues in length, and optionally comprises small, non-polar and/or small, polar amino acids.

31. The method of any of claims 1-30 wherein the fusion protein comprises a protease site, optionally a TEV protease site.

32. The method of any one of claims 1-31 wherein the sequence capable of being cleaved by a protease, optionally a site specific protease, optionally a TEV protease is located in the linker peptide.

33. The method of any one of claims 1-32 wherein the biopolymer particle binding domain comprises a PHA binding domain of a phasin repressor protein, PhaR.

34. The method of claim 33 wherein the PHA binding domain of a phasin repressor protein, PhaR, lacks DNA-binding activity.

35. The method of any one of claims 1-34 wherein the extracellular vesicle binding domain is selected from a protein, a protein fragment, a binding domain, a target-binding domain, a binding protein, a binding protein fragment, an affibody, an antibody, an antibody fragment, an antibody heavy chain, an antibody light chain, a single chain antibody, a single-domain antibody, a Fab antibody fragment, an Fc antibody fragment, an Fv antibody fragment, a F(ab′)2 antibody fragment, a Fab′ antibody fragment, a single-chain Fv (scFv) antibody fragment, a camelid antibody, an IgNAR Shark antibody, a DARPin, a nanobody, an antibody binding domain, an antigen, an antigenic determinant, an epitope, a hapten, an immunogen, an immunogen fragment, biotin, a biotin derivative, an avidin, a streptavidin, a substrate, an enzyme, an abzyme, a co-factor, a receptor, a receptor fragment, a receptor subunit, a receptor subunit fragment, a ligand, an inhibitor, a hormone, a lectin, a polyhistidine, a coupling domain, a DNA binding domain, a FLAG epitope, a cysteine residue, a library peptide, a reporter peptide, and an affinity purification peptide, or a combination thereof.

36. The method of any one of claims 1-35 wherein the extracellular vesicle binding domain is an affibody.

37. The method of any one of claims 1-36 wherein the method produces at least two different fusion protein coated biopolymer particles.

38. The method according to claim 38 wherein the at least two different fusion protein coated biopolymer particles comprise different biopolymer particles.

39. The method according to claim 37 or 38 wherein the at least two different fusion protein coated biopolymer particles comprise different fusion proteins.

40. A fusion protein as defined in any of claims 1-39.

41. A nucleic acid encoding the fusion protein of claim 40.

42. A nucleic acid construct comprising:
i) a nucleic acid sequence encoding a fusion protein as defined in claim 40;
ii) a nucleic acid sequence encoding a further entity that is capable of binding to an extracellular vesicle-specific surface antigen; and
optionally a nucleic acid encoding a biopolymer synthase
operably linked to at least one promoter.

43. The nucleic acid of claim 41 or 42 wherein the promoter is an inducible promoter, a synthetic promoter, a viral promoter or a phage promoter.

44. A nucleic acid construct comprising:
i) A biopolymer particle production nucleic acid construct as defined in any of the preceding claims, and/or
ii) A fusion protein production nucleic acid construct as defined in any of the preceding claims.

45. A biopolymer particle coated with:
i) one or more fusion proteins according to claim 40; or
ii) one or more fusion proteins as defined in any of claim 40 and further comprising the further entity that is capable of binding specifically to an extracellular vesicle-specific surface antigen.

46. The coated biopolymer particle according to claim 45 wherein the biopolymer particle was produced according to the method of any of claims 1-39.

47. The coated biopolymer particle according to claim 45 wherein the biopolymer particle was produced by a cell-free method comprising the steps of:
(i) providing a solution comprising
a) at least one biopolymer particle production nucleic acid construct, that comprises a first promoter, optionally a first inducible or repressible promoter suitable for the production of biopolymer particles; and
b) at least one fusion protein production nucleic acid construct that comprises a second promoter, optionally a second inducible or repressible promoter
(ii) maintaining the solution under conditions suitable for expression of the at least one biopolymer particle production nucleic acid construct and the at least one fusion protein production nucleic acid construct, and for formation of biopolymer particles coated with the fusion protein; and optionally
(iii) isolating the coated biopolymer particles from the solution.

48. The coated biopolymer particle according to claim 45 wherein the biopolymer particle was produced by a method comprising the steps of:
(i) providing a biopolymer particle;
(ii) providing a fusion protein capable of coating the biopolymer particle; and
(iii) contacting the biopolymer particle with the fusion protein under conditions suitable for formation of biopolymer particles coated with the fusion protein.

49. A host cell as defined in any of claims 1-39.

50. A host cell that comprises:
i) A biopolymer particle production nucleic acid construct, and
ii) A fusion protein production nucleic acid construct.

51. A host cell comprising a nucleic acid according to any of claims 41-44, optionally operably linked to an inducible promoter,
wherein the host cell optionally comprises one or more nucleic acids that drive production of the biopolymer particle, optionally under the control of an inducible promoter,
optionally wherein the inducible promoter that is operably linked to the nucleic acid according to any of claims 41-44 and the inducible promoter that drives production of the biopolymer particle are induced by different inducers.

52. A kit comprising any one or more of:
i) an expression construct comprising a biopolymer production module, optionally wherein the biopolymer production module produces one or more of a polyhydroxyalkanoate (PHA), a poly(L-lactide) (PLLA), a polyethylene (PE), a polystyrene (PS), or a polythioester (PTE),
optionally wherein the biopolymer production module is a PHA production module that expresses a phaCAB operon, optionally comprising
a constitutive promoter,
a synthetic ribosome binding site linked to a polynucleotide encoding a phaC gene, and/or
natural ribosome binding sites linked to a polynucleotide encoding a phaA gene and a polynucleotide encoding a phaB gene;
ii) a nucleic acid according to any of claims 41-44;
iii) a fusion protein according to claim 40;
iv) a biopolymer particle or particles;
v) a coated biopolymer particle or particles according to any of claims 45-48; and/or
vii) an expression construct encoding a further entity that is capable of binding to an extracellular vesicle-specific surface antigen;
viii) a host cell according to any of claims 49 or 51.

53. A method for isolating extracellular vesicles from a sample, the method comprising:
(i) providing a composition of coated biopolymer particles, the biopolymer particles being coated with a fusion protein;
(ii) contacting the composition comprising the coated biopolymer particles with the sample comprising extracellular vesicles under conditions which allow the formation of a coated biopolymer particle-extracellular vesicle complex; and
(iii) isolating the coated biopolymer particle-extracellular vesicle complex.

54. A method for functionalising the surface of extracellular vesicles, the method comprising:
(i) providing a composition of coated biopolymer particles, the biopolymer particles being coated with a fusion protein that comprises a functionalisation domain;
(ii) contacting the composition comprising the coated biopolymer particles with a sample comprising extracellular vesicles under conditions which allow the formation of a coated biopolymer particle-extracellular vesicle complex; and
(iii) isolating the coated biopolymer particle-extracellular vesicle complex.

55. The method according to any of claims 53 or 54 wherein the method comprises releasing the extracellular vesicles from the coated biopolymer particle-extracellular vesicle complex, optionally wherein the releasing does not involve the use of chelating agents.

56. The method according to claim 55 wherein the fusion protein comprises a sequence capable of being cleaved by a protease, optionally a site specific protease, optionally a TEV protease, and said releasing involves cleavage of said sequence.

57. The method according to claim 53-56 further comprising:
(iv) processing the coated biopolymer particle-extracellular vesicle complex so as to provide a) a functionalisation domain-associated extracellular vesicle and b) a fusion protein portion-associated biopolymer particle,
optionally wherein the fusion protein comprises a sequence capable of being cleaved by a protease, optionally a site specific protease, optionally a TEV protease, said processing involves cleavage of said sequence.

58. The method according to any of claims 53-57 wherein the coated biopolymer particles have been made according to the method of any of claims 1-39.

59. The method of any of claims 53-58 wherein the extracellular vesicle is a microvesicle, an apoptotic body, an ectosome, an exosome, an exomere, a small oncosomes, a large oncosome or an exosome mimetic.

60. The method of any one of claims 53-59 wherein the extracellular vesicle is an exosome.

61. The method of any one of claims 53-60 wherein the contacting the composition comprising the coated biopolymer particles with a sample comprising extracellular vesicles, occurs in aqueous solution, at a temperature between 4° C.-60° C., at a pH between 6.0-8.5.

62. The method of any one of claims 53-57 or 59-61 wherein the biopolymer particles have been formed by a cell-free method comprising the steps of:
(i) providing a solution comprising
a) at least one biopolymer particle production nucleic acid construct, that comprises a first promoter, optionally a first inducible or repressible promoter suitable for the production of biopolymer particles; and
b) at least one fusion protein production nucleic acid construct that comprises a second promoter, optionally a second inducible or repressible promoter
(ii) maintaining the solution under conditions suitable for expression of the at least one biopolymer particle production nucleic acid construct and the at least one fusion protein production nucleic acid construct, and for formation of biopolymer particles coated with the fusion protein; and optionally
(iii) isolating the coated biopolymer particles from the solution.

63. The method of any one of claims 53-57 or 59-61 wherein the coated biopolymer particles have been formed by a method comprising the steps of:
(i) providing a biopolymer particle;
(ii) providing a fusion protein capable of coating the biopolymer particle; and
(iii) contacting the biopolymer particle with the fusion protein under conditions suitable for formation of biopolymer particles coated with the fusion protein.

64. A method of isolating disease-specific extracellular vesicles from a sample obtained from a subject, wherein the method comprises the method of isolating extracellular vesicles from a sample according to any of claims 53-63 and wherein the fusion protein comprises an extracellular vesicle binding domain that can bind to a disease-specific antigen located on the disease-specific extracellular vesicles.

65. A method of diagnosing a disease in a subject or providing an indication that the subject likely has the disease, where the disease results in the production of disease-specific extracellular vesicles, wherein the method comprises isolating the disease-specific extracellular vesicles according to claim 64 and wherein where disease-specific extracellular vesicles are isolated, the subject is diagnosed with the disease or is determined to likely have the disease.

66. The method according to claim 65 wherein the number of, or relative number of, disease-specific extracellular vesicles isolated is quantified.

67. A method of diagnosing a disease in a subject or providing an indication that the subject likely has the disease, where the disease results in the an increase or decrease in the number of extracellular vesicles, optionally disease specific extracellular vesicles, wherein the method comprises isolating extracellular vesicles according to any of claims 53-63, optionally disease-specific extracellular vesicles according to claim 64 and quantifying the isolated extracellular vesicles.


SEQUENCE LISTING (2 sequences)

SEQ ID NO: 1 - protein, 183 aa, Cupriavidus
  Polyhydroxyalkanoate synthesis repressor PhaR
MATTKKGAERLIKKYPNRRLYDTQTSTYITLADVKQLVMDSEEFKVVDAKSGDELTRSILLQIILEEETGGVPMFSSAMLSQIIRFYGHAMQGMMGTYLEKNIQAFIDIQNKLAENSKGLYSGETFSPDMWSQFMNMQGPMMQGMMSNYIEQSKNLFVQMQEQMQSQAKNMFGTFPFNQPDKK

SEQ ID NO: 2 - protein, 92 aa, Artificial Sequence
  PhaR-derived binding domain
MFSSAMLSQIIRFYGHAMQGMMGTYLEKNIQAFIDIQNKLAENSKGLYSGETFSPDMWSQFMNMQGPMMQGMMSNYIEQSKNLFVQMQEQMQ